(19) United States
(12) Patent Application Publication
(10) Pub. No.: US 2025/0207133 A1
(43) Pub. Date: Jun. 26, 2025

(54) CONSTRUCTS, VIRAL VECTORS AND PHARMACEUTICAL COMPOSITIONS COMPRISING LONG NONCODING RNA ANRIL FOR TREATMENT OF DISEASE

(71) Applicant: Trustees of Tufts College, Medford, MA (US)

(72) Inventors: Jinkun Chen, Medford, MA (US); Qisheng Tu, Medford, MA (US); Xiaofang Zhu, Medford, MA (US); Yao Liu, Medford, MA (US)

(21) Appl. No.: 18/999,652

(22) Filed: Dec. 23, 2024

Related U.S. Application Data
(60) Provisional application No. 63/613,501, filed on Dec. 21, 2023.

(51) Int. Cl. C12N 15/113; A61K 38/17; A61K 48/00; A61P 1/02; A61P 19/08; A61P 29/00; C12N 15/86; C12Q 1/6883; G01N 33/68

(52) U.S. Cl. CPC C12N 15/113; A61K 38/1709; A61K 48/005; A61K 48/0075; A61P 1/02; A61P 19/08; A61P 29/00; C12N 15/86; C12Q 1/6883; G01N 33/6893; C12N 2310/113; C12N 2750/14143; G01N 2800/042; G01N 2800/10; G01N 2800/18; G01N 2800/323

(57) ABSTRACT

Disclosed herein are systems and platforms for a construct including a heterologous promoter operably connected to a nucleic acid sequence encoding an ANRIL RNA or portion thereof or a Trefoil Factor Family 2 protein (Tff2).

### CROSS REFERENCE TO RELATED APPLICATIONS

This application claims priority benefit from U.S. Application Ser. No. 63/613,501, filed Dec. 21, 2023. The entirety of which is incorporated herein by reference.

### STATEMENT REGARDING FEDERALLY SPONSORED RESEARCH

This invention was made with government support under DE030074 awarded by the National Institutes of Health. The government has certain rights in the invention.

### REFERENCE TO A SEQUENCE LISTING

A sequence listing (file name: 166118_01502.xml; size: 146,545 bytes; date generated: Dec. 23, 2024) is hereby incorporated by reference in its entirety.

### BACKGROUND

Noncoding RNAs (ncRNAs) are a large segment (more than 80%) of the transcriptome that lack apparent protein encoding capability but are functionally important. Long noncoding RNAs (lncRNAs) are a subgroup of ncRNAs with a length of more than 200 nucleotides. Emerging evidence suggests that lncRNAs participate in a wide repertoire of genome organization and life processes such as growth and development, cell proliferation, differentiation, immune response, and certain diseases, such as cancer and cardiovascular diseases.

LncRNA ANRIL (referred to herein as ANRIL), situated on chromosome 9p21.3 in humans, is a lncRNA encoded in the opposite direction within the INK4/ARF locus. ANRIL is the antisense lncRNA of cyclin-dependent kinase inhibitor 2A (CDKN2A) and CDKN2B, both of which are protein coding genes situated in the INK4 locus. ANRIL has been proven to be a shared risk factor for atherosclerosis, periodontitis, diabetes, and cancer.

The inventors demonstrate that deficiency on the long non-coding RNA ANRIL (ADPC in the mouse) exacerbates periodontitis by increasing alveolar bone loss and inflammatory infiltration of the periodontal tissue. They further demonstrate that this damage can be reversed by administration of a composition comprising ANRIL and that the long non-coding RNA can be delivered using a viral vector.

### SUMMARY

Provided here are constructs, viral vectors and pharmaceutical compositions comprising a heterologous promoter operably connected to a nucleic acid sequence encoding an ANRIL RNA or a Tff2 protein. The sequence encoding the ANRIL RNA includes at least one of SEQ ID NO: 1-32 or a sequence having at least 90% identity to at least one of SEQ ID NO: 1-32 or portions thereof. The nucleic acid sequence encoding the Tff2 protein includes at least one nucleic acid sequence selected from SEQ ID NO: 89-90 or a sequence having at least 90% identity to at least one of SEQ ID NO: 89-90 or a nucleic acid sequence encoding the polypeptide of SEQ ID NO: 91-92 or a sequence having at least 90% identity to one of SEQ ID NO: 91-92. The viral vector may be an Adeno-associated viral vector.

Also provided are methods of using any one of the constructs, the viral vectors or the pharmaceutical compositions to treat a condition. The methods include administering a therapeutically effective amount of the construct, the viral vector or the composition to a subject in need of treatment for the condition. The conditions include periodontitis, bone or metabolic disorders and atherosclerosis. The methods may further include measuring the level of the long noncoding RNA ANRIL or Tff2 protein or mRNA in a subject to determine or diagnose the presence of the condition or disease in the subject. A low level of ANRIL RNA or Tff2 protein or mRNA is indicative of the condition or disease.

### BRIEF DESCRIPTION OF THE DRAWINGS

FIGS. 1A-1D show mRNA expression of Runx2 and ANRIL (FIG. 1A), Runx2 and APDC (FIG. 1B), C/EBPα and APDC (FIG. 1C) and cathepsin K and APDC (FIG. 1D) analyzed via RT-RCR. The results were normalized using negative control group. All data were statistically analyzed via GraphPad Prism 9.1.2).

FIGS. 2A-2C show the mRNA expression of Alp (FIG. 2A), Ucp1 (FIG. 2B), Mmp9 (FIG. 2C) and APDC in primary cells from C57BL/6J were analyzed via RT-RCR.

FIG. 3A-3D. (FIG. 3A) PCR of mouse tail DNA showing the genotyping of the APDC knockout: homozygotes (−/−), heterozygotes (+/−) and wild type (+/+). FIG. 3B. PCR verifying the IncR-APDC overexpression plasmid was constructed successfully. FIG. 3C. The Differentiation mRNA expression, ARS, oil red O staining and trap staining results were presented to describe the effects in osteogenesis, adipogenesis and osteoclastogenesis which was related to deletion of IncR-APDC. FIG. 3D. The Differentiation mRNA expression, ARS, oil red O staining and trap staining results were presented to describe the effects in osteogenesis, adipogenesis and osteoclastogenesis which was related to overexpression of IncR-APDC. (All results were normalized using control group and statistically analyzed via GraphPad Prism 9.1.2.).

FIG. 4. The proliferation of BMSCs gathered from WT and APDC-KO mice were analyzed via OD of CCK-8.

FIGS. 5A-5E. (FIG. 5A). The histological HE-stained and OCN IHC staining femurs of 8-week-group were presented under different magnifications. (FIG. 5B). The mRNA expression of bone tissue from WT/APDC-KO mice were analyzed via RT-RCR. (FIG. 5C) Expression of TLR4 and MyD88 in BMMs from WT/APDC-KO mice. (FIG. 5D) Expression of OPG, TLR4 and MyD88 in osteogenesis-induced BMSCs from WT/APDC-KO mice. (FIG. 5E) Expression of p38, p-p38 and TLR4 in osteoclastogenesis-induced BMMs from WT/APDC-KO mice. (All results were normalized using WT group and statistically analyzed via GraphPad Prism 9.1.2.).

FIG. 6. The histological HE-stained and OCN IHC staining femurs of 4-week-group were presented under different magnifications.

FIGS. 7A-7C. (FIG. 7A) The body type and weight of male mice was shown. (FIG. 7B) The histological HE-stained wat from Inguinal region was presented under different magnifications. (FIG. 7C) The mRNA expression of WAT from WT/APDC-KO mice were analyzed via RT-RCR. (All results were normalized using WT group and statistically analyzed via GraphPad Prism 9.1.2.).

FIGS. 8A-8G. (FIG. 8A) KDM6B expression in WT/APDC-KO mice analyzed via RT-RCR. (FIG. 8B) Analyzed KDM6B expression in BMSCs with overexpression of IncR-APDC via RT-RCR. (FIG. 8C) The miR99a expression in WT/APDC-KO mice. (FIG. 8D) Analyzed miR99a expression in BMSCs with overexpression of IncR-APDC via RT-RCR. (FIG. 8E) The prediction of binding site between lncR-APDC and miR-99a using MicroInspector online software (bioinfo.uni-plovdiv.bg/microinspector) (SEQ ID NO: 93 and 94). (FIG. 8F) Wild-type and mutant lncR-APDC inserted into a Luciferase Vector, were transfected into BMSCs, which were cotransfected with either control or miR-99a mimic. miR-99a effectively suppressed luciferase reporter activity. (FIG. 8G) IncR-APDC could regulate osteogenesis by targeting miR99a, which can regulate osteogenic differentiation through KDM6. (All results were normalized using negative control group and statistically analyzed via GraphPad Prism 9.1.2.).

FIG. 9. The histological HE-stained and OCN IHC staining alveolar bone of 4-week-group (top) and 8-week-group (bottom) were presented under different magnifications.

FIG. 10A-10E. EP model on APDC deficiency mice and control mice. (FIG. 10A) The digital (.stl) file and 3D printed copy of the surgical tools. (FIG. 10B) Morphology analysis showed the distance from the alveolar bone crest (ABC) to the cementoenamel junction (CEJ) on the buccal sides and palatal sides (n=7). (FIG. 10C) the expression level of APDC in wildtype and APDC KO mouse. (FIG. 10D) HE staining of the periodontal tissues. The zoom-ins show the inflamed periodontal tissues. Green triangles show cementum resorption (discontinuous cementum). (FIG. 10E) TRAP staining shows the TRAP positive cells (red) on the alveolar bone surface in APDC KO and control group at 3- and 6-weeks. Original magnification 400×. Scale bars=200 m. Values are shown as mean±SD. *p<0.05, **p<0.01.

FIGS. 11A-11F. The impact of APDC on inflammation related cytokines and bone metabolism. (FIG. 11A) the heatmap of untreated (day 0) BMSCs (upper) and osteogenic differentiated (day 3) BMSCs (lower) comparing between APDC KO and control mice. (FIG. 11B) the volcano plot with top genes marked (WT vs. KO). (FIG. 11C) the GO enrichment results at untreated (upper) and osteogenesis-induced (lower) groups. (FIG. 11D) the mRNA expression level of the osteogenic markers. (FIG. 11E) ALP and ARS staining results (upper) and the mRNA expression level of osteogenesis related genes (lower) for APDC KO and control mice. (FIG. 11F) the serum level of cytokines (IL-10, IL-6, CXCL1, TNF-α, IL-10, IL-2, IL-5 and IFN-γ) (n=5). Values are shown as mean±SD. *p<0.05, ***p<0.001, ****p<0.0001.

FIGS. 12A-12E. Overview of the 25,161 single cells from periodontal tissue of APDC-KO mice and wildtype mice (n=4 per group). (FIG. 12A) Uniform Manifold Approximation and Projection (UMAP) representation of the 25,161 cells, colored by cell type annotation. (FIG. 12B) stacked violin plots showing the expression scores of selected marker gene sets across all 10 clusters. (FIG. 12C) the bar plots indicating the percentage of each cell type. (FIG. 12D) UMAP plot representation the distribution of 11,917 cells of WT group (left) and 13,244 cells of APDC-KO group (right) from the gingival tissue in the 10 clusters. (FIG. 12E) the correlation matrix of the 10 cell types.

FIGS. 13A-13L. The proportion and function changes of the immune cells in the periodontitis gingival tissue due to the APDC deficiency. (FIGS. 13A-13D) T cell: (FIG. 13A) the subclusters of T cells. (FIG. 13B) the violin plots showed the expression of the marker genes. (FIG. 13C) the WT and KO group of T cells. (FIG. 13D) the DEGs of CD4+ and CD8+ cells. B cell. (FIGS. 13E-13F) B cell: (FIG. 13E) the UMAP (left) and pie chart (right) showed the proportion of WT and KO B cells. (FIG. 13F) the violin plots showed the gene expression level of KO and WT at different stages of B cells. (FIG. 13F) Macrophage: (FIG. 13G) the subclusters of macrophages. (FIG. 13H) the WT and KO group of macrophages. (FIG. 13I) the highly expressed genes of each subcluster. (FIG. 13J) the M1 and M2 macrophages marker genes expression in the subclusters. (FIGS. 13K-13L) Neutrophil: (FIG. 13K) the UMAP of all neutrophils and divided by WT and KO. (FIG. 13L) dot plot showed highly expressed genes of cluster 1 and 0.

FIGS. 14A-14G. Endothelial cells, fibroblasts, myofibroblasts and pericytes are in the inflamed issues of both groups. (FIG. 14A) Endothelial cells are the biggest cellular component in the inflamed gingival samples. Total of 5 subclusters were identified. (FIG. 14B) Subcluster_1, which highly expressed genes, Selp, Ackr1, and Sele, are reported to be highly associated with immune regulation during PD development and indicates the tissue samples are inflamed. (FIG. 14C, FIG. 14D) We further checked the related gene expression and cell counts in both KO and WT group. There is no significant disparity between two groups for fibroblasts (FIG. 14E), myofibroblasts (FIG. 14F), and pericytes (FIG. 14G) Fibroblasts are centrally involved in the wound healing response and remodeling of the periodontal tissue. During the remodeling phase of wound healing, a specific subtype of fibroblast may emerge, which is the Myofibroblast. Myofibroblasts may also derive from alternative sources, including mesenchymal stem cells, pericytes, and epithelial cells. It was recently reported that pericytes possess multilineage differentiation capacity and can be the source of tissue stem cells and/or progenitor cells, making them similar to periodontal ligament stem cells (PDLSCs). In our data, the gene profiling and cell counts for these three cell populations are consistent with other periodontitis related studies and did not show a disparity between APDC KO and wildtype group.

FIG. 15. The DEGs analysis of the B cells, endothelial cells, epithelial cells, macrophages, NK cells, Neutrophils and T cells.

FIGS. 16A-16G. Sub-clustering of the epithelial cells. (FIG. 16A) the subclusters of epithelial cells. (FIG. 16B) epithelial cells divided by WT and KO (right). (FIG. 16C) the classic distinct markers of epithelium. (FIG. 16D) Epi_1 cluster highly expressed Krt 8 and Krt 18. (FIG. 16E) Epi_2 cluster highly expressed Krt 14 and Krt5. (FIG. 16F) the DEGs of Epi_1 and Epi_2. (FIG. 16G) the GO enrichment results of Epi_1 and Epi_2.

FIG. 17. The DEGs analysis of the fibroblasts, myofibroblasts and pericytes.

FIGS. 18A-18F. The mRNA and protein expression of Tff2 and its predicted binding site with APDC. (FIG. 18A) the expression of Tff2 in WT and KO group across various cell types. (FIG. 18B) the volcano plot illustrated the expression level of DEGs. (FIG. 18C) the predicted binding site of Tff2 (top) and APDC (bottom) (SEQ ID NOS: 97 and 98). (FIG. 18D) the heatmap showed the lowest binding energy and highest portability at the location of 1985 to 2026 on APDC. (FIG. 18E) the Tff2 expression level in EP maxilla and gingival. (FIG. 18F) the immunohistochemistry staining showed the Tff2 protein level in WT and KO groups.

FIGS. 19A-19F. The cell-cell communications among different cell types. (FIG. 19A) the plot results of top ligand-receptor pairs between T cell and other cell types in WT and KO group. (FIG. 19B) the plot results of top ligand-receptor pairs between Neutrophil and other cell types in WT and KO group, (FIG. 19C) the plot results of top ligand-receptor pairs between macrophage and other cell types in WT and KO group. (FIG. 19D) the plot results of top ligand-receptor pairs between epithelial cells and other cell types in WT and KO group. (FIG. 19E) The expression of Ilb1 in WT and KO respectively. (FIG. 19F) The expression of Pglyrp1 in WT and KO respectively.

FIGS. 20A-20I. The AAV9-CAG-APDC attenuates periodontal bone destruction. (FIG. 20A) the AAV-APDC was administrated through microinjection on gingiva. (FIG. 20B) the timeline of the treatment. (FIG. 20C) the IVIS imaging of the RFP expression in AAV and PBS group. (FIG. 20D) the APDC expression level in wild type groups. (FIG. 20E) the APDC expression level in APDC KO groups. (FIG. 20F) Micro-CT showed the bone resorption level with and without AAV treatment in WT mice. (FIG. 20G) Micro-CT showed the bone resorption level with and without AAV treatment in KO mice (n=4-6). (FIG. 20H) The mRNA expression of Tff2 after AAV-APDC treatment in WT group. (FIG. 20I) The mRNA expression of Tff2 after AAV-APDC treatment in KO group. *p<0.05.

FIGS. 21A-21B. Primary gingival epithelial cells transfected with Tff2-encoding plasmid and analyzed with PCR (FIG. 21A) and immunofluorescence staining (FIG. 21B). (FIG. 21A) shows high expression of Tff2 in E DNA Tff2 cells. (FIG. 21B) shows immunofluorescence staining of Tff2 (red) and nuclei, stained with DAPI (blue). (400×).

FIG. 22. Wound healing scratch assay with cells transfected with an empty plasmid (E DNA NC) or with a Tff2-encoding plasmid (E DNA Tff2). E DNA Tff2 cells exhibited faster growth and migration compared to the control cells after 24 h and 48 h.

FIGS. 23A-23C. The expression of Tff2 in the E DNA NC cells and E DNA NC L100 cells (FIG. 23A), in the E DNA Tff2 cells and EDNA Tff2 L100 cells (FIG. 23B). (FIG. 23C) The IF staining of Tff2 (red) and DAPI (blue). (400×).

FIGS. 24A-24C. (FIG. 24A) Real-time PCR showed the expression of IL-6 in two groups of cells. (FIG. 24B). ELISA revealed the extracellular release of IL-6 in the two groups of cells. (FIG. 24C) Immunofluorescence staining of IL-6 (green) in the two groups of cells. The nuclei were stained with DAPI (blue). (400×).

FIGS. 25A-25B. (FIG. 25A) The low expression of Tff2 in E siRNA Tff2 cells. (FIG. 25B) Immunofluorescence staining of Tff2 (red) in the two groups of cells. The nuclei were stained with DAPI (blue). (400×).

FIG. 26. Reduced proliferation rate following Tff2 silencing.

FIGS. 27A-27H. The expression of Tff2 (FIGS. 27A and 27E), Muc6 (FIGS. 27B and 27F), Krt8 (FIGS. 27C and 27G), and MMP3 (FIGS. 27D and 27H) was detected by Real-time PCR analysis in cells with overexpressed Tff2 (FIGS. 27A-27D) or silenced Tff2 (FIGS. 27E-27H).

FIGS. 28A-28B. (FIG. 28A) The differentially expressed genes (DEGs) between E siRNA NC and E siRNA Tff2. (FIG. 28B). The DEGs were enriched in signaling pathways after silencing Tff2 in oral epithelial cells.

FIG. 29. Micro-CT reconstruction and bone loss level.

### DETAILED DESCRIPTION

The present invention provides constructs and viral vectors for expression of ANRIL RNA and Trefoil factor family 2 (Tff2) protein and compositions comprising constructs and viral vectors for generating ANRIL RNA and Tff2 protein. Methods of using these constructs, viral vectors and compositions to treat or diagnose diseases are also provided.

As described in the Examples, the present inventors have identified a long non-coding RNA ANRIL (lncRNA-ANRIL). LncRNA-ANRIL, situated on chromosome 9p21.3 in humans, is a lncRNA encoded in the opposite direction within the INK4/ARF locus. ANRIL is the antisense lncRNA of cyclin-dependent kinase inhibitor 2A (CDKN2A) and CDKN2B, both of which are protein coding genes situated in the INK4 locus. ANRIL has been shown to be a shared risk factor for atherosclerosis, periodontitis, diabetes, and cancer. ANRIL is the best replicated genetic locus of atherosclerosis-associated coronary artery disease (CAD) and PD. The core risk haplotype shared between CAD and PD is located at the 3′ end of ANRIL, which implies ANRIL is a prime functional candidate involved in the risk mediating mechanism. It can modulate genes and pathways related to inflammation, cell cycle regulation, and atherosclerosis. Additionally, studies suggest a bi-directional association between periodontitis, diabetes, and CAD.

ANRIL deficiency exacerbates periodontitis by increasing alveolar bone loss and inflammatory infiltration of the periodontal tissue. Deleting lncRNA-ANRIL reduces the population of B cells and CD8+ cytotoxic T cells while increasing the M1/M2 ratio and recruitment of neutrophils to inflamed periodontal tissues. The inventors delivered AAV9-CAG-ANRIL to the experimental periodontitis site via gingival injection. The data presented in the Examples demonstrated the potential of lncRNA-ANRIL in the treatment of periodontal disease. Additionally, lncRNA-ANRIL plays a regulatory role in bone and adipose tissue metabolism. LncRNA-ANRIL can enhance osteogenesis through miR-99a/KDM6B/How/Runx2 pathways. It can also inhibit osteoclastogenesis through MAPK/p-38 and TLR4/MyD88 signaling pathways and regulate adipogenesis via APN-related pathways. Therefore, lncRNA-ANRIL is a promising therapeutic target for bone and fat metabolic diseases as well as periodontitis and may be useful in treating a wide range of diseases.

In the Examples, a unique interaction between lncRNA-APDC and Trefoil Factor 2 (Tff2) was identified. In lncRNA-APDC mice, Tff2 expression is significantly elevated at both RNA and protein levels across various tissue types. Tff2 encodes a small, secreted protein essential for mucosal protection and repair, contributing to epithelial barrier integrity. Its anti-inflammatory properties-including immune modulation and reduction of tissue inflammation suggest a potential role in mitigating disease progression. Tff2 overexpression and silencing models were developed and tested and demonstrated Tff2's anti-inflammatory and wound healing effects on epithelial cells. Thus also provided are constructs vectors and compositions comprising a heterologous promoter operably connected to a nucleic acid sequence encoding Tff2 to allow for expression of tff2 RNA and protein in cells to treat conditions ranging from periodontitis to diabetes, inflammation and atherosclerosis.

lncRNA-ANRIL and Tff2 Protein Encoding Compositions:

In a first aspect, the present invention provides compositions and constructs comprising a nucleotide sequence encoding ANRIL RNA. Human ANRIL RNA is provided as SEQ ID NO: 1-32. ANRIL is a genetic risk factor for atherosclerosis, coronary artery disease (CAD), myocardial infarction (MI), periodontitis (PD), diabetes and cancer. Notably, the mouse lncRNA-ANRIL ortholog is AK148321, also referred to as IncR-APDC (SEQ ID NO: 33). However, in the present application, the nomenclatures ANRIL and APDC are used interchangeably to refer to the RNA and the nucleotide sequence encoding the RNA and are not necessarily used to indicate the species from which the sequences is derived.

In one embodiment, the present technology provides an isolated polynucleotide or construct comprising a polynucleotide sequence encoding an ANRIL RNA comprising at least one of SEQ ID NO: 1-32 operably linked to a heterologous promoter capable of expression of the polynucleotide in a cell. SEQ ID NOs: 1-32 represent sequence variants of ANRIL currently identified in the population. Alternatively, the present technology provides an isolated polynucleotide comprising a polynucleotide sequence encoding APDC comprising SEQ ID NO: 33 (the mouse homolog) operably linked to a heterologous promoter to allow expression of the encoded RNA in a cell. Due to the noted heterogeneity of this sequence in the population, sequences having at least 95%, 96%, 97%, 98%, 99% sequence identity to any one of SEQ ID NOs: 1-33 are also provided. Functional portions of the sequences encoding ANRIL may also be sufficient to yield a therapeutic effect in the methods provided herein. The sequences encoding ANRIL may be 50%, 55%, 60%, 65%, 70%, 75%, 80%, 85%, 90%, 95% of the full length ANRIL sequence of SEQ ID NOs:1-33.

In another aspect, the present invention provides compositions and constructs comprising a nucleotide sequence encoding TFF2 polypeptide. Polynucleotide sequences encoding human TFF2 and mouse Tff2 are provided as SEQ ID NO: 89 and 90 and the amino acid sequence are provided as SEQ ID NO: 91 and 92. In the present application, the nomenclatures of Tff2 and TFF2 are used interchangeably to refer to the DNA or RNA sequences including mRNA nucleotide sequences encoding the protein and are not necessarily used to indicate the species from which the sequence is derived.

In one embodiment, the present technology provides an isolated polynucleotide or construct comprising a polynucleotide sequence encoding a TFF2 protein. The nucleotide sequence encoding the TFF2 protein may comprise at least one of SEQ ID NOs: 89-90 or another nucleic acid sequence or polynucleotide encoding the TFF2 protein of SEQ ID NO: 91 or 92. The nucleic acid sequence is operably linked to a heterologous promoter capable of expressing an mRNA which is then translated into a Tff2 protein in a cell. Mus musculus Tff2 is provided as SEQ ID NO: 89 and the amino acid sequence is SEQ ID NO: 91. Human TFF2 RNA is provided as SEQ ID NO: 90 and the amino acid sequence is SEQ ID NO: 92. Sequences having at least 95%, 96%, 97%, 98%, 99% sequence identity to SEQ ID NO: 89 or SEQ ID NO: 90 are also provided. Sequences having at least 95%, 96%, 97%, 98%, 99% sequence identity to SEQ ID NO: 91 or SEQ ID NO: 92 are also provided.

The terms “polynucleotide” or “nucleic acid” or “nucleic acid sequence” are used interchangeably herein and refer to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. Polynucleotide sequences provided herein are provided as the cDNA encoding for the lncRNA-ANRIL of interest or Tff2 protein.

As used herein, a “therapeutic” agent (e.g., a therapeutic polypeptide, nucleic acid, or transgene) is one that provides a beneficial or desired clinical result, such as the exemplary clinical results described herein. As such, a therapeutic agent may be used in a treatment as described herein. In some embodiments, the therapeutic agent is a construct, a viral vector or a composition comprising a construct of viral vector and comprises a polynucleotide capable of producing an ANRIL RNA or a Tff2 protein or combination thereof.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide. Similarly, a cellular sequence (e.g., a gene or portion thereof) that is incorporated into a viral vector is a heterologous nucleotide sequence with respect to the vector. The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as lncRNA, miRNA, siRNA, or shRNA. The transgene for use in the present invention is ANRIL (SEQ ID NO: 1-32) or APDC (SEQ ID NO: 33).

As used herein, the term “isolated” means that the material is removed from its original environment (e.g., the natural environment if it is naturally occurring). For example, a naturally occurring polynucleotide or polypeptide present in a living microorganism is not isolated, but the same polynucleotide or polypeptide, separated from some or all of the coexisting materials in the natural system, is isolated. Such polynucleotides could be part of a vector and/or such polynucleotides or polypeptides could be part of a composition and still be isolated in that such vector or composition is not part of its natural environment.

The present disclosure provides a construct comprising, consisting of, or consisting essentially of a heterologous promoter operably connected to a nucleic acid sequence encoding an ANRIL RNA sequence. The present disclosure further provides a construct comprising, consisting of, or consistent essentially of a heterologous promoter operably connected to a nucleic acid sequence encoding a TFF2 protein. A promoter is operably connected to a nucleic acid if the promoter mediates transcription of the nucleic acids to which it is connected. The terms “operably connected” and “operably linked” may be used interchangeably herein.

The terms “construct”, “vector” or “recombinant vector” are used interchangeably herein and refer to a synthetic nucleic acid that can be delivered into a host cell, either in vitro or in vivo. The vector can be a nucleic acid molecule capable of propagating another nucleic acid to which it is linked and include the term “expression vectors.” Constructs and vectors for use here include plasmids, viral vectors, BACs, YACs, transposons or any other form known to those of skill in the art. Constructs may be DNA or RNA. Vectors also include any pharmaceutical compositions thereof (e.g., a recombinant vector and a pharmaceutically acceptable carrier/excipient as provide herein). The term construct may include the vector as a self-replicating nucleic acid structure as well as the vector incorporated into the genome of a host cell into which it has been introduced. Constructs, including expression vectors, may comprise the nucleotide sequence encoding the ANRIL RNA of any one of the sequences described herein including SEQ ID NO:1-33 and a heterogeneous sequence necessary for proper propagation of the vector. Additionally or alternatively, constructs, including expression vectors, may comprise the nucleotide sequence encoding the TFF2 protein including any one of the nucleic acid sequences of SEQ ID NO: 89-90 or a sequence encoding one of SEQ ID NO: 91 or 92 and a heterogeneous sequence necessary for proper propagation of the vector. The heterogeneous sequence (i.e., sequence from a different species than the polynucleotide) can comprise a heterologous promoter or heterologous transcriptional regulatory region that allows for expression of the polynucleotide.

As used herein, the terms “heterologous promoter,” “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g., lncRNA, tRNA, or rRNA; also called “non-coding” RNA or “ncRNA”). Any promoters capable of expressing ANRIL RNA or TFF2 RNA and allowing translation of the Tff2 RNA into a protein in a cell are contemplated to be used in the practice of the present invention. In the Examples, a Cytomegalovirus immediate early (CMV IE) promoter was used. Other promoters known to those of skill in the art could be used such as the chicken 3-actin promoter. The promoter may also include an enhancer or an intron such as the chicken 3-actin intron to allow for increased expression. Expression of RNAs may also be via a polymerase III promoter or by linking the RNA to a tRNA which has an internal promoter to allow for expression of the RNA. Those of skill in the art will appreciate that a wide range of promoters are available for expression of an RNA or a protein and may include constitutive or inducible promoters or tissue-specific promoters as well.

In some embodiments, the nucleic acids (polynucleotides) of the ANRIL sequence are operably linked to a promoter. In some embodiments, the nucleic acids (polynucleotides) of the TFF2 sequence are operably linked to a promoter. The promoter can be a constitutive, inducible, or repressible promoter. Preferably, the promoter is capable of expression of the isoform encoded in the polynucleotide in the target cell. Exemplary promoters include, but are not limited to, the cytomegalovirus (CMV-IE) immediate early promoter, CAG (cytomegalovirus early enhancer element and chicken beta-actin) promoter, and PGK (Phosphoglycerate kinase) promoter.

As used herein, a promoter is “operably connected to” or “operably linked to” when it is placed into a functional relationship with a second polynucleotide sequence. For instance, a promoter is operably connected to a polynucleotide if the promoter is connected to the polynucleotide such that it may affect transcription of the polynucleotide coding sequence. In various embodiments, the polynucleotides may be operably linked to at least 1, at least 2, at least 3, at least 4, at least 5, or at least 10 promoters.

The promoter may be a tissue-specific promoter that drives gene expression in gingival epithelial cells, bone marrow stromal cells (BMSCs), osteoblasts (OBs), or bone marrow macrophages (BMMs). Alternatively, the promoter may be a constitutive promoter that is not tissue-specific. Use of such promoters may be advantageous when a high-level of gene expression is desirable. For example, the cytomegalovirus (CMV) immediate early (IE) promoter is commonly included in vectors used to genetically engineering mammalian cells, as it is well-characterized as a strong constitutive promoter (Boshart et al., Cell, 41:521-530 (1985)).

The term “sequence identity” as used herein refers to the extent that sequences are identical on a nucleotide-by-nucleotide basis or an amino acid-by-amino acid basis over a window of comparison. Nucleic acid and protein sequence identities can be evaluated by using any method known in the art. For example, the identities can be evaluated by using the Basic Local Alignment Search Tool (“BLAST”). The BLAST programs identity homologous sequences by identifying similar segments between a query amino or nucleic acid sequence and a test sequence which is preferably obtained from protein or nuclei acid sequence database. The BLAST program can be used with the default parameters or with modified parameters provided by the user.

The term “percentage of sequence identity” is calculated by comparing two optimally aligned sequences over the window of comparison, determining the number of positions at which the identical nucleic acid base (e.g., A, T, C, G) or the identical amino acid residue (e.g., Ala, Pro, Ser, Thr, GIy, VaI, Leu, lie, Phe, Tyr, Trp, Lys, Arg, His, Asp, GIu, Asn, GIn, Cys and Met) occurs in both sequences to yield the number of matched positions, dividing the number of matched positions by the total number of positions in the window of comparison (i.e., the window size), and multiplying the result by 100 to yield the percentage of sequence identity.

The term “substantial identity” of polynucleotide or polypeptide sequences means that a polynucleotide or polypeptide comprises a sequence that has at least 90%, 95% sequence identity to the polynucleotide or polypeptide of interest described herein. Alternatively, percent identity can be any integer from 95% to 100%. In one embodiment, the sequence identity is at least 95%, alternatively at least 99%. More preferred embodiments include at least: 96%, 97%, 98%, 99% or 100% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

In some preferred embodiments, the term “substantial identity” of polynucleotide or amino acid sequences for purposes of this invention means polynucleotide or polypeptide sequence identity of at least 95%, preferably 98%, most preferably 99% or 100%. Preferred percent identity of polynucleotides or polypeptides can be any integer from 95% to 100%. More preferred embodiments include at least 96%, 97%, 98%, 99%, or 100%.

### Viral Vectors

A viral vector comprising, consisting of, or consisting essentially of the constructs and viral components necessary to transfer the polynucleotides into a cell are also provided. The viral vector may comprise a polynucleotide comprising a heterologous promoter operably connected to a sequence encoding an ANRIL RNA selected from the group consisting of SEQ ID NO: 1-SEQ ID NO: 32 and portions or combinations thereof. The viral vector may comprise a polynucleotide comprising a heterologous promoter operably connected to a sequence encoding a TFF2 protein. The polynucleotide encoding the Tff2 protein is selected from the group consisting of SEQ ID NO: 89-SEQ ID NO: 90 or a polynucleotide encoding SEQ ID NO: 91-92 and portions or combinations thereof.

Introduction or transduction of the viral vector into a host cell allows for the expression of the encoded ANRIL RNA or the encoded TFF2 protein within the host cell. Methods of preparing, developing and packaging the viral vectors into virions (i.e., particles) are known in the art. In a preferred embodiment, the viral vector is an adeno-associated virus (AAV). It is understood that other gene delivery viral vectors, including retroviruses, lentiviruses, HSV vectors, or Semliki-Forrest-Virus vectors and adenoviruses may also be used in the context of the present invention. AAV vectors can generally be concentrated to titers of about 10¹⁴ viral particles per ml, a level of vector that has the potential to transduce a greater number of target cells, e.g., gingival cells, in a patient. Moreover, AAV-based vectors have a well-established record of safety and do not integrate at significant levels into the target cell genome, thus avoiding the potential for insertional activation of deleterious genes or deactivation of necessary genes. Accordingly, in certain embodiments the viral vector comprises an AAV vector.

In some embodiments, the expression cassette of the construct is flanked on the 5′ and 3′ end by at least one functional AAV ITR sequences. By “functional AAV ITR sequences” it is meant that the ITR sequences function as intended for the rescue, replication and packaging of the AAV virion. See Davidson et al., PNAS, 2000, 97(7)3428-32; Passini et al., J. Virol., 2003, 77(12):7034-40; and Pechan i., Gene Ther., 2009, 16:10-16, all of which are incorporated herein in their entirety by reference. For practicing some aspects of the present disclosure, the recombinant vectors comprise at least all of the sequences of AAV essential for encapsidation and the physical structures for infection by the rAAV. AAV ITRs for use in the vectors of the present disclosure need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810.

Use of any AAV serotype is considered within the scope of the present disclosure. In some embodiments, a rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV ITRs or the like. In some embodiments, the nucleic acid in the AAV comprises an ITR of AAV ITRs are AAV1, AAV2, AAV3, AAV4, AAV5, AAV6, AAV7, AAV8, AAVrh8, AAVrh8R, AAV9 (Aschauer et al., 2013), AAV10, AAVrh10, AAV11, AAV12, AAV2R471A, AAV DJ, a goat AAV, bovine AAV, or mouse AAV or the like. In certain embodiments, the nucleic acid in the AAV comprises an AAV9 ITR. In some embodiments, a vector may include a stuffer nucleic acid. In some embodiments, the stuffer nucleic acid may encode a fluorescent protein. In the Examples, AAV9 was used.

Numerous methods are known in the art for production of viral vectors, including rAAV vectors, including transfection, stable cell line production, and infectious hybrid virus production systems. Some of those systems include, but are not limited to, for example, adenovirus-AAV hybrids, herpesvirus-AAV hybrids (Conway, J E et al., (1997) J. Virology 71(11):8780-8789) and baculovirus-AAV hybrids.

### Pharmaceutical Compositions:

The present disclosure further provides a pharmaceutical composition. The pharmaceutical composition may comprise, consist essentially of or consists of the constructs encoding ANRIL RNA or the viral vectors encoding ANRIL RNA, and a pharmaceutically acceptable carrier. Additional or alternatively, the pharmaceutical compositions may comprise, consist essentially of, or consist of the construct encoding TFF2, or the viral vectors encoding TFF2, and a pharmaceutically acceptable carrier. The pharmaceutical compositions provided herein may further contain buffers and/or pharmaceutically acceptable excipients and/or pharmaceutically acceptable carriers. As is well known in the art, pharmaceutically acceptable excipients and/or carriers are relatively inert substances that facilitate administration of a pharmacologically effective substance and can be supplied as liquid solutions or suspensions, as emulsions, or as solid forms suitable for dissolution or suspension in liquid prior to use. The pharmaceutically acceptable carrier may be selected based upon the route of administration desired. For example, an excipient can give form or consistency, or act as a diluent. Suitable excipients include but are not limited to stabilizing agents, wetting and emulsifying agents, salts for varying osmolarity, encapsulating agents, pH buffering substances, and buffers. Suitably the pharmaceutically acceptable carrier helps maintain the construct or viral particle integrity of the viral vector prior to administration, e.g., provide a suitable pH balanced solution. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N.J. 1991). The compositions can be sterilized by conventional, well known sterilization techniques prior to administration (e.g., filtration, addition of sterilizing agent, etc.).

The pharmaceutical compositions will be formulated based on the route of administration effective for treating the subject. The exact dosage is chosen by the individual physician in view of the patient to be treated. Dosage and administration are adjusted to provide sufficient levels of the active agent(s) or to maintain the desired effect. Additional factors which are taken into account include the severity of the disease state, e.g., extent of the condition, history of the condition; age, weight and gender of the patient; diet, time and frequency of administration; drug combinations; reaction sensitivities; and tolerance/response to therapy. For any active agent, the therapeutically effective dose can be estimated initially either in cell culture assays or in animal models, usually mice, rabbits, dogs, or pigs. The animal model is also used to determine a desirable concentration range and route of administration.

In some embodiments, the pharmaceutical compositions of the present disclosure are formulated for administration by subcutaneous injection. Although not required, the compositions may optionally be supplied in unit dosage form suitable for administration of a precise amount.

In other embodiments, the pharmaceutical compositions of the present disclosure are formulated for gingival injections. This includes but is not limited to regular sub-gingival injections.

### Methods of Treatment:

Method for treating of a subject in need of lncRNA-ANRIL RNA or TFF2 are also provided. The methods comprise administering an effective amount of the construct, viral vector or pharmaceutical compositions provided herein to the subject. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. The subject may be in need of treatment for a wound, inflammation, bone loss, periodontitis, diabetes, metabolic bone disorder or atherosclerosis. Administration of the constructs, vectors or compositions comprising the constructs or vectors may result in amelioration of the condition, such as closure and healing of the wound, a decrease in inflammation or the symptoms related to inflammation, an increase in bone regeneration or decrease in bone loss, reversal of atherosclerosis or stalling of additional plaque formation, amelioration of periodontitis or alleviation of symptoms of diabetes.

As used herein, a “subject” may be interchangeable with “patient” or “individual” and means an animal, which may be a human or non-human animal, in need of treatment. A “subject in need of treatment” may include a subject having a disease, disorder, or condition that is responsive to therapy with the compounds disclosed herein alone or in combination with another bioactive agent.

As used herein the term “effective amount” refers to the amount or dose of the compound, such as upon single or multiple dose administration to the subject, which provides the desired effect. An effective amount can be determined by the attending diagnostician, as one skilled in the art, using known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

The present invention provides methods for delivering a construct or viral vector to a cell or a tissue. These methods involve contacting the cell or the tissue with both the construct, composition or viral vector of the present invention. Cells may be contacted with the agent directly or indirectly in vivo, in vitro, or ex vivo. Contacting encompasses administration to a cell, tissue, mammal, patient, or human. Further, contacting a cell includes adding the construct, viral vector or composition to a cell culture or topically. Constructs or compositions may include or be combined with compositions that allow introduction or entry into the cell. Other suitable methods may include introducing or administering a construct, viral vector or composition to a cell, tissue, mammal, or patient using appropriate procedures and routes of administration as defined herein.

The provided methods include administering the construct, viral vector or composition using any amount and any route of administration effective for treating the subject. The exact dosage is chosen by the individual physician in view of the patient to be treated. Dosage and administration are adjusted to provide sufficient levels of the active agent(s) or to maintain the desired effect. Additional factors which are taken into account include the severity of the disease state, e.g., extent of the condition, history of the condition; age, weight and gender of the patient; diet, time and frequency of administration; drug combinations; reaction sensitivities; and tolerance/response to therapy. The therapeutically effective dose can be estimated initially either in cell culture assays or in animal models, usually mice, rabbits, dogs, or pigs. The animal model is also used to determine a desirable concentration range and route of administration. The present disclosure provides lncRNA-ANRIL and TFF2. AAV compositions with extended duration of action; such agents are amenable to less frequent dosing as the viral vector would be expected to produce the ANRIL RNA or TFF2 over an extended period of time by cells infected with the viral vector. In some embodiments, constructs or viral vectors encoding the lncRNA-ANRIL or TFF2. compositions provided herein are dosed no more frequently than once per day. In some embodiments, dosing is no more frequent than once every 2, 3, 4, 5, 6, 7, 8, 9, or 10 days. In some embodiments, dosing is no more frequent than twice per week, once per week, once every two weeks, once a month, or once every 6 months, once a year or even only one time.

### Methods of Treating Periodontal Disease:

In some embodiments, the subject is need of a treatment for periodontitis. Periodontitis, a common oral disease responsible for tooth loss, is characterized by periodontal inflammation and progressive alveolar bone destruction. Furthermore, periodontitis can trigger an increase in systemic inflammation, negatively impacting cardiovascular, endocrine, and respiratory systems. Twice as prevalent in diabetic patients as in non-diabetics, periodontitis is a common diabetes-associated complication. Type 2 diabetes (T2D)-associated periodontitis is particularly severe and refractory in many cases. Currently, T2D afflicts 40 million Americans, a number expected to increase as the American population ages and becomes more obese. Periodontal inflammation not only stimulates osteoclastogenesis, but also interferes with the coupling of bone formation and bone resorption. T2D-associated periodontitis is associated with supernormal osteoclastogenesis with increased production of pro-inflammatory molecules like interleukin 1p (IL-10), IL-6, and CC chemokine ligand 2 (CCL2) leading to upregulated production of receptor activator of nuclear factor KB ligand (RANKL) by osteoblasts resulting in osteoclastogenesis and increased numbers of osteoclasts in periodontal tissues. Periodontal disease can trigger general systemic inflammation, adversely influencing cardiovascular, neural, reproductive, and endocrine systems

As demonstrated in the Examples, lncRNA-ANRIL and TFF2 ameliorates the severity of bone loss in periodontitis and, more particularly, T2D-associated periodontitis. lncRNA-ANRIL and TFF2 can be delivered via gingival injection, or otherwise directed to gingival epithelial cells.

### Methods of Treating Metabolic Bone Disorders and Inflammation:

The present disclosure further comprises methods of treating metabolic bone disorders and inflammation. The administration of the constructs, viral vectors and compositions provided herein to treat these conditions may include subcutaneous, intramuscular, or intravenous, or intraarticular injection. The metabolic bone disorders may include but are not limited to osteoporosis, osteomalacia, and hyperparathyroidism. Some symptoms related to metabolic bone disorders include, but are not limited to, inflammation and bone loss.

In some embodiments, the subject is in need of a treatment for inflammation. Inflammation refers to the release of pro-inflammatory cytokines from immune-related cells and the activation of the innate immune system. The tissue inflammatory response caused by bacteria, trauma, toxins, heat, or other factors, can potentially activate the innate immune responses. Innate immune responses are capable of not only combating infectious microbes but also contributing to pathological situations, such as sepsis, obesity, atherosclerosis, autoimmunity, osteoarthritis, and cancer.

In some embodiments, the subject has chronic inflammation. Chronic inflammation refers to a slow, long-term inflammation lasting several months to years. Chronic inflammation may be associated with a number of inflammation-mediated disorders, conditions, or diseases, such as diabetes, cardiovascular disease, artherosclerosis, arthritis and joint diseases, allergies, and chronic obstructive pulmonary disease. Risk factors that contribute to chronic inflammation include, obesity, age, diet, smoking, low sex hormones, stress, and sleep disorders.

In other embodiments, the subject has acute inflammation. Tissue damage due to trauma, microbial invasion, or noxious compounds can induce acute inflammation. Acute inflammation may start rapidly, become severe in a short time, and symptoms may last for hours, days, or a few weeks, e.g., 1-6 weeks. In some embodiments, the subject may have an infection, such as a viral or bacterial infection, or suffer from a condition such as sepsis, septic shock, or endotoxemia.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### EXAMPLES

### Example 1

### Introduction

Long non-coding (lnc) RNAs over 200 nucleotides in length can present special expression patterns of biological functions in genome organization and life processes [1-4]. There is emerging evidence that lncRNAs regulate specific gene expression by competing with miRNA for the same binding site on mRNA, thus eliminating miRNA-induced repression and stabilizing the corresponding mRNA [5, 6]. In humans, lncRNA-ANRIL, also known as CDKN2BAS, is a genetic risk factor for atherosclerosis, coronary artery disease (CAD), myocardial infarction (MI), periodontitis (PD), diabetes and cancer [7-19]. Previous studies have demonstrated that ANRIL promotes angiogenesis in cerebral infarctions associated with diabetes mellitus through VEGF upregulation via activation of the NF-κB signaling pathway [20]. It is still unclear how regulating ANRIL results in cellular or physiological processes associated with bone and adipose tissue metabolism. No ANRIL exons were found in mouse, limited studies of rodent models on the basic biological disease and therapeutic mechanisms of ANRIL [21].

To further gain insight, we studied the mouse lncRNA-ANRIL ortholog, AK148321 (a 70 kb mouse interval on chromosome 4), which has 50% base pair similarity human to mouse [22, 23]. AK148321 inferred to be a genetic risk factor for atherosclerosis, as well PD, diabetes, and cancer, was coined lncR-APDC (atherosclerosis, periodontitis, diabetes, and cancer). Like ANRIL, the regulatory function of IncR-APDC is not yet well understood. As reported, ANRIL is closely related to inflammatory states and metabolic disorders [24-27]. For bone, the inflammatory state can cause osteoclastic-osteogenic imbalance, which may be related to the TLR4-mediated signaling pathway [28, 29]. Also, in our previous research, lysine (K)-specific demethylase 6B (KDM6B), a target of miR-99a, is distinctively regulated during osteogenic differentiation [30]. Based on this, we sought to further understand the mechanism of IncR-APDC regulation of bone and adipose tissue metabolic disorders with inflammatory state.

Our goal is to gain a better understanding of the mechanistic regulatory role of IncR-APDC in bone and adipose tissue metabolism. AK148321 was confirmed to be severely reduced in these chr4 Δ70 kb/70 kb mice23. Previously, chr4 Δ70 kb/70 kb mice have been utilized to study regulatory features of AK148321 during development and in vascular diseases [31]. Chr4 Δ70 kb/70 kb mice were effectively utilized to investigate the functions of ANRIL. In our laboratory, using the chr4 Δ70 kb/70 kb mouse strain, we designed an APDC-knockout (APDC-KO) mouse. In this paper, we identify the phenotypic bone and adipose tissue differences between APDC-KO and wild type mice. With deletion/overexpression of IncR-APDC, osteogenesis, adipogenesis, osteoclastogenesis, molecular signaling pathway and mechanism of osteogenic function were explored. The data suggest that lncR-APDC acts for an important regulatory element in bone and fat tissue metabolism that has therapeutic possibility for treating bone disorders of which metabolism is disturbed.

### Materials and Methods

### APDC-KO Mouse Model and Genotyping

The mouse model in this study obtained from the Mutant Mouse Resource & Research Center (MMRRC) is supported from the National Institutes of Health. To create this model, lncRNA AK148321 was deleted in W4/129S6 ES cells through Cre/lox P recombination. AK148321 was confirmed to be severely reduced in these chr4 Δ70 kb/70 kb mice [23]. APDC-KO mice enrolled in this study underwent confirmational genotyping. To further confirm the APDC-null genotypes of this strain, tail DNA samples were extracted by DNeasy Blood & Tissue Kits, and genomic DNA genotyping analysis (QIAGEN, MD, USA) was performed with standard gDNA qPCR (primers found in Table 1) and gel electrophoresis was performed. All animals with the same genotype and same gender were randomized to the groups.

TABLE 1
Primers utilized for APDC-KO genotyping.

| SEQ ID NO | Nucleotide Sequence (5′ - 3′) |
|---|---|
| 34 | AAGGTATCCTAAATACTGTCTTCTTGCAG |
| 35 | CGAGTCAATTTTCTTCATGTTTATCCTCCA |
| 36 | CGTAATACGACTCACTATAGGGC |
| 37 | TATGAAAGCACACTTGTGGGCGTGT |

### Plasmid Transfection

To gain overexpression of lncR-APDC in bone marrow stromal cells (BMSCs), osteoblasts (OBs), and bone marrow macrophages (BMMs), a plasmid vector was constructed with a lncR-APDC (SEQ ID NO: 33) insert. The integrity of the vector was confirmed by digestion with KpnI-NotI, sequenced to confirm the correct DNA orientation and sequence (State Key Laboratory of Oral Disease, Sichuan University, Chengdu, China). To complete transfection, BMSCs, OBs and BMMs were transfected with the plasmid vector using lipofectamine 3000 reagents (Thermal Fisher Scientific, MA, USA). After transfected for twenty-four hours, harvested cells were analyzed to verify overexpression of IncR-APDC.

### Cell Culture and Cytological Staining

BMSCs, OBs and BMMs were isolated from 4-6 week old C57BL/6J, APDC-KO, and wild type (WT) mice. Isolated cells or transfected cells were plated in 10 cm dishes and maintained in αMEM with 10% FBS and 1% penicillin/streptomycin. Osteogenic differentiation of BMSCs/OBs was carried out using αMEM medium (A1049001, Gibco™, MT, USA) containing 50 g/ml ascorbic acid (Sigma-Aldrich, MO, USA), 10 mM b-glycerophosphate (Sigma-Aldrich) and 100 nM dexamethasone (Sigma-Aldrich). Adipogenic differentiation of BMSCs was carried out with insulin (0.5 mM) after treatment with dexamethasone (1 μM), 3-isobutyl-1-methylxanthine (IBMX) (0.5 mM) and insulin (0.5 mM) for 3 days. Osteoclastogenic differentiation of BMMs was processed with receptor activator of nuclear factor kappa B ligand (RANKL) (50 ng/ml) and macrophage-colony stimulating factor (M-CSF)-1 (10 ng/ml) following MSCF-1 pretreatment for 3 days. The human fetal osteoblasts (hFOBs) (ATCC® CRL-11372™, USA) were cultured in DMEM with 10% FBS and 1% penicillin/streptomycin with the osteogenesis-inducing medium.

Osteogenic, adipogenic and osteoclastogenic differentiation were analyzed via cytological staining (Alizarin red staining for osteogenesis (Sigma-Aldrich), Oil red O staining for adipogenesis (Sigma-Aldrich), trap staining for osteoclastogenesis (Sigma-Aldrich)).

RNA Extraction and Quantitative Real-Time Polymerase Chain Reaction (qRT-PCR)

Total RNA of the aforementioned cells was extracted with Quick-RNA Miniprep Kit (ZYMO Research, CA, USA). Total RNA from white adipose tissue (WAT) or bone tissues of WT/APDC-KO mice was extracted through using TRIzol reagent (Thermo Fisher Scientific, USA) with protocol as lysing samples, separating phases and isolating RNA. Isolated RNA was reverse-transcribed with the PrimeScript™ RT Reagent Kit with gDNA Eraser (Takara, Japan). Utilizing the SYBR-Green 2× Master Mix (Thermo Fisher Scientific, USA) for qRT-PCR. Total miRNAs of aforementioned cells were extracted via the miRNeasy Mini Kit (QIAGEN, MD, USA), and reverse-transcribed by the miRCURY LNA RT Kit (QIAGEN, MD, USA). The expression of miR-99a-5p was treated by miRCURY LNA SYBR Green PCR Kit (QIAGEN, MD, USA) for analyzing. The specific primers were listed in Table 2.

TABLE 2
primers used for qPCR (h for human species).

| | SEQ ID | |
|---|---|---|
| Name | NO | Sequence |
| U6 | 38 | Forward: 5′-CGCTTC GGCAGCACATATAC-3′ |
| | 39 | Reverse: 5′-TTCACGAATTTGCGTGTCAT-3′ |
| miR-99a-5p | 40 | MIMAT0000131: 5′AACCCGUAGAUCCGAUCUUGUG |
| | | (purchased from Qiagen (Cat. No. YP02119293) |
| KDM6B | 41 | Forward: 5′-TGAAGAACGTCAAGTCCATTGTG-3′ |
| | 42 | Reverse: 5′-TCCCGCTGTACCTGACAGT-3′ |
| lncR-APDC | 43 | Forward: 5′-CTTGCCCCTGCCTTCTTACT-3′ |
| | 44 | Reverse: 5′-GCTAAAGCCATTGAGTCG GC-3′ |
| ANRIL | 45 | Forward: 5′-GGACTACAGATGCACCACCAT-3′ |
| | 46 | Reverse: 5′-TGAGCACTGTGTCCATAGCA-3′ |
| hRunx2 | 47 | Forward: 5′-TGGTTACTGTCATGGCGGGTA-3′ |
| | 48 | Reverse: 5′-TCTCAGATCGTTGAACCTTGCTA-3′ |
| hGAPDH | 49 | Forward: 5′-AGCCACATCGCTCAGACAC-3′ |
| | 50 | Reverse: 5′-GCCCAATACGACCAAATCC-3′ |
| GAPDH | 51 | Forward: 5′-AGGTCGGTGTGAACGGATTTG-3′ |
| | 52 | Reverse: 5′-TGTAGACCATGTAGTTGAGGTCA-3′ |
| ALP | 53 | Forward: 5′-GCCTTTGAGGTTTTTGGTCA-3′ |
| | 54 | Reverse: 5′-AACCCAGACACAAGCATTCC-3′ |
| OSX | 55 | Forward: 5′-ATCTGACTTTGCTCCCCTTAACC-3′ |
| | 56 | Reverse: 5′-GGGCCCTGGTTGCAAGA-3′ |
| Runx2 | 57 | Forward: 5′-AACGATCTGAGATTTGTGGGC-3′ |
| | 58 | Reverse: 5′-CCTGCGTGGGATTTCTTGGTT-3′ |
| OCN | 59 | Forward: 5′-GCCGGAGTCTGTTCACTACC-3′ |
| | 60 | Reverse: 5′-GCGCTCTGTCTCTCTGACCT-3′ |
| BMP2 | 61 | Forward: 5′-GAGAAAAGCGTCAAGCCAAAC-3′ |
| | 62 | Reverse: 5′-GGTGCCACGATCCAGTCATT-3′ |
| C/EBPα | 63 | Forward: 5′-GACCATTAGCCTTGTGTGTACTGTATG-3′ |
| | 64 | Reverse: 5′-TGGATCGATTGTGCTTCAAGTT-3 |
| UCP1 | 65 | Forward: 5′-ACTGCCACACCTCCAGTCATT-3′ |
| | 66 | Reverse: 5′- CTTTGCCTCACTCAGGATTGG-3′ |
| Zic1 | 67 | Forward: 5′-CTGTTGTGGGAGACACGATG-3′ |
| | 68 | Reverse: 5′-CCTCTTCTCAGGGCTCACAG-3′ |
| MMP9 | 69 | Forward: 5′-GCAGAGGCATACTTGTACCG-3′ |
| | 70 | Reverse: 5′-TCCAGGTTATGGGCAGAGATT-3′ |
| CK | 71 | Forward: 5′-GAAGAAGACTCACCAGAAGCAG-3′ |
| | 72 | Reverse: 5′-TGATGTTATGATGGTCCCACTTG-3′ |
| BSP1 | 73 | Forward: 5′-GACTTTTGAGTTAGCGGCACT-3′ |
| | 74 | Reverse: 5′-CCG CCAGCTCGTTTTCATC-3′ |
| RANKL | 75 | Forward: 5′-CAGCATCGCTCTGTTCCTGTA-3′ |
| | 76 | Reverse: 5′-CTGCGTTTTCATGGAGTCTCA-3′ |
| APPL1 | 77 | Forward: 5′-GCTTTGTTAGAACCTCTACTGGG-3′ |
| | 78 | Reverse: 5′-GGTCAGGCAGATATAAAGGGTCA-3′ |
| APPL2 | 79 | Forward: 5′-CACCCTCACAGATTACACCAAC-3′ |
| | 80 | Reverse: 5′-GGAGAACCATAGTGTCTGCCAG-3′ |
| AdipoR1 | 81 | Forward: 5′-TCTTTTTGGGTGCAGTGCT-3′ |
| | 82 | Reverse: 5′-GCAATTCCTGAATAGTCCAGTT-3′ |
| AdipoR2 | 83 | Forward: 5′-GGGCATTGCAGCCATTAT-3′ |
| | 84 | Reverse: 5′-TAGGCCCAAAAACACTCCTG-3′ |
| APN | 85 | Forward: 5′-GCACTGGCAAGTTCTACTGCAA-3′ |
| | 86 | Reverse: 5′-GTAGGTGAAGAGAACGGCCTTGT-3′ |
| PPARγ | 87 | Forward: 5′-GTGCCAGTTTCGATCCGTAGA-3′ |
| | 88 | Reverse: 5′-GGCCAGCATCGTGTAGATGA-3′ |

### Western-Blot Analysis

Proteins of aforementioned cells or BMMs lysed through RIPA lysis buffer (Thermo Fisher Scientific, USA) were isolated by Sodium Dodecyl Sulfate polyacrylamide gel electrophoresis (SDS-PAGE) (Sigma-Aldrich), which were transferred to Polyvinylidene fluoride (PVDF) membranes. The membranes blocked with 5% skim milk for 2 h at room temperature, were incubated with primary antibodies overnight at 4° C. And then the membranes were washed with Tris-buffered saline with Tween (TBST) buffer. Secondary antibody was utilized to incubate the membranes at room temperature for 1 h. The results were visualized by Pierce ECL Western Blotting Substrate kit for enhanced chemiluminescence (ECL). Antibodies were listed in Table 3.

TABLE 3
Primary antibodies used for western blot. Secondary antibody
from Cell signaling Technology (7076, 7075, 1:10000)

| Name | Cat# & Business & Dilution |
|---|---|
| β-actin | 3700, Cell signaling Technology, 1:20000 |
| TLR4 | ab22048, Abcam, 1:1000 |
| MyD88 | 50010, Cell signaling Technology, 1:1000 |
| OPG | sc-390518, Santa Cruz Biotechnology, 1:1000 |
| p38 | 9216, Cell signaling Technology, 1:1000 |
| p-p38 | 9212, Cell signaling Technology, 1:1000 |

### Luciferase Assay

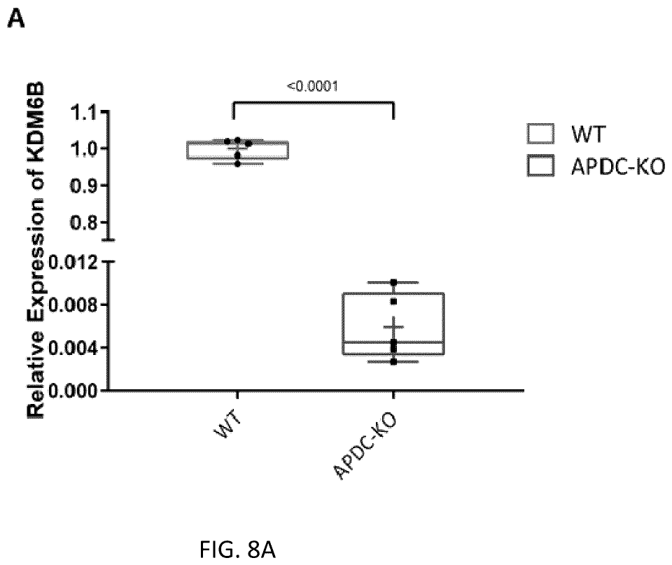

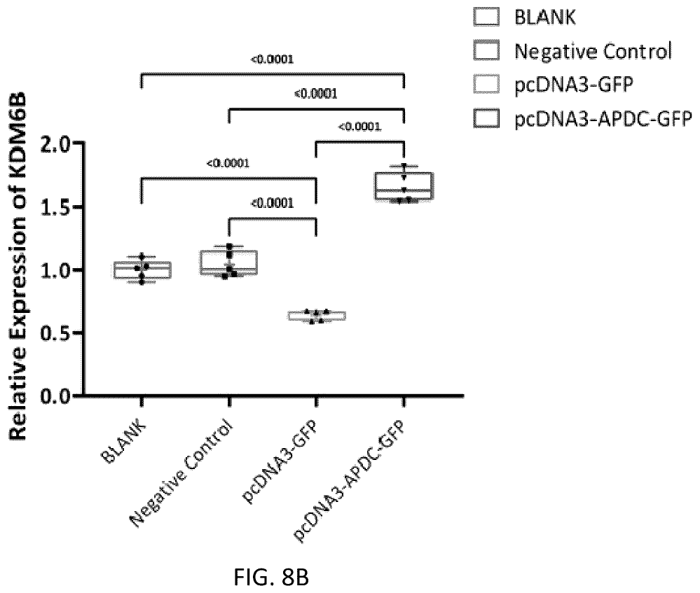

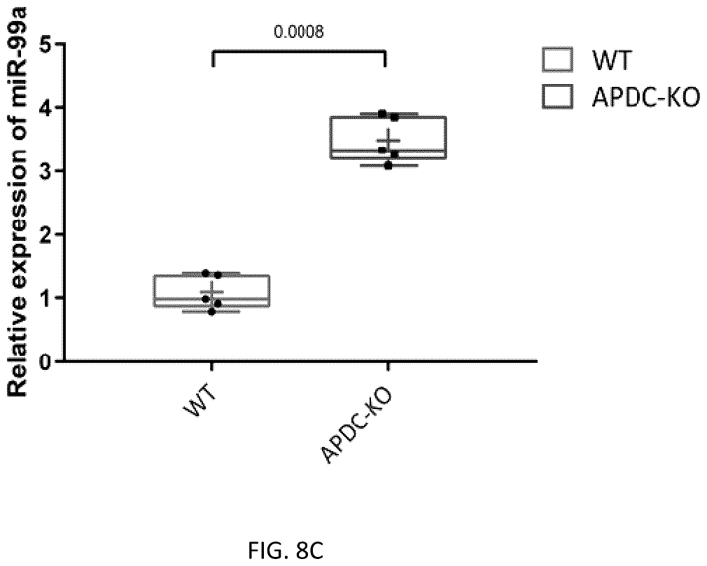

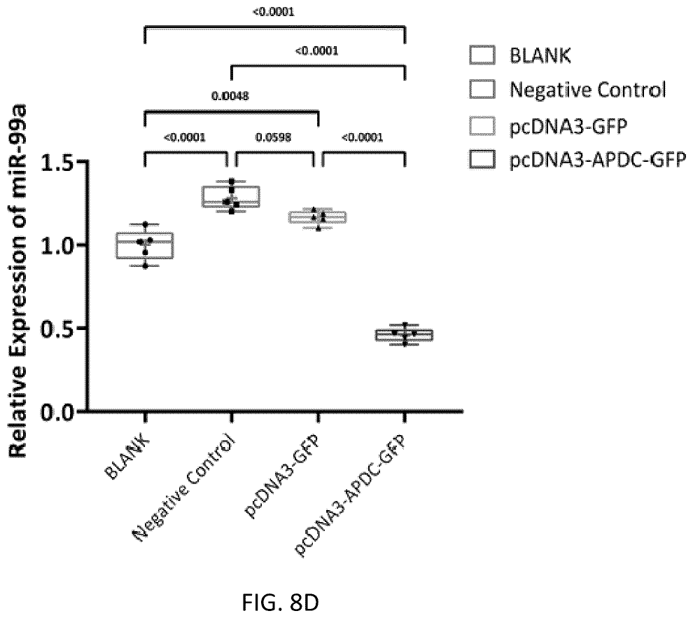

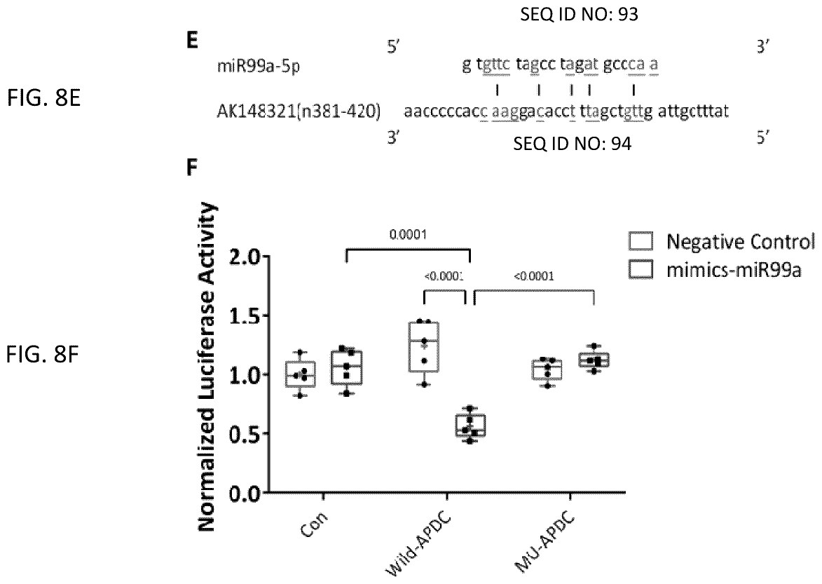

Luciferase reporter plasmids of wild type and mutant lncR-APDC were constructed by GENECFPS, China, Jiangsu. Site mutations in lncR-APDC were confirmed by using MicroInspector online software (bioinfo.uni-plovdiv.bg/microinspector) (FIG. 8E). Both fragments were confirmed by digestion of the plasmids with EcoRI-XhoI. All genetic material was sequenced to confirm the correct DNA orientation and sequence. BMSCs co-transfected with the pmirGLO Vector constructs, miRNA oligos used lipofectamine 3000 reagents (Thermal Fisher Scientific, MA, USA) for 24 h treatment. After transfection, harvested cells were analyzed for luciferase activity with the Dual-Glo® Luciferase Assay System (Promega). For each transfection, luciferase activity was averaged from five replicates.

### Histomorphometric and Immunohistochemical Staining of Bone and Adipose Tissues

For histological examination, tissue samples (7 for each group) were fixed in 10% formalin. Hard tissues were decalcified in 10% ethylenediaminetetraacetic acid (pH 7.0) for 2-3 weeks followed with dehydration in ethanol and embedded in paraffin wax. Sections were cut and mounted on glass slides with a thickness of 5 m. Three random slides from each sample were stained with hematoxylin and eosin. Immunohistochemical staining (IHC) was performed to detect osteocalcin (OCN) (primary antibody: ab93876, Abcam, 1:250) expression using a Histostain SP kit (Life Technologies). H&E and IHC staining were photographed with an OLYMPUS BX53 microscope. To prevents the introduction of examiner bias, every slide was coded.

### Statistical Analysis

All results were expressed as means±SD of three or more independent experiments. We used one-way analysis of variance and multivariate analysis of variance with Tukey post hoc test to test statistics significance via the software GraphPad Prism 9.1.2.

### Results

IncR-ANRIL and lncR-APDC Upregulation During Osteogenesis

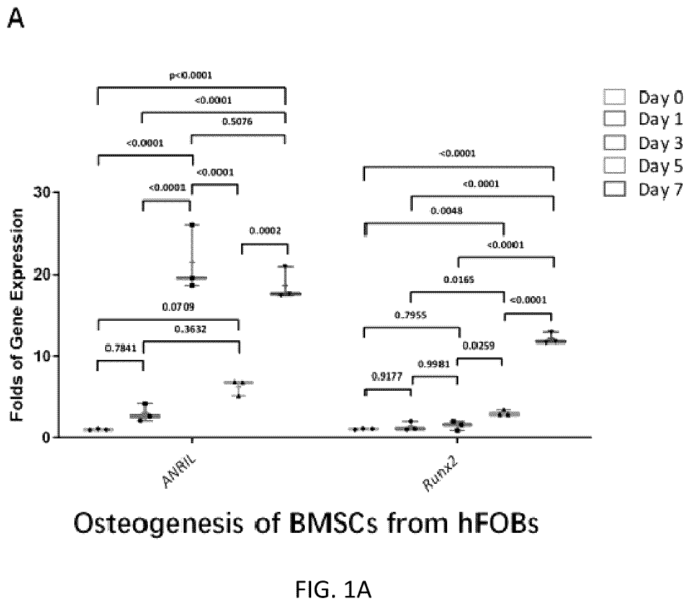

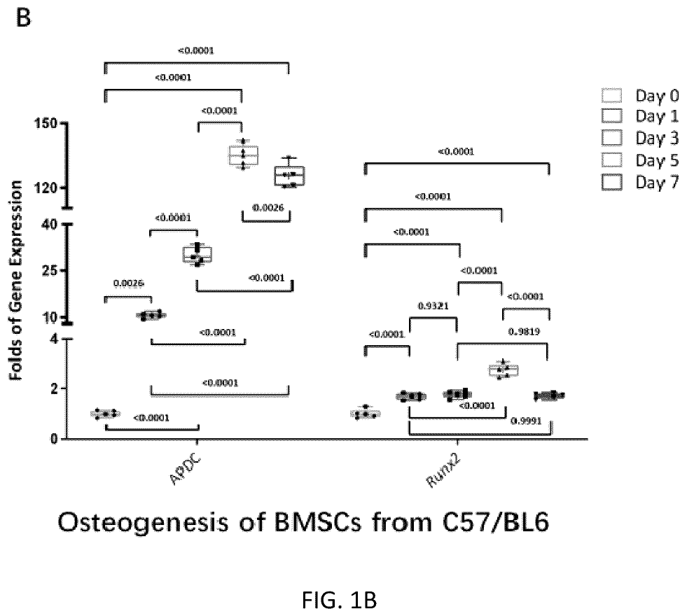

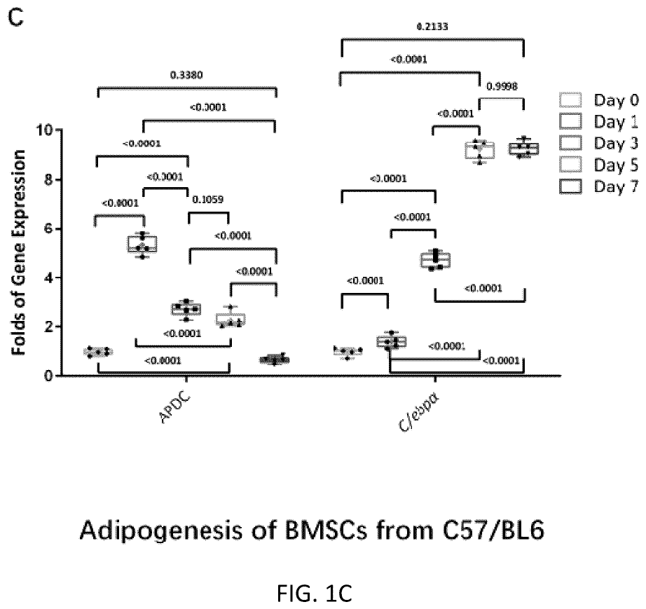

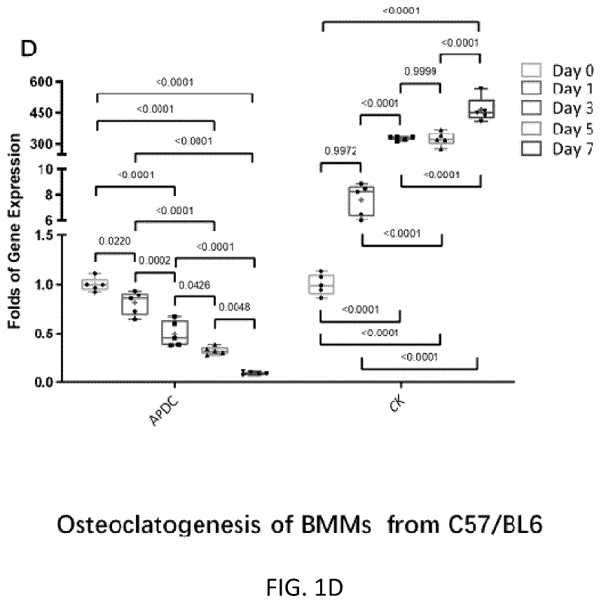

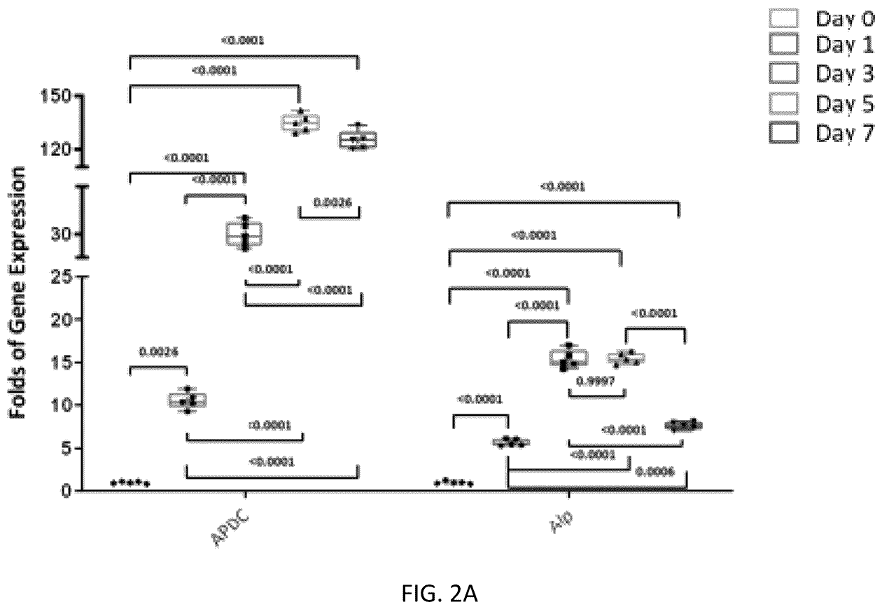

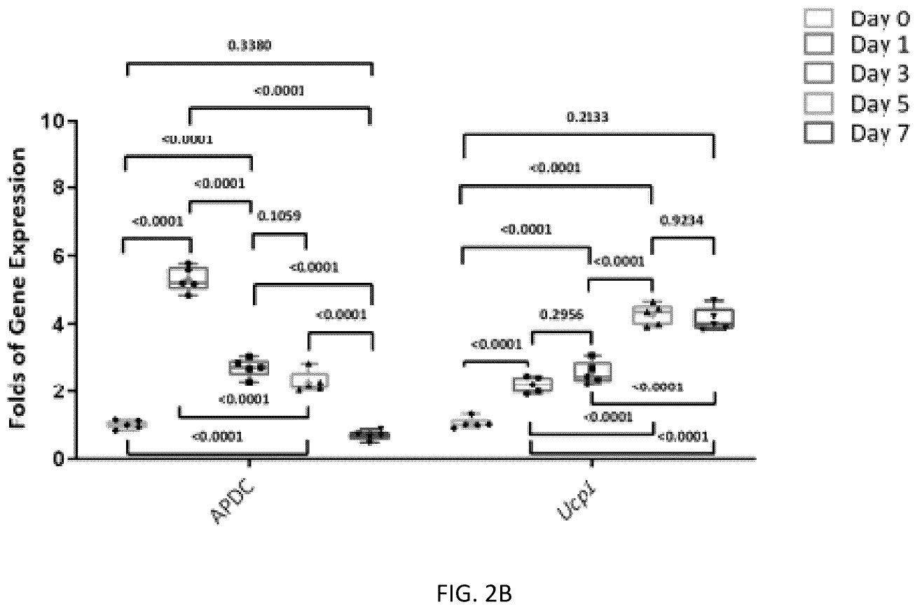

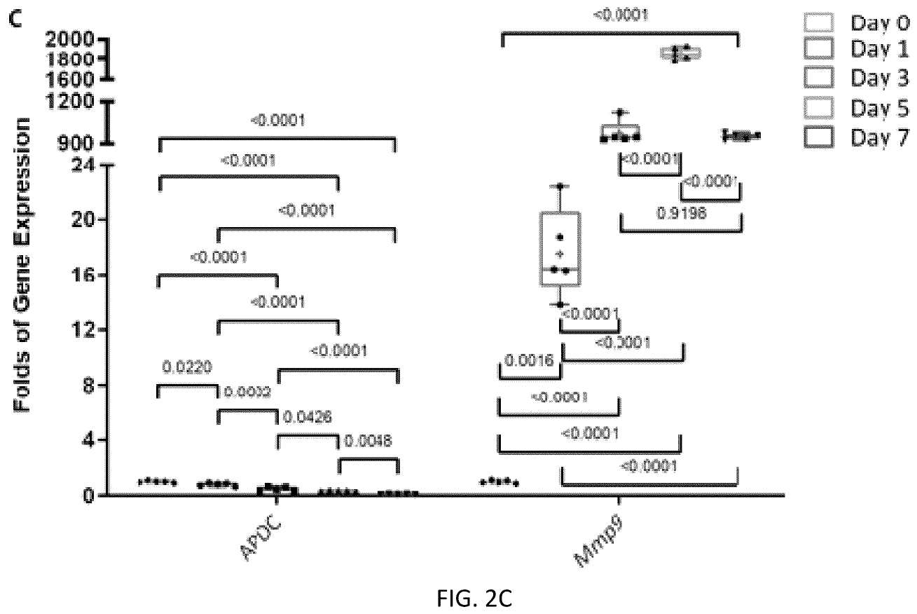

Upregulated expression of ANRIL in hFOBs was observed during osteogenesis (FIG. 1A). We harvested mBMSCs and mBMMs from C57/BL6 mice. The mBMSCs were induced to osteogenic differentiation and adipogenic differentiation. mBMMS were induced to osteoclastogenesis. mRNA expression of IncR-APDC and representative gene markers of osteogenesis, adipogenesis and osteoclastogenesis were analyzed via qPCR. We found that the expression of IncR-APDC in BMSCs increased significantly during the early stages of osteogenesis, while decreasing in adipogenesis and osteoclastogenesis (FIGS. 1B-1D and FIGS. 2A-2C). For the first time, we can predict that lncR-APDC has an impact on regulating stem cell and osteoclast differentiation, particularly on the processes involved in bone remodeling. Role of lncR-APDC in regulating stem cell differentiation and osteoclast differentiation

Deletion of lncR-APDC Impaired Osteogenesis while Promoting Adipogenesis and Osteoclastogenesis.

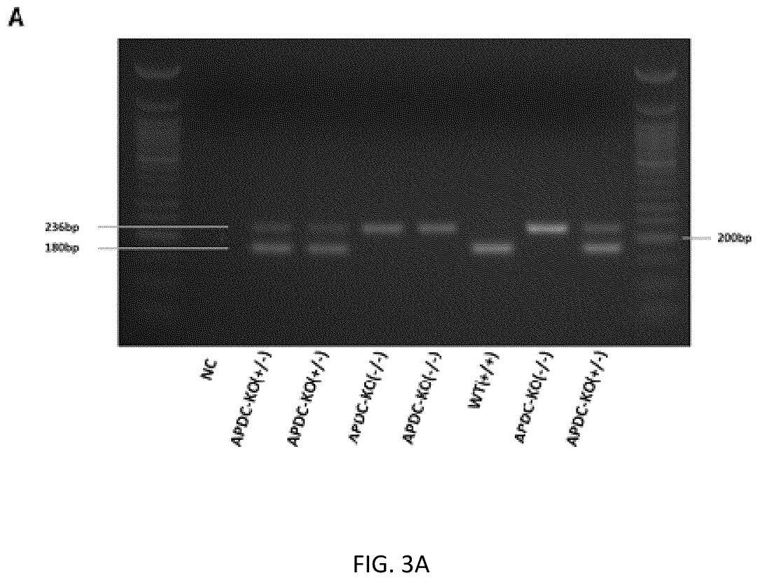

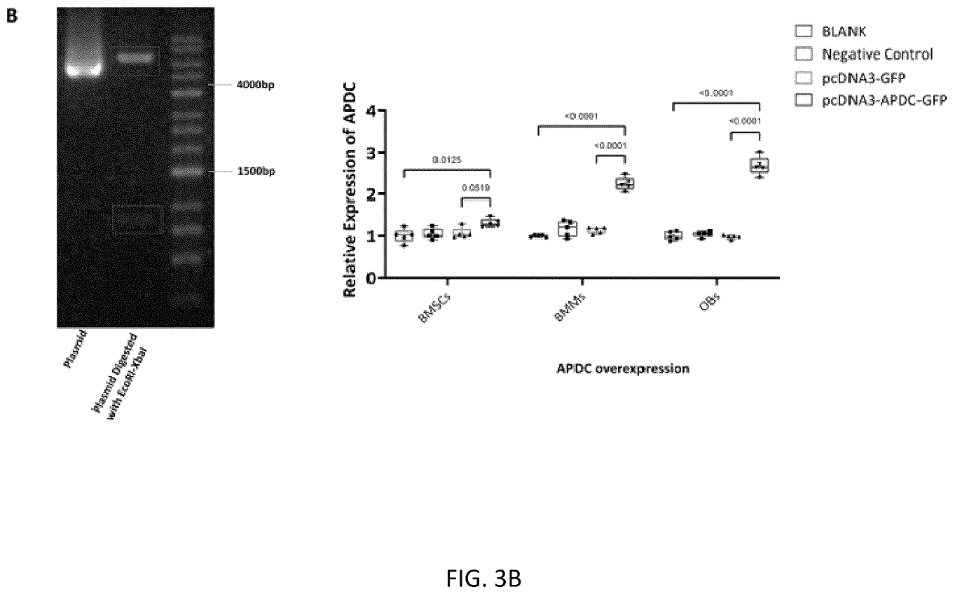

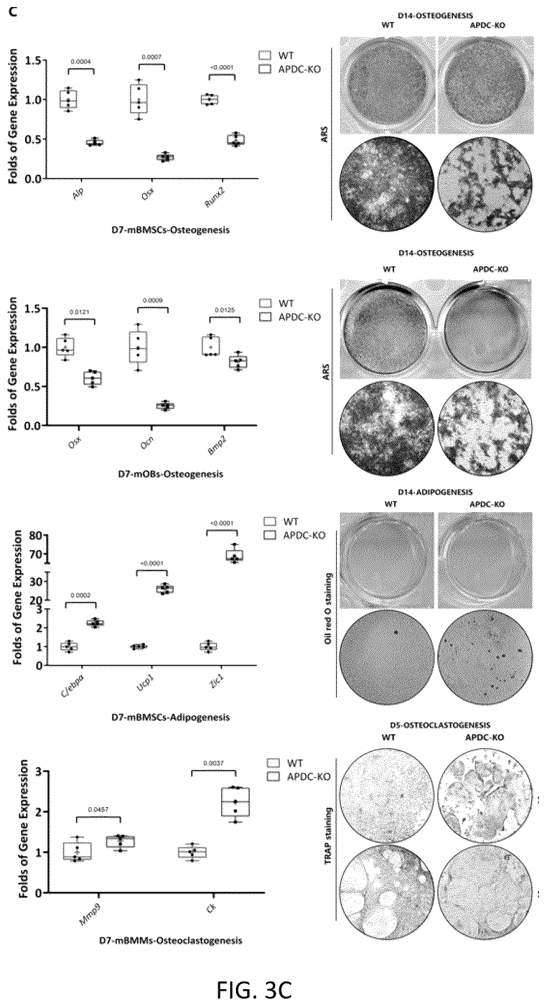

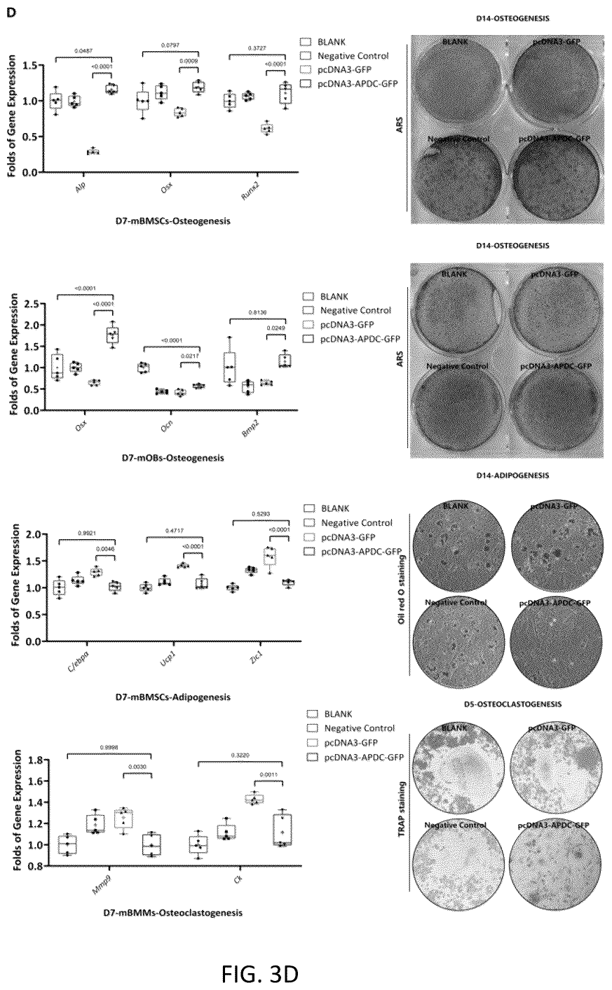

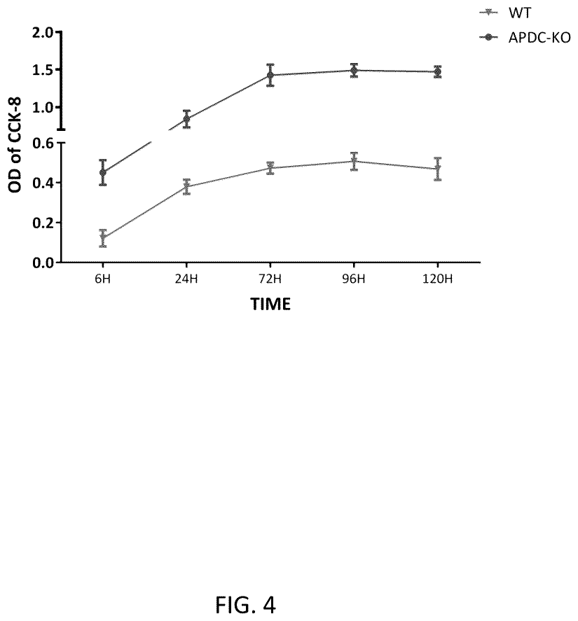

To investigate the role of IncR-APDC in BMSCs, OBs and BMMs, APDC-KO mice obtained from MMRRC were bred as described previously [12]. Genotyping results demonstrated successful deletion of IncR-APDC in the KO mice (FIG. 3A). Mice were separated into 2 groups: APDC-KO and WT. Excessive proliferation of BMSCs in the APDC-KO group was overserved (FIG. 4). Isolated BMSCs were cultured with osteogenesis- or adipogenesis-inducing media. In vitro studies included qPCR, Alizarin Red staining (ARS) and oil red O staining. Osteogenic, adipogenic and osteoclastogenic marker gene ALP, Osx, Runx2, Ocn, Bmp-2, C/EBPα, Ucp-1, Zic-1, Mmp-9 and cathepsin K (CatK) (primers found in Table 2) expression were compared between the WT and APDC-KO groups. Reduced osteogenesis and increased adipogenesis of BMSCs were demonstrated in the APDC-KO group by qPCR and staining analysis. The upregulation of UCP1 in the APDC-KO group demonstrates enhanced energy expenditure and mitochondrial activity of adipocytes and precursor adipocytes. A reduction in osteogenesis was observed in the APDC-KO group. Deletion of lncR-APDC enhanced osteoclastogenesis of BMMs as the expression of CatK was upregulated and TRAP-positive multinucleated cells (MNCs) were increased. (FIG. 3C).

Overexpression of IncR-APDC Promoted Osteogenesis while Inhibiting Adipogenesis and Osteoclastogenesis.

Overexpression of IncR-APDC obtained by plasmid transfection constructed via pcDNA3.1 cloning vector (GENECFPS, Jiangsu, China), was verified by DNA electrophoresis and qPCR in BMSCs, OBs and BMMs (FIG. 3B). The same comparisons as above were made between the BLANK, negative control, pcDNA3-GFP and pcDNA3-APDC-GFP groups. In vitro, the upregulation of osteogenesis of BMSCs and OBs was observed with overexpression of IncR-APDC compared with the control group. Adipogenesis was impaired in BMSCs with the overexpression of IncR-APDC. Overexpression of lncR-APDC inhibited osteoclastogenesis of BMMs. (FIG. 3D)

### Impaired Bone Formation, Altered Expression of Adiponectin & Receptors in APDC-KO Mice

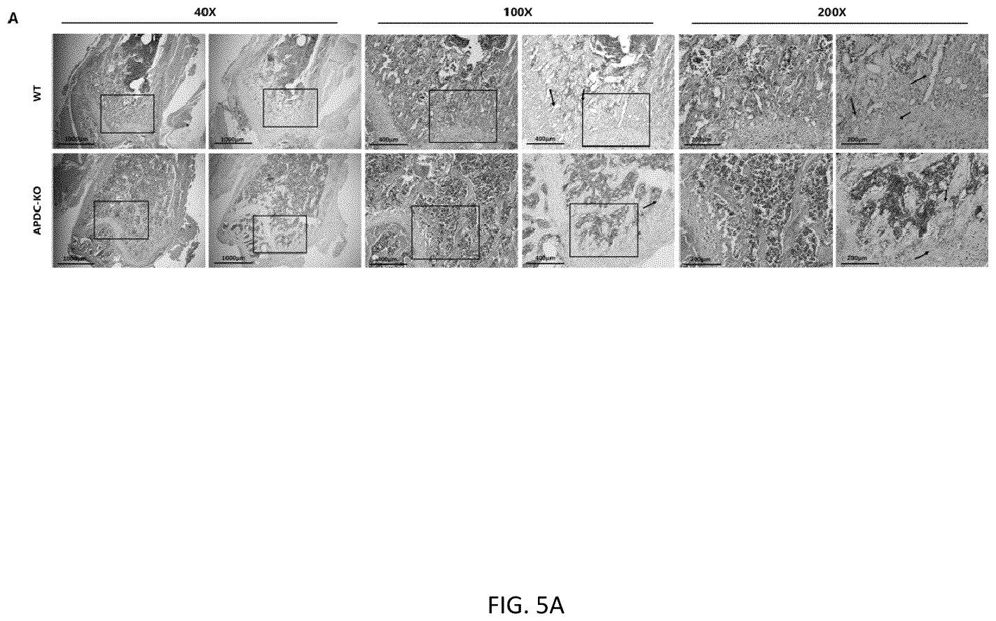

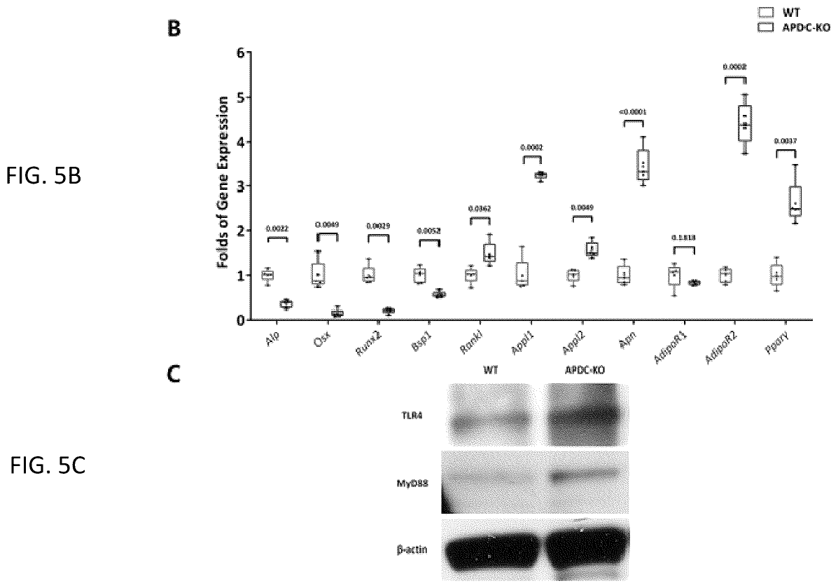

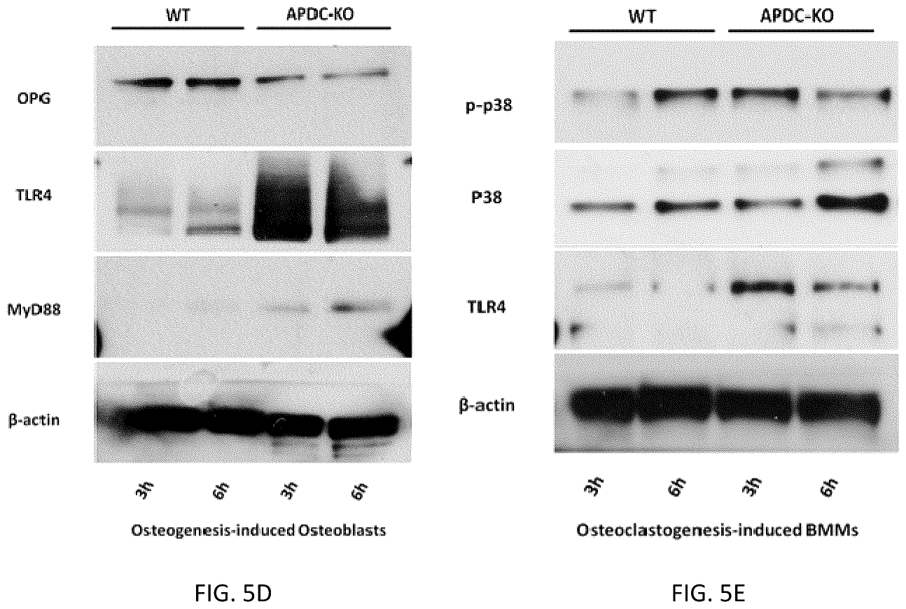

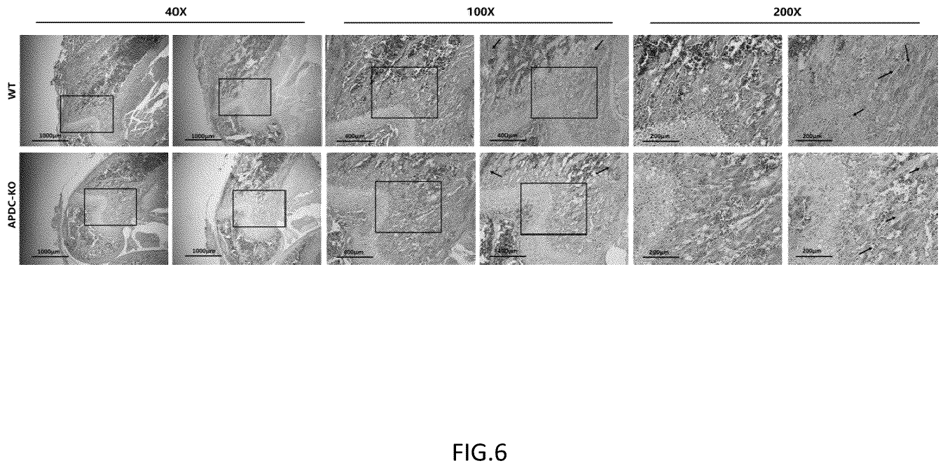

With OCN IHC staining, reduced OCN expression was observed in metaphyseal trabecular bone in the APDC-KO group of 8-week-old mice (FIG. 5A), which was similar to the results of 4-week-old (FIG. 6). mRNA expression of osteogenetic markers in bone tissues of APDC-KO and WT mice was analyzed by qPCR (FIG. 5B). Phenotypic differences include downregulated osteogenetic markers (Alp, Osx, Runx2, and Bsp1) and reduced bone mineralization in the APDC-KO group. Upregulation of RANKL also occurred in the APDC-KO group. These results suggest that deletion of IncR-APDC leads to overall decreased bone formation. The mRNA expression of adiponectin receptors 1/2 (AdipoR1/R2) and genes associated with adiponectin (APN) in the bone of both APDC-KO and WT mice are presented in FIG. 5B. Increased Pparγ, Appl1 and Appl2 appeared in the bones of the APDC-KO group compared to WT group. The expression of AdipoR1 was downregulated in the APDC-KO group, while the expression of AdipoR2 was upregulated.

No obvious differences in bone mass and microstructure of bone trabecula appeared around distal metaphysis in femurs with the HE staining, which was similar to the results of 8-week-old. While the reduced OCN expression was observed in metaphyseal trabecular bone in APDC-KO group of 4-week-old with OCN IHC staining.

To investigate signaling pathways involved with lncR-APDC in bone marrow (BM), markers of different signaling pathways were analyzed by western blot. TLR4/MyD88 signaling in BM is activated in lncR-APDC KO mice (FIG. 5C). Different signal pathways were investigated in osteogenesis-induced BMSCs and osteoclastogenesis-induced BMMs. Expression of TLR4 and MyD88 increased in BMSCs of APDC-KO group with time compared to WT group (FIG. 5D). Phosphorylation of p38 appeared earlier in BMMS from APDC-KO group compared to WT group (FIG. 5E), which indicates that lncR-APDC may be involved in the activation of the mitogen-activated protein kinase (MAPK) signal pathway through p-p38 in osteoclastogenesis.

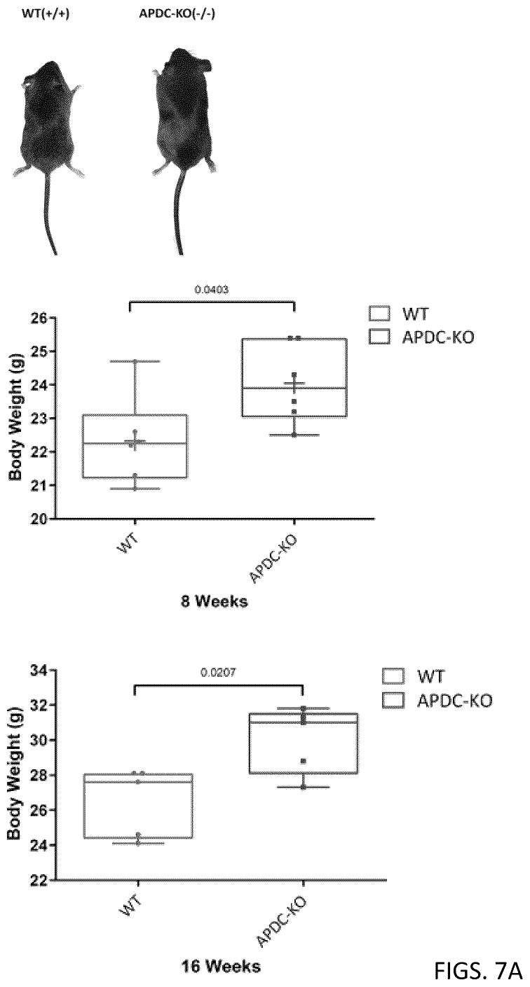

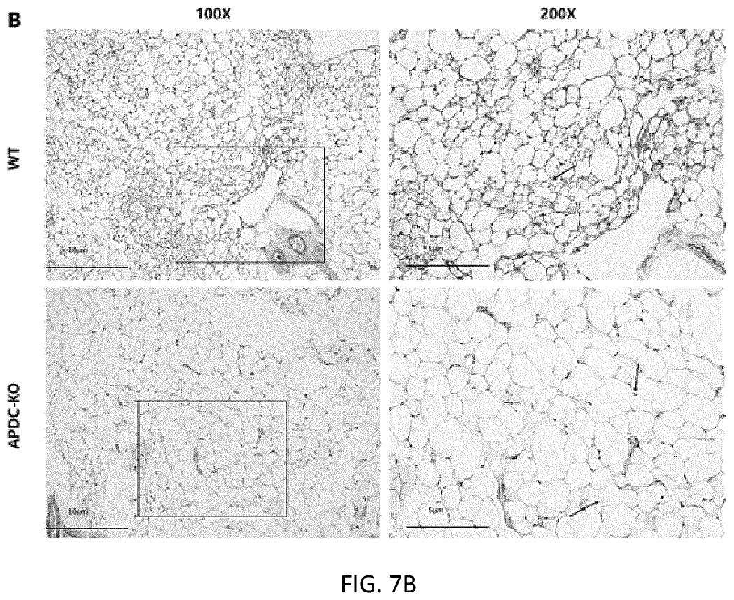

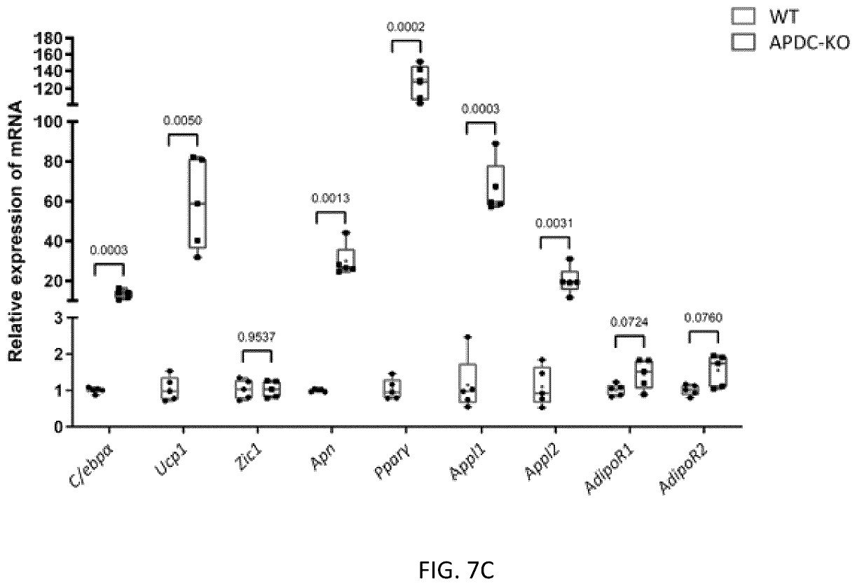

We observed that APDC-KO mice were generally bigger in size and heavier in weight compared to WT mice. (FIG. 7A). This led us to further examine the WAT distribution and the histomorphology in these mice. We found that adipocytes in WAT in the inguinal region showed increased size in lncR-APDC KO mice (FIG. 7B). We also focused on the changes of gene expression of WAT in 8-week-old male APDC-KO mice. With qPCR analysis, deletion of lncR-APDC enhanced adipogenic markers (C/EBPα, Ucp1, and Pparγ) in WAT, while molecules related to Apn were upregulated as well, such as Appl1/2 (FIG. 7C). However, the upregulation of AdipoR1/R2 expression was not obvious and there was no statistical significance. These results consistent with in vitro findings demonstrate that lncR-APDC suppresses adipogenesis and obesity.

IncR-APDC Affects Osteogenesis Through miR-99a/KDM6B

Our results revealed that, KDM6B was downregulated with the absence of IncR-APDC from BM cells. On the contrary, with overexpression of IncR-APDC, KDM6B was upregulated (FIG. 8A, FIG. 8B). In previous research, we discovered that KDM6B is a target of miR-99a, and is also specifically regulated by miR-99a during osteogenic differentiation30. We supposed that miR-99a might be a potential binding site of IncR-APDC. While miR-99a was upregulated with the absence of IncR-APDC, downregulated miR-99a was observed with overexpression of IncR-APDC (FIGS. 8C-8D). Using MicroInspector online software (bioinfo.uni-plovdiv.bg/microinspector) to predict the miRNA target site, lncR-APDC contains a single element complementary to miR-99a-5p (a putative target site located between n381 and n420 of IncR-APDC) (FIG. 8E). This indicates that the functional inhibition of miR-99a may be induced by binding IncR-APDC, which was proved by luciferase assay as follows. The mutant lncR-APDC (GENECFPS, Jiangsu, China) and wild type IncR-APDC inserted in Luciferase Vector were utilized in a luciferase assay to investigate whether miR-99a was a potential binding target of lnc-APDC. These vectors were transfected into BMSCs, while either control or miR-99a mimics co-transfected. The miR-99a mimics significantly suppressed luciferase activity, whereas mutated lncR-APDC effectively reversed this repression (FIG. 8F), suggesting that miR-99a is a direct binding target of lnc-APDC (FIG. 8G).

### Discussion

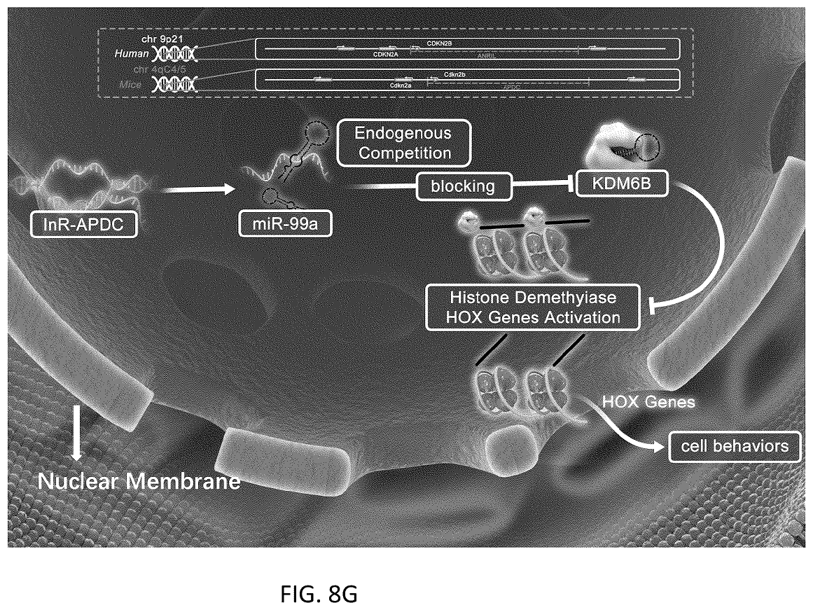

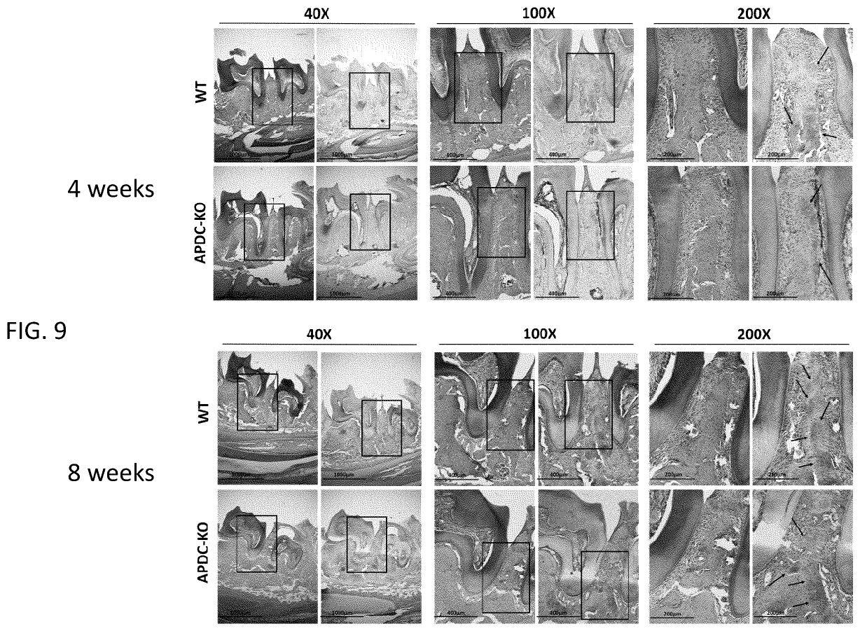

ANRIL (CDKN2BAS) has been confirmed as a genetic risk factor for atherosclerosis, periodontitis, diabetes, and cancer, which represent complicated biological functions and mechanisms [7-16, 20]. ANRIL knockout resulted in the repression of three genes ADIPOR1, VAMP3, and C11ORF10, associated with increased risk of CAD and PD [34]. As known, ANRIL regulates inflammatory responses by influencing glucose and fatty acid metabolism as well as the immune response [13, 17, 34]. LncR-APDC, an ortholog of IncR-ANRIL, is hypothesized to have similar biological functions to ANRIL, including important regulatory mechanisms in bone and adipose tissue metabolism [22, 23]. LncR-APDC and ANRIL exhibit the same promotion of osteogenesis in both mBMSCs and hFOBs. We report significant upregulation of IncR-APDC in the early stages of osteogenesis. IncR-APDC was downregulated in adipogenesis and osteoclastogenesis. The chr4 Δ70 kb/70 kb mouse, in which lncR-APDC is severely reduced, has been utilized as a model for investigating phenotype, biological functions and mechanisms, studying the regulating features of IncR-APDC during the development and vascular diseases [31]. With kinds of primary cells from APDC-KO mice, decreased presence of IncR-APDC leads to diminished osteogenesis by BMSCs and OBs and enhanced adipogenesis of BMSCs and osteoclastogenesis of BMMs. On the contrary, overexpression of IncR-APDC inhibits the adipogenic and osteoclastogenic lineages. Our results with APDC-KO mice suggest strong actions of IncR-APDC on osteogenesis, adipogenesis, and osteoclastogenesis. In the absence of IncR-APDC, osteogenic markers are remarkably downregulated, and significant inhibition of osteogenesis was observed in bone tissue of femurs. Except that, the more obvious OCN downregulation was especially observed in alveolar bone in APDC-KO group (FIG. 9). These results demonstrate the role of IncR-APDC in bone metabolism. With our displayed results, lncR-APDC is involved in the activation of MAPK signal pathways through p-p38 during osteoclastogenesis. As reported, MAPK/p-38 and nuclear factor kappa B (NFκB) are involved downstream of MCSF and RANKL inducing osteoclast differentiation and survival via cAMP response-element binding protein (CREB) phosphorylation [35-37]. Additionally, decreased presence of IncR-APDC activates TLR4/MyD88 signaling of osteoclasto-osteogenic balance, especially during the early stage of osteoclastic activation. Osteoclasto-osteogenic imbalance caused by inflammatory state or metabolic disorders is involved with the high expression of TLR4, and inhibition of TLR4/MyD88/NFκB-related signaling can inhibit osteoclastic activation and osteogenic destruction through different pathways [28, 29, 38, 39]. These indicate that osteoclasto-osteogenic imbalance is mediated with lncR-APDC by activated MAPK/p38 and TLR4/MyD88.

According to previous research, the regulatory mechanism categories of lncRNAs can be divided into interfering with the translation of an encoded gene, binding to functional proteins or chromosomes, facilitating post-transcriptional modification or precursor substance of small molecule RNA [2-4], and especially functioning as a competing endogenous RNA (ceRNA), competitively binding to the corresponding miRNA response elements (MRE) [5, 6, 40]. The downregulated KDM6B and upregulated miR-99a is proved with the IncR-APDC deficiency, while downregulation of miR-99a and upregulation of KDM6B were found with overexpression of IncR-APDC. The KDM6B gene has been confirmed as a target of miR-99a, which could affect the biological function of HOX genes, especially osteogenic differentiation of BMSCs [30]. Due to MicroInspector online software (bioinfo.uni-plovdiv.bg/microinspector), the miRNA target site predicted, lncR-APDC contains a single element complementary (a putative target site located between n381 and n420 of IncR-APDC) to miR-99a-5p. This indicates that the functional inhibition of miR-99a may be induced by binding IncR-APDC. Upon completion of a luciferase assay, miR-99a was identified as the direct binding target of IncR-APDC. This is the first reported investigation identifying the influence of osteogenic function by miR-99a through regulating the KDM6B levels. We infer that bone metabolism regulation is regulated through the IncR-APDC/miR-99a/KDM6B/Hox/Runx2 pathway.

Adipokines, the cell-signaling molecules secreted by adipose tissue, play functional roles in the crosstalk between bone and adipose tissue. In this study, we determined the enhancement of adipogenesis in the IncR-APDC deficiency mouse model. The gene expression of adiponectin (APN), AdipoRs, and APPLs was upregulated in WAT of APDC-KO mice, which indicates that the local adipogenesis and function of APN were enhanced. With these findings, for the first time, we demonstrated the positive correlation between lncR-APDC and APN/APN receptors. APN is a major adipokine with bone anabolic actions [41-43]. This correlation has vast potential for pharmacological treatment of bone metabolism diseases, especially diabetes. The research reported that adipogenesis could be inhibited by paracrine-secreted APN, while endocrine-secreted APN could decrease the levels of circulating bone-activating hormones [44]. Increasing APN expression in lncR-APDC deficiency might lead to the increased biological function of endocrine-secreted APN, thereby enhancing adipogenesis and diminishing osteogenesis in BM. Recent studies suggested that excessive accumulation of marrow adipocytes, caused by BMSCs, plays a key role in aging-related bone loss [45, 46]. In this study, the gain- and loss-of lncRNA-APDC models demonstrate that lncRNA-APDC modulates both the bone remodeling and the metabolic process of adipocytes. These indicate that lncRNA-APDC may be involved in the transdifferentiation of osteoblasts and adipocytes and has great potential preventive and/or therapeutic effects on bone and fat metabolic diseases, especially aging-related bone loss.

IncR-APDC represents an important regulatory element in bone and adipose tissue metabolism. The deletion of IncR-APDC impaired osteogenesis, while promoting adipogenesis and osteoclastogenesis. The overexpression of IncR-APDC stimulates osteogenesis but inhibits adipogenesis and osteoclastogenesis. LncR-APDC targets miR-99a and enhances osteogenesis through miR-99a/KDM6B/Hox/Runx2 pathways. It also can inhibit osteoclastogenesis through MAPK/p-38 and TLR4/MyD88 signaling pathways and regulate adipogenesis via APN-related pathways. IncR-APDC may prove to be a potential therapeutic target for bone and fat metabolic diseases.

### REFERENCES FOR EXAMPLE 1

1. Guttman M, Amit I, Garber M, French C, Lin M F, Feldser D, et al. Chromatin signature reveals over a thousand highly conserved large non-coding RNAs in mammals. Nature 2009; 458:223-7.
2. Geisler S, Coller J. RNA in unexpected places: long non-coding RNA functions in diverse cellular contexts. Nat Rev Mol Cell Biol 2013; 14:699-712.
3. Kung J T, Colognori D, Lee J T. Long noncoding RNAs: past, present, and future. Genetics 2013; 193:651-69.
4. Ponting C P, Oliver P L, Reik W. Evolution and functions of long noncoding RNAs. Cell 2009; 136:629-41.
5. Salmena L, Poliseno L, Tay Y, Kats L, Pandolfi P P. A ceRNA hypothesis: the Rosetta Stone of a hidden RNA language?Cell 2011; 146:353-8.
6. Tay Y, Kats L, Salmena L, Weiss D, Tan S M, Ala U, et al. Coding-independent regulation of the tumor suppressor PTEN by competing endogenous mRNAs. Cell 2011; 147:344-57.
7. Helgadottir A, Thorleifsson G, Manolescu A, Gretarsdottir S, Blondal T, Jonasdottir A, et al. A common variant on chromosome 9p21 affects the risk of myocardial infarction. Science (New York, N Y) 2007; 316:1491-3.
8. McPherson R, Pertsemlidis A, Kavaslar N, Stewart A, Roberts R, Cox D R, et al. A common allele on chromosome 9 associated with coronary heart disease. Science (New York, N Y) 2007; 316:1488-91.
9. Helgadottir A, Thorleifsson G, Magnusson K P, Grétarsdottir S, Steinthorsdottir V, Manolescu A, et al. The same sequence variant on 9p21 associates with myocardial infarction, abdominal aortic aneurysm and intracranial aneurysm. Nature genetics 2008; 40:217-24.
10. Schunkert H, Gotz A, Braund P, McGinnis R, Tregouet D A, Mangino M, et al. Repeated replication and a prospective meta-analysis of the association between chromosome 9p21.3 and coronary artery disease. Circulation 2008; 117:1675-84.
11. Holdt L M, Beutner F, Scholz M, Gielen S, Gabel G, Bergert H, et al. ANRIL expression is associated with atherosclerosis risk at chromosome 9p21. Arteriosclerosis, thrombosis, and vascular biology 2010; 30:620-7.
12. Harismendy O, Notani D, Song X, Rahim N G, Tanasa B, Heintzman N, et al. 9p21 DNA variants associated with coronary artery disease impair interferon-gamma signalling response. Nature 2011; 470:264-8.
13. Schaefer A S, Richter G M, Dommisch H, Reinartz M, Nothnagel M, Noack B, et al. CDKN2BAS is associated with periodontitis in different European populations and is activated by bacterial infection. Journal of medical genetics 2011; 48:38-47.
14. Congrains A, Kamide K, Oguro R, Yasuda O, Miyata K, Yamamoto E, et al. Genetic variants at the 9p21 locus contribute to atherosclerosis through modulation of ANRIL and CDKN2A/B. Atherosclerosis 2012; 220:449-55.
15. Deloukas P, Kanoni S, Willenborg C, Farrall M, Assimes T L, Thompson J R, et al. Large-scale association analysis identifies new risk loci for coronary artery disease. Nature genetics 2013; 45:25-33.
16. Hannou S A, Wouters K, Paumelle R, Staels B. Functional genomics of the CDKN2A/B locus in cardiovascular and metabolic disease: what have we learned from GWASs?Trends in endocrinology and metabolism: TEM 2015; 26:176-84.
17. Aarabi G, Zeller T, Heydecke G, Munz M, Schafer A, Seedorf U. Roles of the Chr.9p21.3 ANRIL Locus in Regulating Inflammation and Implications for Anti-Inflammatory Drug Target Identification. Front Cardiovasc Med 2018; 5:47.
18. Cho H, Li Y, Archacki S, Wang F, Yu G, Chakrabarti S, et al. Splice variants of lncRNA RNA ANRIL exert opposing effects on endothelial cell activities associated with coronary artery disease. RNA Biol 2020; 17:1391-401.
19. Chen S, Zhong H, Wang Y, Wang Z, Liang X, Li S, et al. The clinical significance of long non-coding RNA ANRIL level in diabetic retinopathy. Acta Diabetol 2020; 57:409-18.
20. Zhang B, Wang D, Ji T F, Shi L, Yu J L. Overexpression of lncRNA ANRIL up-regulates VEGF expression and promotes angiogenesis of diabetes mellitus combined with cerebral infarction by activating NF-κB signaling pathway in a rat model. Oncotarget 2017; 8:17347-59.
21. Kong Y, Hsieh C H, Alonso L C. ANRIL: A lncRNA at the CDKN2A/B Locus With Roles in Cancer and Metabolic Disease. Front Endocrinol (Lausanne) 2018; 9:405.
22. Karolchik D, Kuhn R M, Baertsch R, Barber G P, Clawson H, Diekhans M, et al. The UCSC Genome Browser Database: 2008 update. Nucleic acids research 2008; 36:D773-9.
23. Visel A, Zhu Y, May D, Afzal V, Gong E, Attanasio C, et al. Targeted deletion of the 9p21 non-coding coronary artery disease risk interval in mice. Nature 2010; 464:409-12.
24. Wan J, Bao Y, Hou L J, Li G J, Du L J, Ma Z H, et al. lncRNA ANRIL accelerates wound healing in diabetic foot ulcers via modulating HIF1A/VEGFA signaling through interacting with FUS. J Gene Med 2023; 25:e3462.
25. Kong Y, Sharma R B, Nwosu B U, Alonso L C. Islet biology, the CDKN2A/B locus and type 2 diabetes risk. Diabetologia 2016; 59:1579-93.
26. Biswas S, Coyle A, Chen S, Gostimir M, Gonder J, Chakrabarti S. Expressions of Serum lncRNAs in Diabetic Retinopathy—A Potential Diagnostic Tool. Front Endocrinol (Lausanne) 2022; 13:851967.
27. Loos B G, Van Dyke T E. The role of inflammation and genetics in periodontal disease. Periodontol 2000 2020; 83:26-39.
28. Liu Z, He Y, Xu C, Li J, Zeng S, Yang X, et al. The role of PHF8 and TLR4 in osteogenic differentiation of periodontal ligament cells in inflammatory environment. J Periodontol 2021; 92:1049-59.
29. Zeng X Z, Zhang Y Y, Yang Q, Wang S, Zou B H, Tan Y H, et al. Artesunate attenuates LPS-induced osteoclastogenesis by suppressing TLR4/TRAF6 and PLC71-Ca(2+)-NFATc1 signaling pathway. Acta pharmacologica Sinica 2020; 41:229-36.
30. Tang Y, Zhang L, Tu T, Li Y, Murray D, Tu Q, et al. MicroRNA-99a is a novel regulator of KDM6B-mediated osteogenic differentiation of BMSCs. Journal of cellular and molecular medicine 2018; 22:2162-76.
31. Zheng Y, Devitt C, Liu J, Mei J, Skapek S X. A distant, cis-acting enhancer drives induction of Arf by TgfP in the developing eye. Developmental biology 2013; 380:49-57.
32. Lian J, Wu X, Liu Y, Qiu W, Zhu X, Wang X, et al. Potential roles of miR-335-5p on pathogenesis of experimental periodontitis. Journal of periodontal research 2020; 55:191-8.
33. Qiu W, Wu H, Hu Z, Wu X, Tu M, Fang F, et al. Identification and characterization of a novel adiponectin receptor agonist adipo anti-inflammation agonist and its anti-inflammatory effects in vitro and in vivo. Br J Pharmacol 2021; 178:280-97.
34. Bochenek G, Hasler R, El Mokhtari N E, Konig I R, Loos B G, Jepsen S, et al. The large non-coding RNA ANRIL, which is associated with atherosclerosis, periodontitis and several forms of cancer, regulates ADIPOR1, VAMP3 and C11ORF10. Human molecular genetics 2013; 22:4516-27.
35. Kim J H, Kim K, Kim I, Seong S, Lee K B, Kim N. BCAP promotes osteoclast differentiation through regulation of the p38-dependent CREB signaling pathway. Bone 2018; 107:188-95.
36. Koga Y, Tsurumaki H, Aoki-Saito H, Sato M, Yatomi M, Takehara K, et al. Roles of Cyclic AMP Response Element Binding Activation in the ERK1/2 and p38 MAPK Signalling Pathway in Central Nervous System, Cardiovascular System, Osteoclast Differentiation and Mucin and Cytokine Production. International journal of molecular sciences 2019; 20.
37. Lee K, Seo I, Choi M H, Jeong D. Roles of Mitogen-Activated Protein Kinases in Osteoclast Biology. International journal of molecular sciences 2018; 19.
38. Tao X, Qi Y, Xu L, Yin L, Han X, Xu Y, et al. Dioscin reduces ovariectomy-induced bone loss by enhancing osteoblastogenesis and inhibiting osteoclastogenesis. Pharmacological research 2016; 108:90-101.
39. Wu X, Qiao S, Wang W, Zhang Y, Shi J, Zhang X, et al. Melatonin prevents peri-implantitis via suppression of TLR4/NF-κB. Acta biomaterialia 2021; 134:325-36.
40. Karreth F A, Tay Y, Perna D, Ala U, Tan S M, Rust A G, et al. In vivo identification of tumor-suppressive PTEN ceRNAs in an oncogenic BRAF-induced mouse model of melanoma. Cell 2011; 147:382-95.
41. Yu L, Tu Q, Han Q, Zhang L, Sui L, Zheng L, et al. Adiponectin regulates bone marrow mesenchymal stem cell niche through a unique signal transduction pathway: an approach for treating bone disease in diabetes. Stem cells (Dayton, Ohio) 2015; 33:240-52.
42. Wang Y, Du Y, Yuan H, Pan Y, Wu J, Du X, et al. Human amnion-derived mesenchymal stem cells enhance the osteogenic differentiation of human adipose-derived stem cells by promoting adiponectin excretion via the APPL1-ERK1/2 signaling pathway. IUBMB life 2020; 72:296-304.
43. Wu X, Qiu W, Hu Z, Lian J, Liu Y, Zhu X, et al. An Adiponectin Receptor Agonist Reduces Type 2 Diabetic Periodontitis. J Dent Res 2019; 98:313-21.
44. Yang S, Liu H, Liu Y, Liu L, Zhang W, Luo E. Effect of adiponectin secreted from adipose-derived stem cells on bone-fat balance and bone defect healing. J Tissue Eng Regen Med 2019; 13:2055-66.
45. Salmi A, Quacquarelli F, Chauveau C, Clabaut A, Broux O. An integrative bioinformatics approach to decipher adipocyte-induced transdifferentiation of osteoblast. Genomics 2022; 114:110422.
46. Zhang L, Fu X, Ni L, Liu C, Zheng Y, You H, et al. Hedgehog Signaling Controls Bone Homeostasis by Regulating Osteogenic/Adipogenic Fate of Skeletal Stem/Progenitor Cells in Mice. Journal of bone and mineral research: the official journal of the American Society for Bone and Mineral Research 2022; 37:559-76.

### Example 2

### Introduction

A recent report from the Centers for Disease Control and Prevention (CDC) shows that 47.2% of American adults over age 30 have periodontal disease (PD) and the incidence of PD rises to 70.1% in adults 65 years and older (1). Based on the high prevalence, PD is considered a common public health problem. The basic pathology of PD is the immuno-inflammatory host response against commensal bacteria and excessive alveolar bone resorption leading to tooth loss (2). Furthermore, PD can trigger general systemic inflammation, adversely influencing cardiovascular, neural, reproductive, and endocrine systems (3-5). As a complex chronic inflammatory and immune disease, the molecular mechanisms of PD are not fully understood yet. The primary goal of periodontitis treatment is to prevent the progression of the disease, manage its symptoms, and restore the health of the gingiva and supporting structure of the teeth. Periodontitis treatment typically involves both non-surgical and surgical therapies (6). A pressing need exists for an innovative treatment that blocks the major elements of host mediated pathogenesis and stimulates endogenous regeneration.

Noncoding RNAs (ncRNAs) are a large segment (more than 80%) of the transcriptome that lack apparent protein encoding capability but are functionally important. Long noncoding RNAs (lncRNAs) are a subgroup of ncRNAs with a length of more than 200 nucleotides. Emerging evidence suggests that lncRNAs participate in a wide repertoire of genome organization and life processes such as growth and development, cell proliferation, differentiation, immune response, and certain diseases, such as cancer and cardiovascular diseases (7,8).

LncRNA ANRIL (referred to herein as ANRIL), situated on chromosome 9p21.3 in humans, is a lncRNA encoded in the opposite direction within the INK4/ARF locus. ANRIL is the antisense lncRNA of cyclin-dependent kinase inhibitor 2A (CDKN2A) and CDKN2B, both of which are protein coding genes situated in the INK4 locus. ANRIL has been proven to be a shared risk factor for atherosclerosis, periodontitis, diabetes, and cancer (9-11). ANRIL is the best replicated genetic locus of atherosclerosis-associated coronary artery disease (CAD) and PD (12,13). The core risk haplotype shared between CAD and PD is located at the 3′ end of ANRIL, which implies ANRIL is a prime functional candidate involved in the risk mediating mechanism (14). It can modulate genes and pathways related to inflammation, cell cycle regulation, and atherosclerosis. Additionally, studies suggest a bi-directional association between periodontitis, diabetes, and CAD (5, 9, 15). For example, Individuals with diabetes have an increased likelihood of developing periodontitis and those with both periodontitis and diabetes tend to exhibit poorer glycemic control. Thus, there are strong correlations among the three chronic inflammatory diseases: atherosclerosis, PD, and diabetes. ANRIL, as a shared risk factor in these conditions, likely plays a pivotal role in their crosstalk, specifically in how these diseases mutually influence each other through the regulatory effect of ANRIL. The PD-related ANRIL studies have reported that ANRIL is remarkably lower in the peripheral blood of PD patients in Iran (16) and significantly under-expressed in the inflamed gingival tissue in European populations (17). Downregulated ANRIL inhibits the osteogenic differentiation of periodontal ligament cells (PDLCs) (18). In addition, it was also shown that elevated highly sensitive C-reactive protein (hsCRP), a marker for systemic inflammation and a risk marker for periodontitis, was significantly associated with ANRIL SNP rs1333048 (19). These studies suggest the potential of lncRNAs as diagnostic biomarkers and targets for the treatment of PD.

We know the disruption of the homeostatic balance of bone remodeling and inflammation both play crucial roles in PD development. However, how ANRIL regulates bone regeneration and inflammation remains unclear. Therefore, our study intentionally focused on the AK148321 region (i.e., mouse Gm12610), an orthologous counterpart to human ANRIL on mouse chromosome 4, to delve into ANRIL's regulatory role and therapeutic potential in the context of PD using a mouse model. We deliberately selected this region based on its conserved genomic homology with the human ANRIL locus, ensuring meaningful translatability to the human condition. Additionally, this region's relevance to PD and the capacity to leverage a genetically modified animal model (i.e., the chr4 Δ70 kb/Δ70 kb mouse model), allowed for a thorough investigation into ANRIL's functional implications. By utilizing this region, we aimed to assess the therapeutic impact of ANRIL modulation, providing crucial insights into potential therapeutic strategies that target PD. We coined the term lncRNA-APDC (APDC) for the AK148321 region, reflecting its major functions in atherosclerosis, periodontitis, diabetes, and cancer. APDC maintained the synteny of ANRIL with its neighboring genes Cdkn2a, Cdkn2b, and methyl-thioadenosine phosphorylase (Mtap) (20), and preserved its functions as mitigating neuroinflammation and atherosclerosis (21,22). Inflammation and uncoupling of bone formation and resorption were studied in both APDC deficient and wild-type mice with and without periodontitis. The objective of this study was to discover the preventive and/or therapeutic impact of APDC in periodontitis and elucidate the underlying molecular mechanisms of action.

### Materials and Methods

### The Experimental Periodontitis Model

The 3D printing technology was applied in our modified experimental periodontitis surgery, which can dramatically reduce trauma and bleeding and shorten the duration of the procedure. The mice were anesthetized with ketamine (100 mg/kg) and xylazine (10 mg/kg) through intraperitoneal (IP) injection. Ligatures were held by the 3D-printed handle and locked tightly. We used a dental explorer to create a tiny gap between the molars and guide the suture. Ligatures were placed around the second molar and tied on the buccal side. After the EP surgery, the inserted ligatures were checked every 3 days and kept in place. All the animals that entered the study survived until the time point for sacrificing and sample collection. The periodontal tissue, alveolar bone and blood were collected. The 3D printed surgical tools were originally developed by Dr. Jiao's lab at the University of North Carolina at Chapel Hill (70). We modified the structure of the suture lock to extend its usage with the ability to apply ligature surrounding second molar. The tools were 3D printed with Tough 2000 Resin (Formlabs, Boston, MA) at the Hubs platform. All animals were housed at Tufts Comparative Medicine Services (CMS), with food and water provided ad libitum. A 12-hour light and 12-hour dark cycle was maintained during the experimental period.

### APDC Knockout (KO) Mouse Model and Genotyping

The APDC KO mouse model (129S6/SvEvTac-Del(4C4-C5)1Lap/Mmucd, MMRRC_032091-UCD) was obtained from the Mutant Mouse Resource & Research Center (MMRRC). In creating this model, lncRNA APDC (also known as Gm12610, AK148321) was deleted in W4/129S6 ES cells through Cre/loxP recombination. APDC was confirmed to be severely reduced in these chr4 Δ70 kb/70 kb mice (20). To further confirm the APDC-null genotypes of this strain, all APDC-KO and control mice enrolled in this study underwent confirmational genotyping. Tail DNA samples were extracted by DNeasy Blood & Tissue Kits, and genomic DNA genotyping analysis (QIAGEN, MD, USA) was performed with standard gDNA qPCR and gel electrophoresis. All animals with the same genotype same gender were randomized to the groups.

### Microcomputed Tomography (μCT) Analysis

The maxilla of the mice from the experimental and control group were scanned using an animal micro-computed tomography (CT) system (SkyScan1172; Bruker-microCT, Belgium) at Tufts Medical Center. The 3D models were then reconstructed from the raw images with Bruker NRecon and analyzed with software from Bruker, including CT Analyser 1.17.7.2, DataViewer 1.5.4.0 and CT Vox 3.3.0. The alveolar bone loss was described as the distance from the cementoenamel junction to the alveolar bone crest (CEJ-ABC), and the values were measured at six periodontal sites (mesiobuccal, midbuccal, distobuccal, mesiopalatal, midpalatal, and distopalatal) of the second molars.

### Hemotoxylin and Eosin (H&E) Staining and TRAP Staining of Bone Tissue

The left proximal tibia of APDC KO and control mice were fixed in 4% paraformaldehyde, embedded with paraffin, and cut into 5 μm sections. H&E staining and TRAP staining were performed according to the manufacturer's instructions. Digital images were taken with an OLYMPUS BX53 microscope (Olympus, Waltham, MA).

### Immunoassay

The serum samples were evaluated with the V-PLEX Plus Proinflammatory Panel1 Mouse Kit on the Meso Scale Discovery (MSD) MESO QuickPlex SQ 120 (MESO SCALE DIAGNOSTICS, Rockville, MD). The pro-inflammation cytokines: IFN-γ, IL-10, IL-2, IL-4, IL-5, IL-6, KC/GRO, IL-10, IL-12p70, and TNF-α were quantitative determined.

### Cell Culture

Bone marrow-derived mesenchymal cells (BMSCs) were harvested from six-week-old APDC KO and wild type mice. The mice were first killed by cervical dislocation following anesthesia respectively, and then sterilized with 70% (vol/vol) ethanol. The hind legs were removed from the body using scissors and all skin and muscle tissues were removed from the legs. The epiphyses of femur and tibia were cut off. The bone marrow was flushed out with Dulbecco's modified Eagle's medium (DMEM; Gibco® by Life Technologies™) from the long bones, then cultured in the DMEM with 10% fetal bovine serum (FBS) and 1% penicillin/streptomycin in a 5% CO2 incubator, at 37° C. After 24 hours, nonadherent cells were carefully removed and fresh medium was added. The BMSCs were treated with osteoblast differentiation medium (DM) including ascorbic acid (Asc, 50 g/ml), dexamethasone (Dex, 0.1 μM) and 0-glycerophosphate (β-Gly, 10 mM) for 3 days and differentiated to the osteoblasts (OBs).

### Next Generation Bulk RNA Sequencing

The RNA library was prepared by Illumina Ribo-Zero Plus rRNA Depletion Kit followed by NEBNext® Ultra™ II RNA Library Prep Kit for Illumina®. The sequencing was performed on Illumina Novaseq 6000 S4 Flowcell (PE150). The minimum coverage per sample is 40M Read Pairs (12G). All samples passed the quality control (QC).

RNA Isolation and Real-Time Polymerase Chain Reaction (RT-PCR) The total RNA of tissues and cells was isolated using TRIzol (Thermo Fisher Scientific) and the Quick-RNA™ MiniPrep Kit (Zymo, CA). RNA of each sample was reverse-transcribed by M-MLV Reverse Transcriptase (Promega, WI) and cDNAs were quantified by RT-PCR with SYBR Green Supermix (Affymetrix) on Bio-Rad iQ5 (Bio-Rad Laboratories, CA).

### Preparation of Single-Cell Suspensions

The gingival tissue surrounding the second molar was harvested after the ligature placement for 10 days from lncR-APDC knockout mice and control mice (n=4 per group). The gingival tissues were minced into small fragments (less than 1 mm3) by a #10 surgical blade. The minced sample was then transferred to a gentleMACS C tube (130-093-237, Miltenyi Biotech) with 10 ml tissue culture media (RPMI with 2% FBS). C tubes were attached to the gentleMACS dissociator (130-093-235, Miltenyi Biotech) upside down, then performed program “m_lung_01.02”. Centrifuge C tube at 300 g for 5 mins. The supernatant was disposed and 10 mL of digestion solution (2 mg/ml type II collagenase, 2 mg/ml Dispase II, 0.04 mg/ml DNase in RPMI media) was added. The C tubes were incubated for 45 mins in a 37° C. shaker. C tubes were attached to gentleMACS dissociator again and ran program “m_lung_02.01”. Samples were then centrifuged, resuspended with 0.5 ml tissue culture media, and filtered with 70 μm cell strainers. Fluorescence-activated Cell Sorting (FACS) was performed to remove the dead cells (3 μM DAPI) and increase the viability above 90%. After cell counting, samples with the final concentration of 700-1200 cells/μl were stored on ice until further processing. The whole procedure was performed on ice whenever possible.

### Single-Cell RNA Sequencing and Data Processing

Barcode specifically labeled the transcripts of each signal cell by using a Chromium Next GEM Single Cell 3′ Kit v3.1 (10× Genomics Inc, San Francisco, USA). Libraries were constructed and sequenced on an Illumina NovaSeq 6000 platform (S100D032203) at a sequencing depth of ˜800 million reads in Tufts University Core Facility (TUCF). The sequencing results were demultiplexed and cell barcodes were extracted. Subsequently, Single-Cell Analysis in Python (SCANPY) (26) was utilized for data clustering analysis and visualization. SCSA (29), an automatic tool and distinct marker genes from published papers was used for cluster annotation. The ligand-receptor interaction data were generated by Squidpy (58) (based on CellPhoneDB (59) and Omnipath (60) database) via screening and paring the ligands and receptors, which expressed in more than 10% of cells in each cluster. The lncRNA-RNA reaction tool, intaRNA (52,71), was used to predict the direct bind site of IncR-APDC and Tff2. The newest intaRNA package was downloaded from GitHub to perform the analysis, which was able to analyze more than 2000 nt full length of lncRNAs and can be optimized on multiple settings.

### AAV Delivery System

We designed the AAV9-CAG-APDC (Charles River Laboratories) for the delivery of APDC (SEQ ID NO: 33) in vivo. The AAV9 with CAG promoter-driven expression of RFP (Charles River Laboratories, CV17176-AV9) was utilized to visualize the APDC delivery. The AAV9-CAG-APDC and control AAV vector were injected after 3 weeks of EP surgery. The animals were sacrificed after another 3 weeks.

### Statistics

All data are presented as means±SD of results obtained from three or more experiments. The significance of differences in various categorical variables was evaluated using one-way analysis of variance for multigroup comparisons and the t test for between two group comparisons. A p value of less than 0.05 was considered to be statistically significant. All statistical analyses were performed using SPSS statistics v27 (SPSS Inc., IL) and Prism GraphPad 9.0 (GraphPad Software).

### Results

### APDC Deficiency Aggravates Bone Loss and Inflammation Caused by Periodontitis

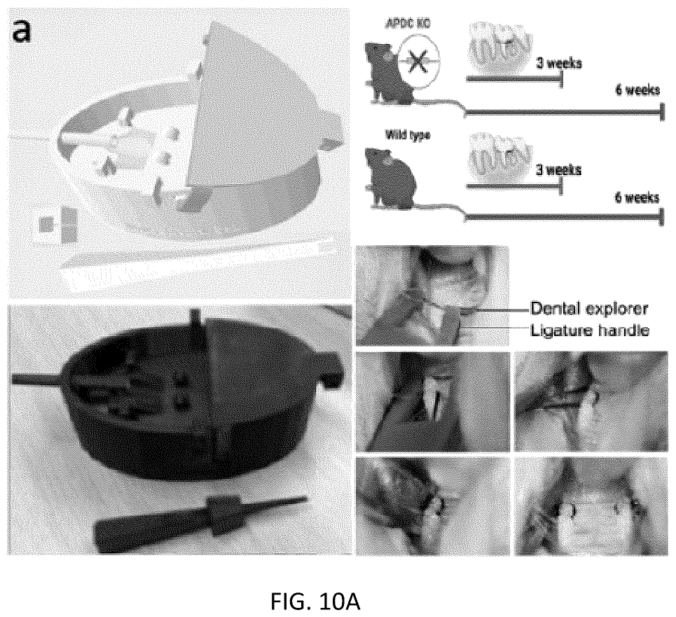

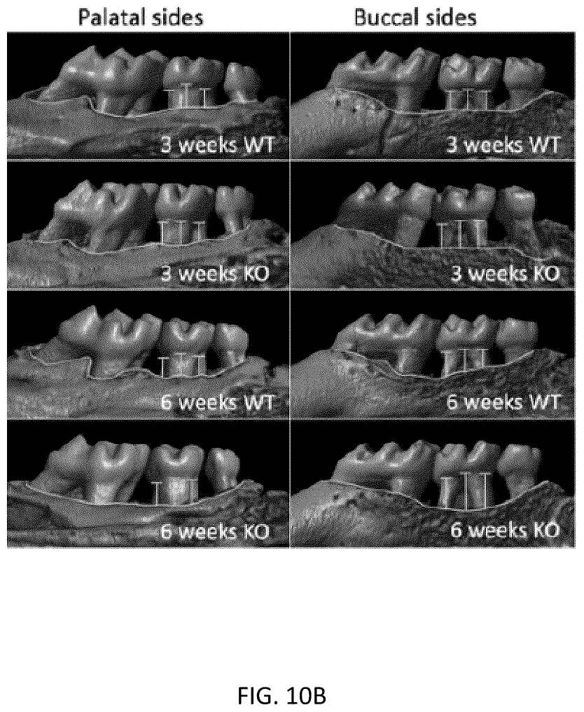

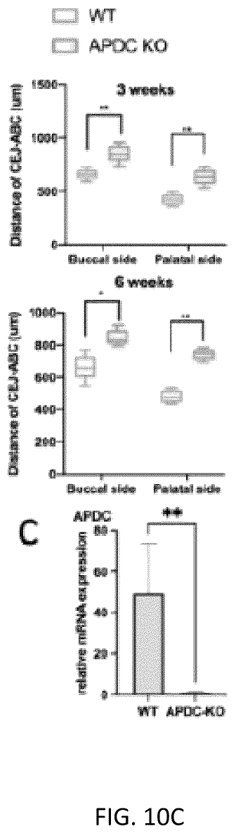

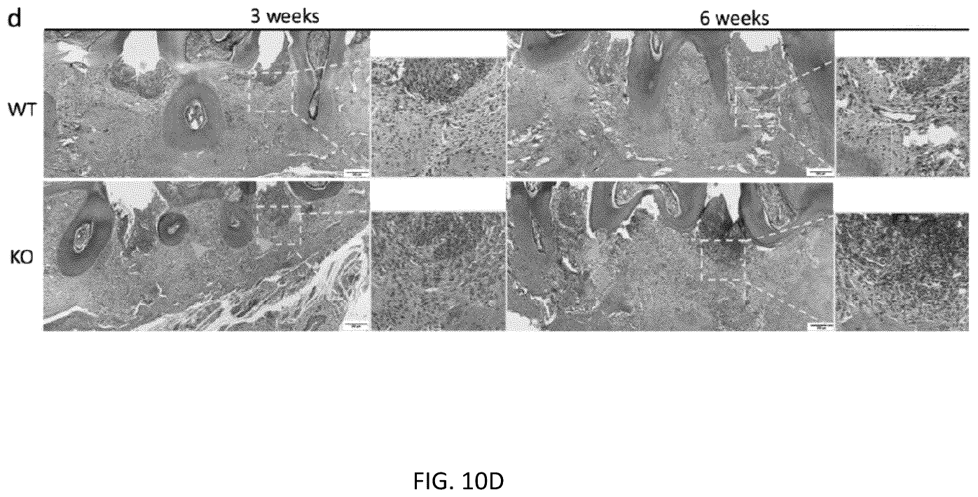

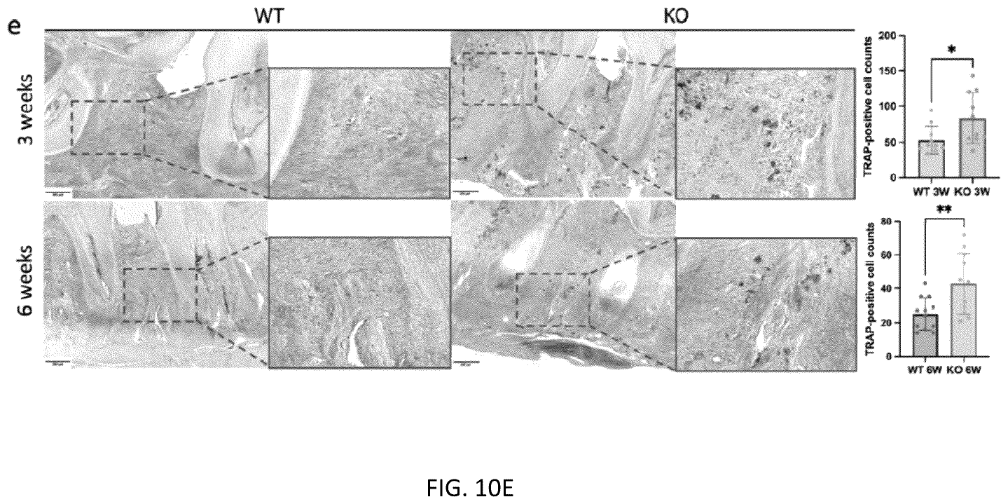

The experimental periodontitis (EP) model was performed on 4-month-old female APDC-KO and wild-type mice with 3D printed surgical tools (FIG. 10A). Ligatures were placed surrounding the second molar on both sides of the maxilla for 3 weeks and 6 weeks (n=8-10 per group). Micro-CT reconstruction showed that the distance from the cementoenamel junction to the alveolar bone crest (CEJ-ABC) was significantly increased in the APDC-KO groups (1) by 29% on the buccal side and 49% on the palatal side at 3 weeks, and (2) by 27% on the buccal side and 54% on the palatal side at 6 weeks compared to the control groups at the same time points (FIG. 10B). These changes indicate more severe alveolar bone resorption in the KO group during different stages of periodontitis development. The successful deletion of APDC was confirmed by the expression level of APDC in KO and wildtype mice (FIG. 10C). H&E histology staining demonstrated intense inflammatory cell infiltrate in the tissue subjacent to the periodontal pocket, exacerbated bone resorption of the alveolar crest between the molars and cementum resorption (i.e., discontinuous cementum) in the APDC-KO group at both 3 and 6 weeks (FIG. 10D). The number of tartrate resistant acid phosphatase (TRAP) positive cells on the alveolar bone surface remarkably increased in the APDC-KO groups compared to the control group at the same time points (FIG. 10E). Taken together, the findings reveal the impact of APDC deficiency on alveolar bone loss and inflammatory infiltrate in periodontal tissues.

### APDC Plays a Regulatory Role in Bone Metabolism

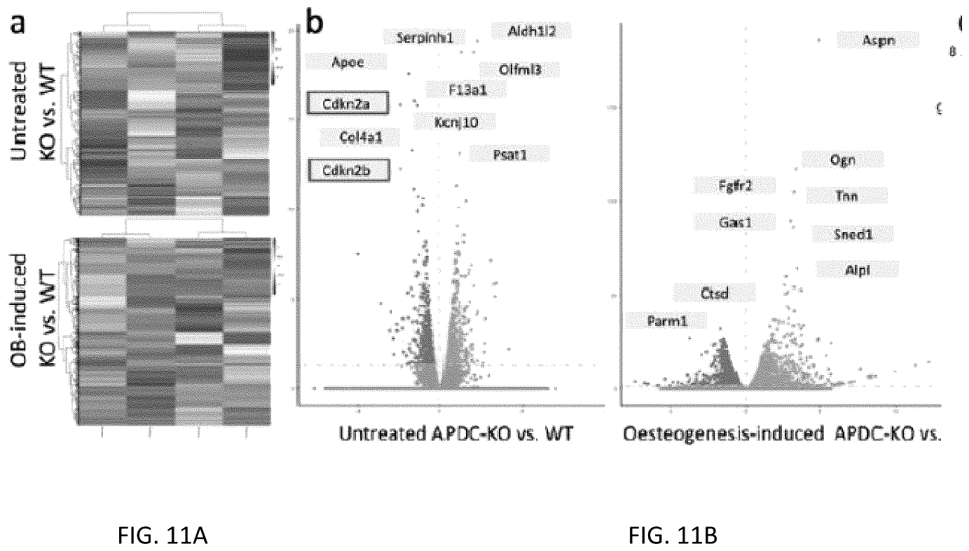

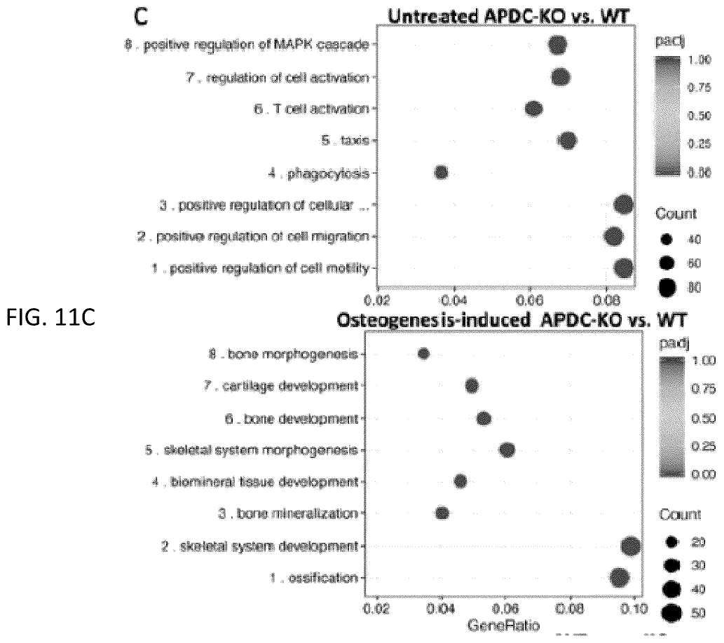

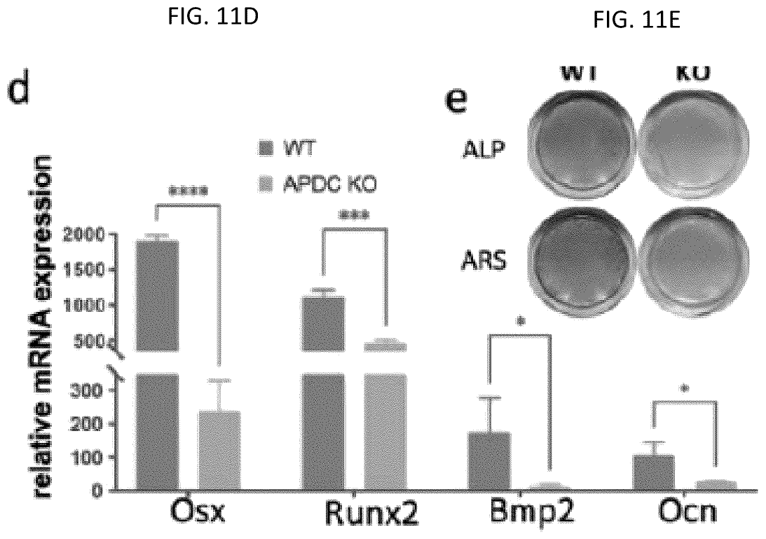

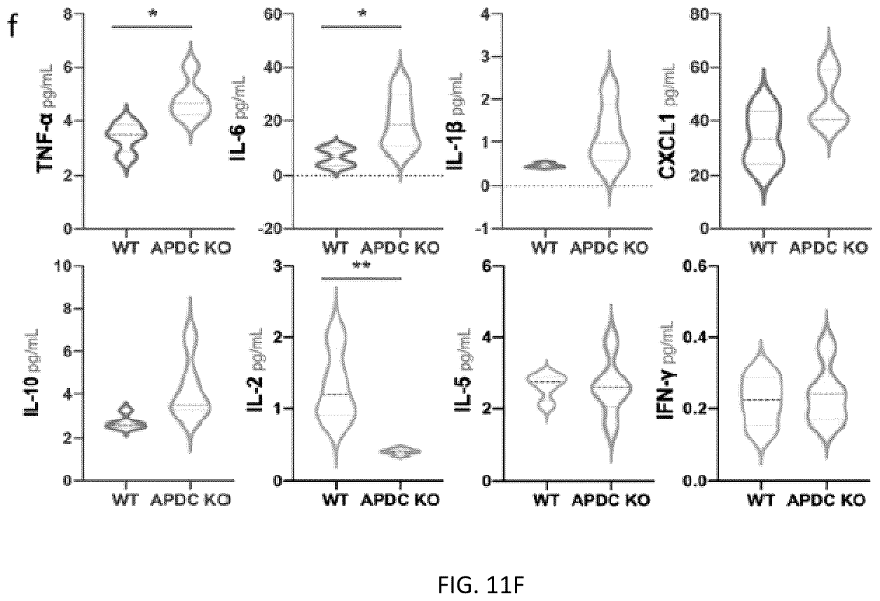

To determine the direct impact of APDC on the homeostatic balance of bone remodeling, we performed bulk RNA sequencing on untreated and osteogenic differentiated bone marrow mesenchymal stem cells (BMSCs). The overall gene profiling of untreated and osteogenic differentiated APDC-KO and wildtype BMSCs are presented in a heatmap (FIG. 11A). The top differentially expressed genes (DEG) are marked on the volcano plots (FIG. 11B). Interestingly, Cdkn2a and Cdkn2b are among the top DEGs in the untreated BMSCs. These results are the first data obtained regarding Cdkn2a and Cdkn2b expression levels in BMSCs of APDC knockout mice, and they are consistent with the observations by other researchers on other tissue types (20,22). The GO (gene ontology) analysis of untreated KO, compared with wild-type BMSCs, shows that downregulated DEGs primarily pertain to the following biological processes: cell motility and migration, T lymphocytes (T cell) and leukocyte activation, as well as differentiation and activation of the immune response. Conversely, in osteogenic-differentiated groups, APDC deficiency impacts the gene functions of ossification, skeletal system development and morphogenesis, bone mineralization and osteoblast differentiation (FIG. 11C Furthermore, our in vitro BMSCs differentiation showed that the osteogenic markers, including Osx, Runx2, Bmp2 and Ocn, are remarkably downregulated in the APDC-KO group (FIG. 11D). ALP staining, which marks hypertrophic cells before mineralization onset, and ARS staining, which shows calcium-rich deposits in cells, were both decreased in the APDC-KO group (FIG. 11E). These results suggest that APDC deficiency disturbs immune response and bone metabolism.

### APDC Silencing Dysregulates the Expression of Pro-Inflammatory and Anti-Inflammatory Cytokines in the Host Immune Response of Periodontitis

A wide range of pro-inflammatory and anti-inflammatory cytokines produced by immune cells play a vital role in initiating and regulating the inflammatory process of periodontitis (23,24). The activation of the acquired immune response by a periodontal pathogen can interfere with bone coupling by stimulating osteoclastogenesis and limiting the formation of new bone following resorption (25), ultimately resulting in alveolar bone loss. We utilized an immunoassay to determine the cytokines involved in the innate and adaptive immune responses of periodontitis. The pro-inflammatory cytokines TNF-α and IL-6 were increased in the APDC-KO mice after 3 weeks of periodontitis induction, whereas the pleiotropic cytokine IL-2, which mediates both pro- and anti-inflammatory functions, was significantly decreased (FIG. 11F).

### Performing Single-Cell RNA Sequencing to Identify Cell Types in the Gingival Samples

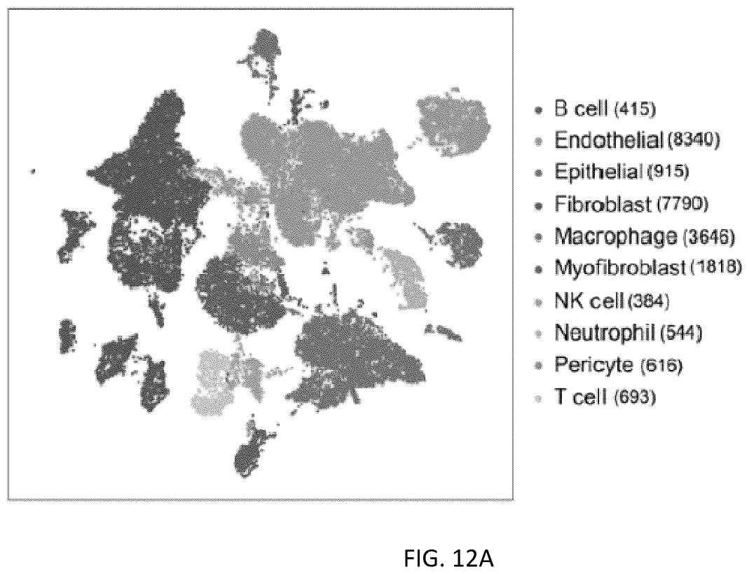

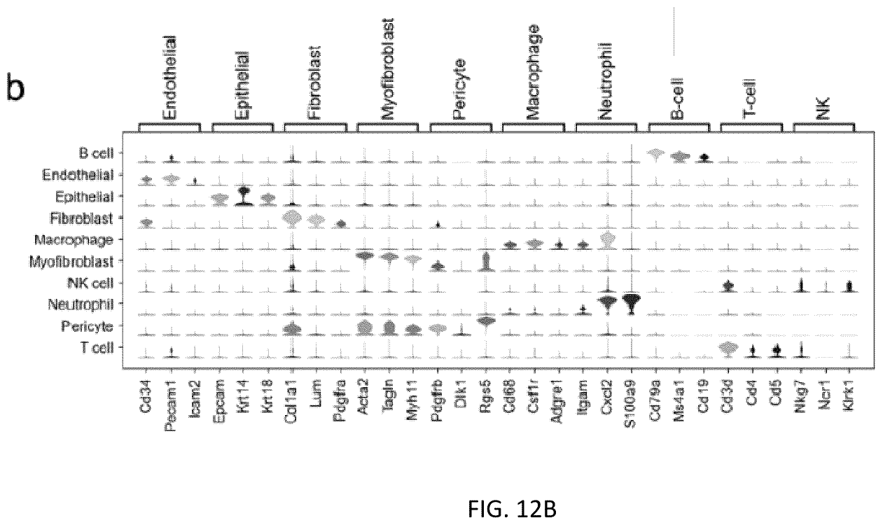

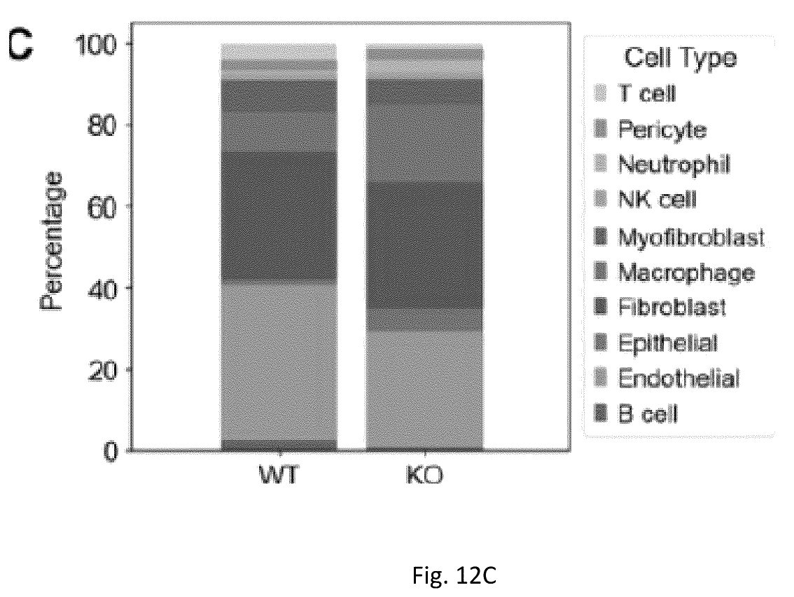

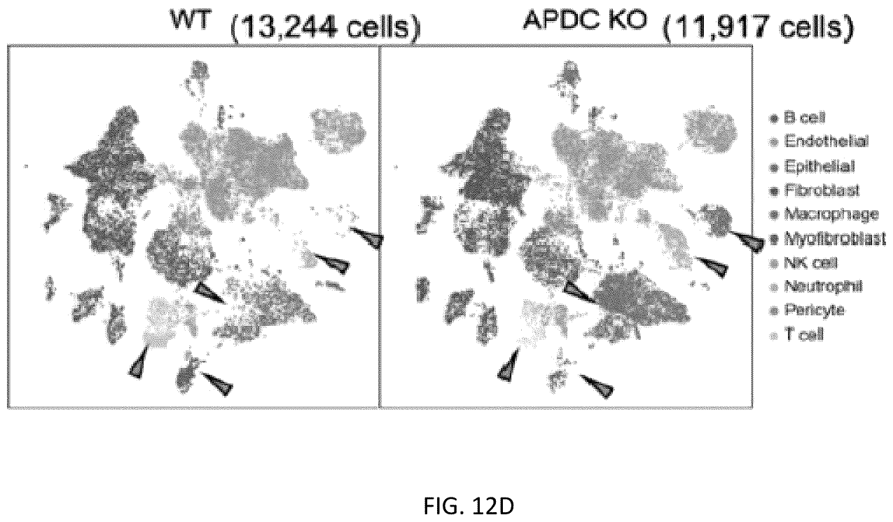

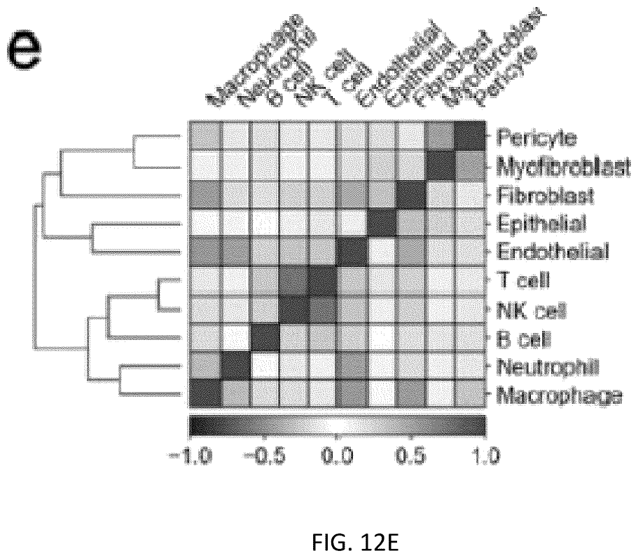

To uncover the mechanism of APDC's impact on periodontitis at single-cell resolution, we collected gingival tissue surrounding upper-second molars from 4-month-old female APDC-KO and wild-type mice 10 days after ligature placement. The samples underwent dissociation, yielding single cells with a viability >90% which were then processed using barcoding, reverse transcription, cDNA amplification, library construction, and sequencing. The raw sequencing data was aggregated in Cell Ranger. Data analysis and visualization were performed in SCANPY (26) on 25,161 single-cell transcriptomes after data quality filtering. We performed unbiased clustering of single cells using marker genes from the published literature (27,28) and SCSA, a tool based on a score annotation model (29). The resulting cells were annotated into 10 clusters (FIG. 12A). The marker genes for each cluster and their expression scores are presented in stacked violin plots (FIG. 12B). The correlations of annotated cell types are presented in a correlation matrix with a dendrogram (FIG. 12E). This matrix is the mathematical representation that reflects the biological correlations between cell types. The consistency of the correlation pattern and the known features of different cell types (e.g., gene profiles, functions, and shared precursors) confirmed the accuracy of the annotation results. For example, in our samples, the annotated immune cells, including T cells, NK cells, B cells, macrophages and neutrophils, have strong correlations compared to the other cell types. Intriguingly, the inflamed gingival samples of APDC-KO mice showed significant differences in the distribution and proportion of multiple cell types, in contrast to the wild-type control group, even though both groups received identical treatment. Notable alterations included a significant increase in macrophages, neutrophils, and epithelial cells, along with reduced populations of T and B lymphocytes in the APDC deficient mice (FIG. 12C, FIG. 12D).

The Imbalance within the T Cell Population is Due to the Lack of CD8+ Cytotoxic T Cells

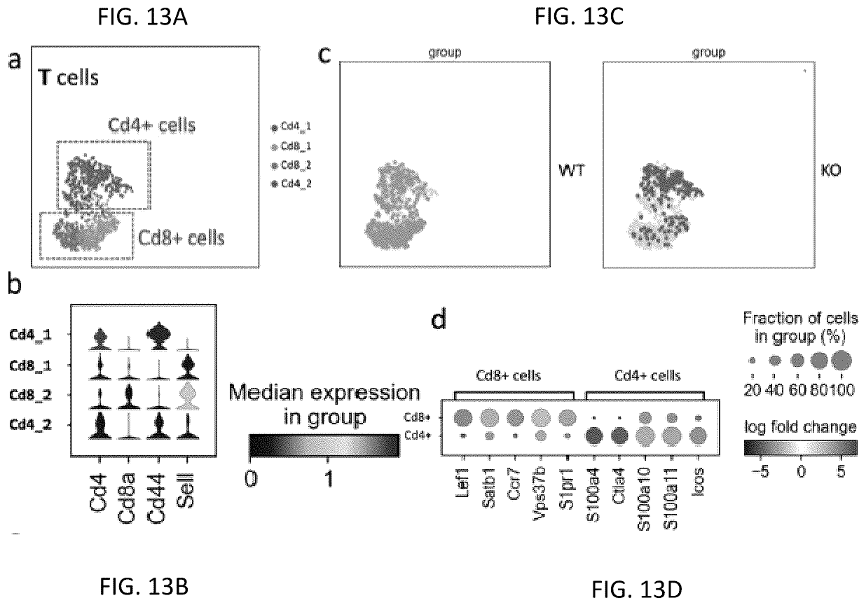

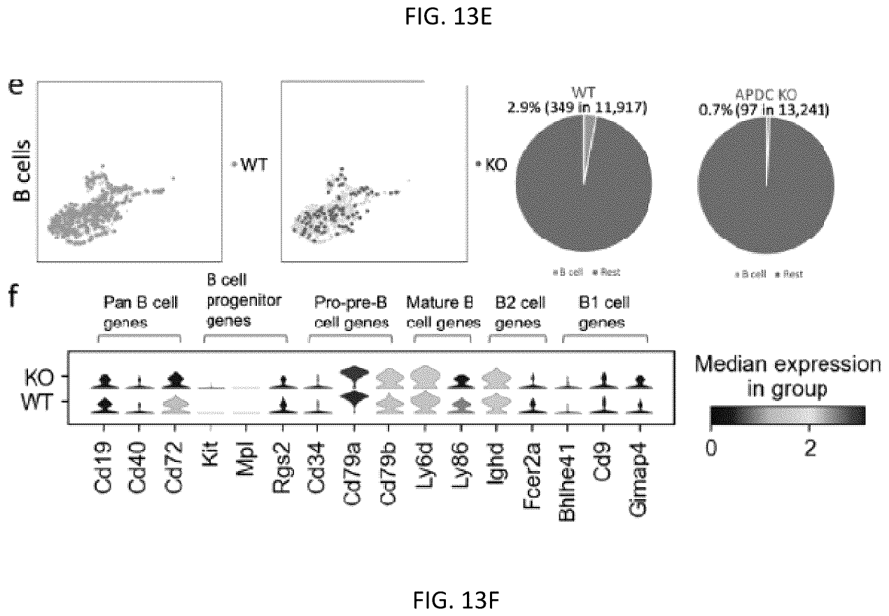

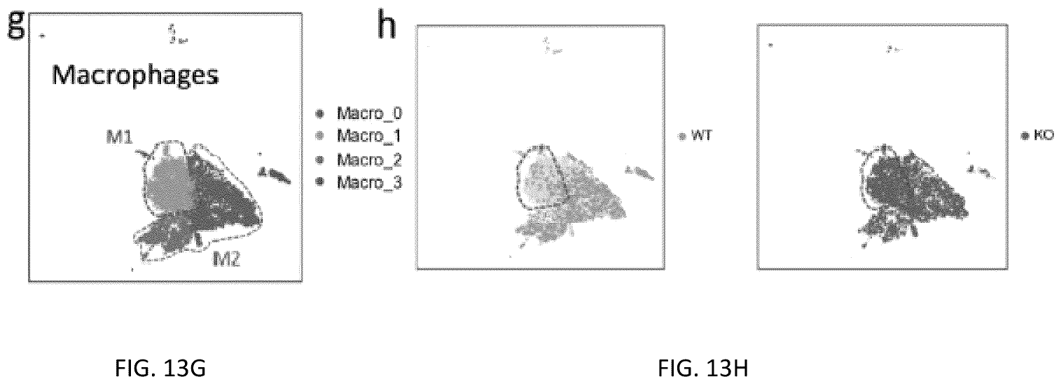

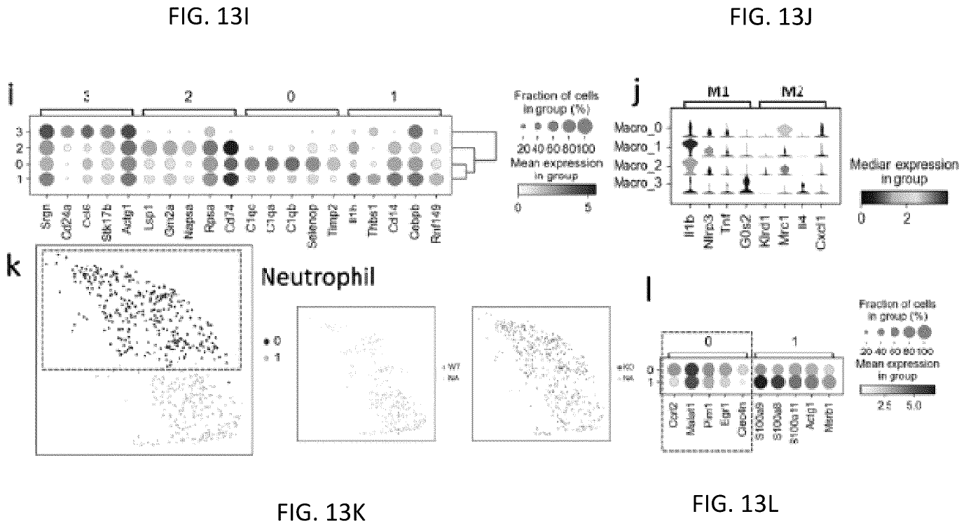

The predominant immune cell types observed in gingival tissue within 10 days after ligature placement are neutrophils, macrophages, and T cells (30). The T cell is one of the major types of immune cells involved in the host defense and control of immune-mediated periodontitis development. T cells have two main populations, CD4+ T cells (i.e., helper or inducer) and CD8+ T cells (i.e., cytotoxic or suppressor), and can be further categorized as memory or naïve based on the expression of Sell and CD44 (31). The Cd44lowSell+ population is considered naïve (TN), the Cd44highSell+ population is classified as central memory T cells (TCM) and the Cd44highSell− cells are categorized as effector and/or effector memory T cells (TE/TEM) (32). In our data, the T cell cluster was further divided into four subclusters (FIG. 13A). The gene expression level of Cd4, Cd8a, Cd44 and Sell was utilized to identify the subtypes (FIG. 13B). The T cell cluster was also represented based on different groups (i.e., APDC-KO and wild type; FIG. 13C) and DEGs between Cd4+ and Cd8+ clusters (FIG. 13D). Balancing the functionally different T cell subsets is crucial in immune regulation. Notably, the APDC-KO EP group was found to lack Cd8+ cells (see cluster CD8_1 and CD8_2 in FIG. 13B), which have the role of cytotoxicity as well as suppression of excessive immune activation and tissue repairing (33). Furthermore, Cd8+ T cells are also involved in bone healing. Studies have demonstrated that both mouse Cd8+ T cells and in vitro expanded human Cd8+ T cells can secrete Wnt10b and promote osteoblastogenesis (33). Additionally, Cd8+ T cells have been shown to confer bone protection by suppressing osteoclastogenesis (34). The reduced presence of Cd8+ T cells in the APDC-deficient group aligns with its phenotype of an exacerbated immune response and increased bone resorption.

### The APDC-KO EP Model Showed Reduced B Cell Counts

B cells constitute a fundamental element of the adaptive immune system, serving vital roles such as antibody and cytokine production, antigen presentation, and the recently discovered B-T reactions (35). Particularly in the context of PD, B cells produce antibodies directed against periodontal pathogens, effectively curbing the progression of periodontal inflammation (36). In our study, we noted a markedly diminished proportion of B cells across all cell types, with the APDC deficiency group registering a mere 0.7% (97 out of 13,241) compared to the wildtype's 2.9% (349 out of 11,917; FIG. 13E). The low B cell level in inflamed gingival tissue could suggest that the immune system might have difficulty mounting an effective response against the infection. (36). Moreover, using the marker genes of different B cell stages, we determined the proportion and functional level of B cells at various developmental phases (FIG. 13F). Specifically, highly expressed Cd79a, Cd79b, Ly6d and Ly86 indicate more pre-B cells and mature B cells in the wildtype group than in the APDC deficient group. In addition, Cd72 is considered to be a pan B cell marker broadly expressed from pre-B cells to mature B cells (37). Taken together, the B cells in inflamed periodontal tissue with APDC deficiency have lower counts and are less functional during the immune response.

### An Increased Pro-Inflammatory M1 Subtype of Macrophages Occurs in APDC-KO EP Mice

Macrophages are the main immune cell population in gingival tissue from day 5 in the EP mouse model (30). We performed sub-clustering on the macrophage population to gain higher resolution and a better understanding of the host inflammatory and immune mechanisms during PD progression. Four subtypes of macrophages were identified as Macro_0, Macro_1, Macro_2 and Macro_3 (FIG. 13G). Intriguingly, the Macro_1 subcluster was a much larger proportion of the whole macrophage cluster in the APDC-KO group compared to the wild-type (FIG. 13H). Therefore, we further investigated the highly expressed genes and DEGs of each subtype (FIG. 13I). The Macro_0 cluster highly expressed the C1qc, C1qa and C1qb genes. C1q polarizes macrophages toward an anti-inflammatory (M2-like) phenotype (38). The Macro_1 cluster highly expressed Ilb1 and the NOD-like receptor family pyrin domain containing 3 (Nlrp3), which is involved in M1 macrophage polarization (39). This subtype shows a strong pro-inflammatory phenotype as an M1 macrophage. The top genes of the Macro_2 cluster are Lsp1 and Gm2a, which are highly expressed in M2 macrophages. The expression of Tgfb1, an anti-inflammatory gene, was also higher in the Macro_2 cluster. The Macro_3 cluster represents a monocyte-macrophage cluster rather than mature macrophages, characterized by high expression of the Cebpb, Adgre1, and Cd24a genes. Among these subclusters, the APDC-KO group had a similar subcluster size for the Macro_0, 2, 3 subclusters, while a significantly larger Macro_1 subcluster was observed (M1 macrophages; FIG. 13J). The increase of M1 macrophages resulted in the total macrophage proportion of whole sample being doubled in the APDC-KO group compared to the wild-type (19.1% vs. 9.4%) as shown in FIG. 12B. Taken together, more M1 macrophages that produce pro-inflammatory cytokines were observed in the APDC-KO EP gingival tissue.

### Aggravated Neutrophil Recruitment was Evident in the APDC KO EP Model

Neutrophils are produced and stored in bone marrow, released into the circulation, and continuously recruited to the site of chronic inflammation (40). In PD, neutrophils are significantly enriched and play critical roles in periodontal homeostasis, including phagocytosis, degranulation, cytokine production, and neutrophil extracellular trap (NET) formation (41,42). In our study, we observed a subcluster of neutrophils, with top DEGs of Ccrl2, Egr1 and Clec4n, that only appeared in the APDC-KO group (FIG. 13K, FIG. 13L). Ccrl2 is highly expressed in primary neutrophils and plays an important role in neutrophil recruitment (43). Therefore, the higher Ccrl2+ neutrophil level indicates provoked and continuing neutrophil recruitment in the APDC-KO EP gingival samples, which is also consistent with the increased counts of total neutrophils in the APDC-KO group compared to the wild-type (3.2% vs. 1%).

Endothelial Cells, Fibroblasts, Myofibroblasts and Pericytes were Found in the Inflamed Condition

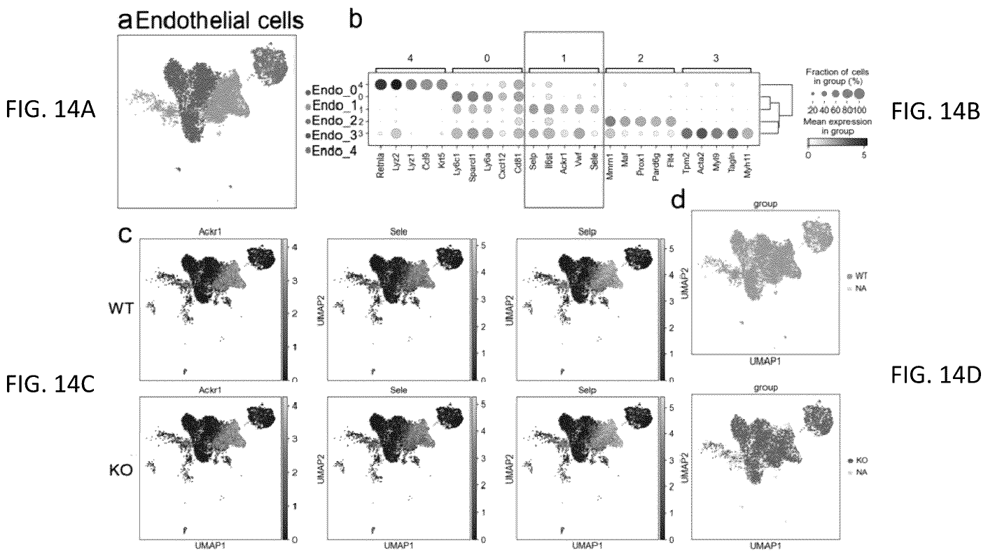

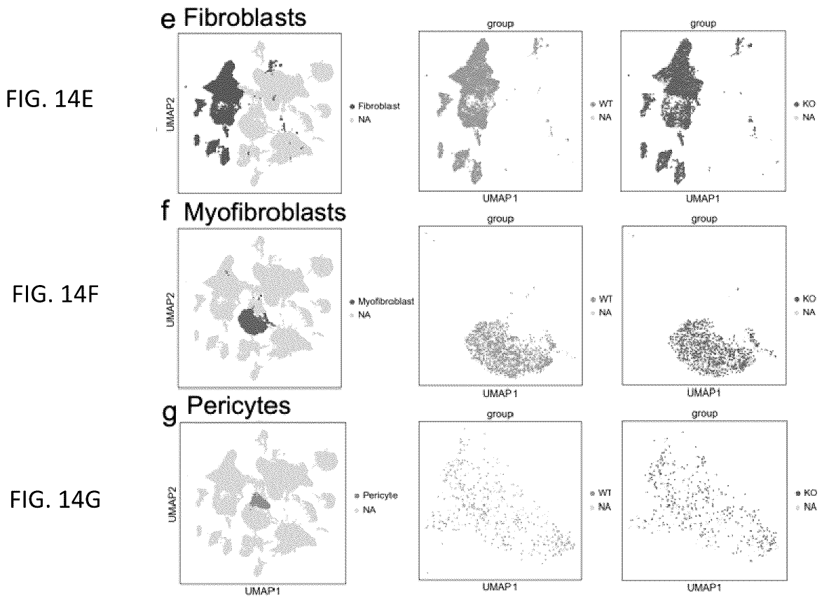

Endothelial cells were the biggest cellular component in the inflamed gingival samples. A total of five subclusters were identified (FIG. 14A). The endo_1 subcluster, characterized by high expression levels of Selp, Ackr1 and Sele (FIG. 14B), is well-documented to be closely associated with immune regulation during the PD development (44), clearly suggesting that the tissue samples are in an inflammatory condition. We further checked the related gene expression and cell counts in both the KO and wild-type groups. There was no significant disparity between the two groups (FIG. 14C, FIG. 14D).

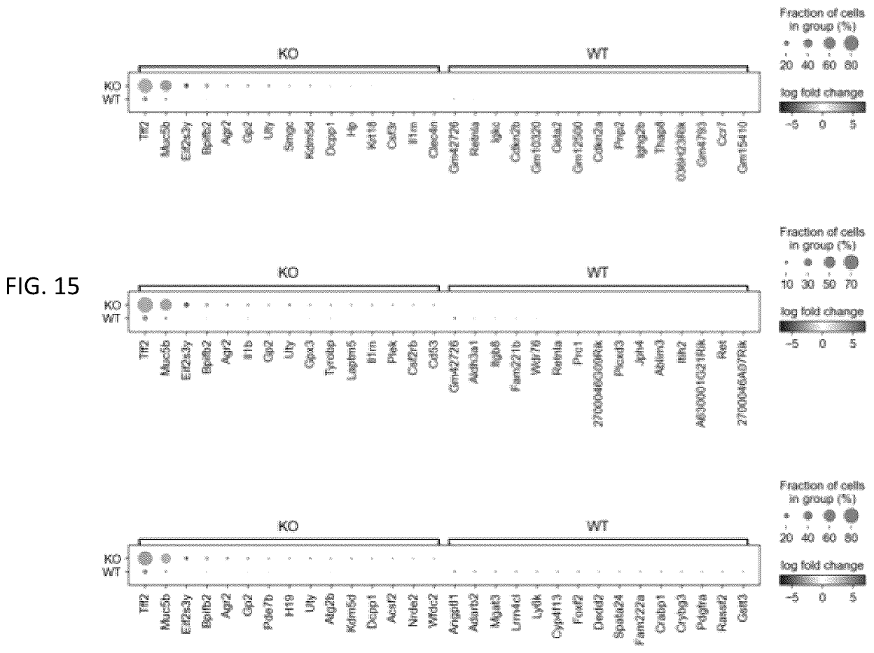

Fibroblasts are centrally involved in the wound healing response and remodeling of the periodontal tissue (45). During the remodeling phase of wound healing, a specific subtype of fibroblast known as myofibroblasts may emerge (46). Myofibroblasts may also derive from alternative sources, including mesenchymal stem cells, pericytes, and epithelial cells (47). It was recently reported that pericytes possess multilineage differentiation capacity and can be the source of tissue stem cells and/or progenitor cells, which are similar to the periodontal ligament stem cells (48). In our data, the gene profiling and cell counts for these three cell populations were consistent with other periodontitis related studies and did not show a disparity between the APDC KO group and the wild type (FIG. 14E-14G, FIG. 15).

### A Unique Subset of Epithelial Cells (Krt8+, Krt18+, Krt5−, and Krt14−) was Identified in APDC-KO Mice

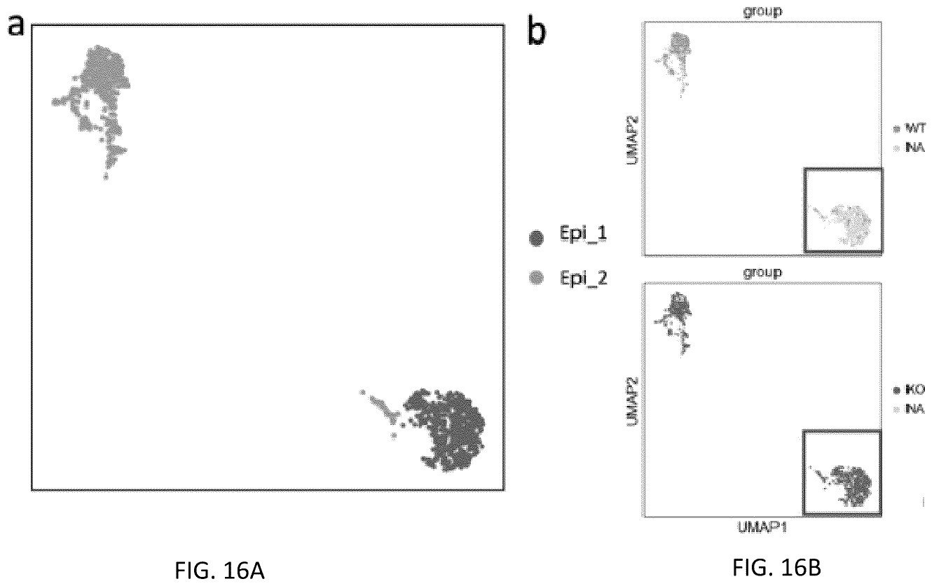

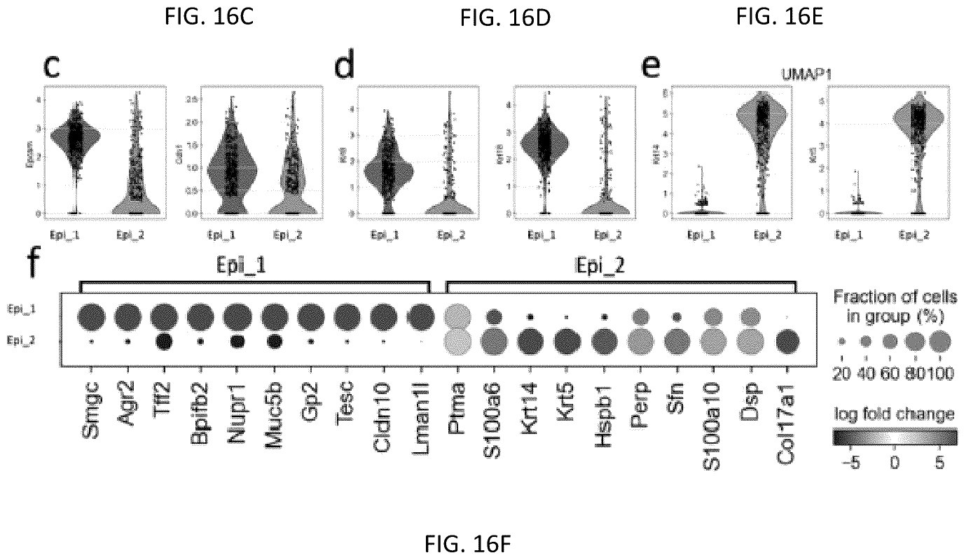

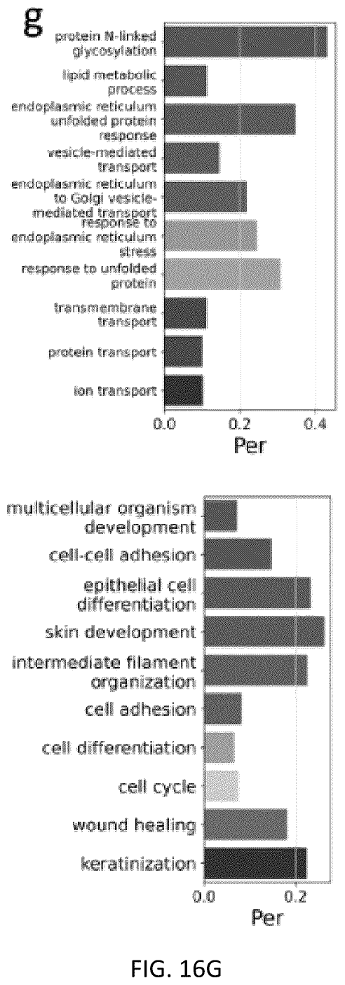

Gingival epithelial cells act as the first line of defense against periodontal pathogens providing a barrier to bacterial invasion. In this study, we identified two subsets of epithelial cells, both expressing the common epithelial marker genes: Epcam and Cdh1 (FIGS. 16A-16C). The Epi_2 cluster expressed Krt14 and Krt5 and was observed in both APDC-KO and wildtype groups (FIG. 16E). Conversely, the Epi_1 cluster represented a subset of epithelial cells expressing Krt8 and Krt18 but devoid of Krt5 and Krt14 (FIG. 16D). Krt8 is reported to be involved in the inflammatory response and usually collaborates with Krt18 to regulate protein synthesis and cell movement and inhibit apoptosis (49). Furthermore, the most highly expressed genes in the Epi_1 cluster, including Agr2, Nupr1, Muc5b and Cldn10 (FIG. 16F), have major functions as barrier maintenance and secretion of mucus and anti-microbial proteins and cytokines. GO enrichment was performed on both clusters, revealing that Epi_2 had typical epithelial-related gene functions, such as cell-cell adhesion and epithelial cell differentiation, whereas Epi_1 had distinct gene enrichment results in protein N-linked glycosylation and lipid metabolic processes. (FIG. 16G). The vast majority of this unique Epi_1 subcluster was from the APDC-KO group (FIG. 16B), suggesting that APDC deficiency alters the gene profiling of epithelial cells in PD, consequently impacting their functions.

### High Expression of Trefoil Factor 2 (Tff2) was Detected Across all Cell Types in the APDC Deficient Group

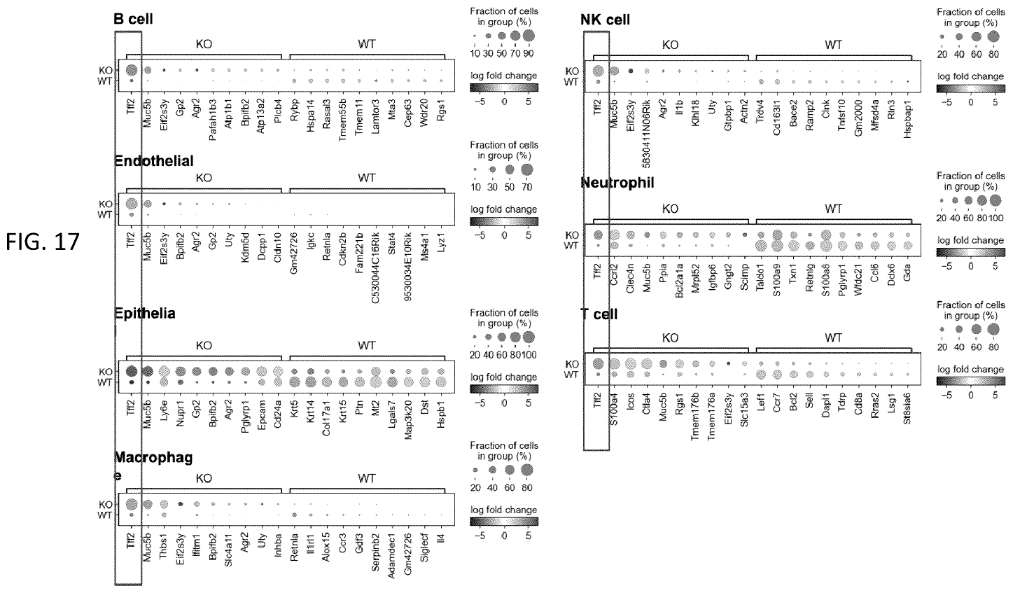

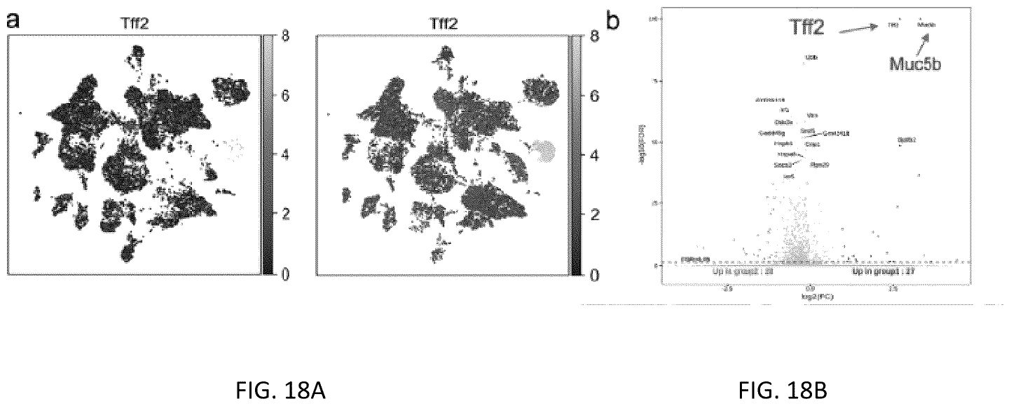

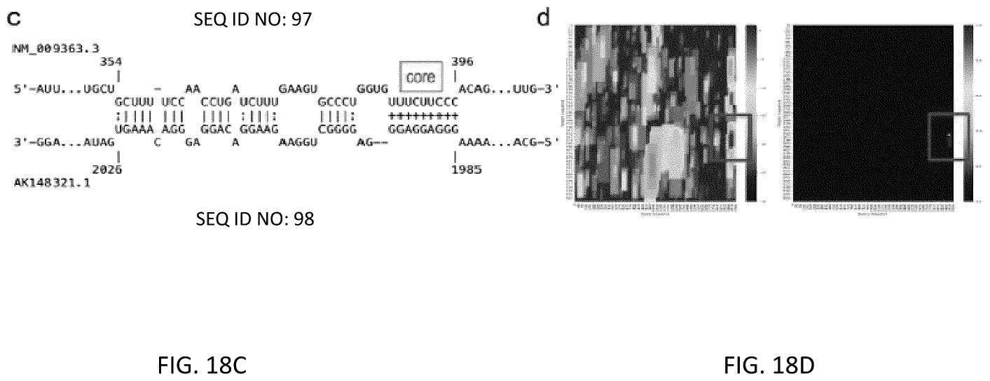

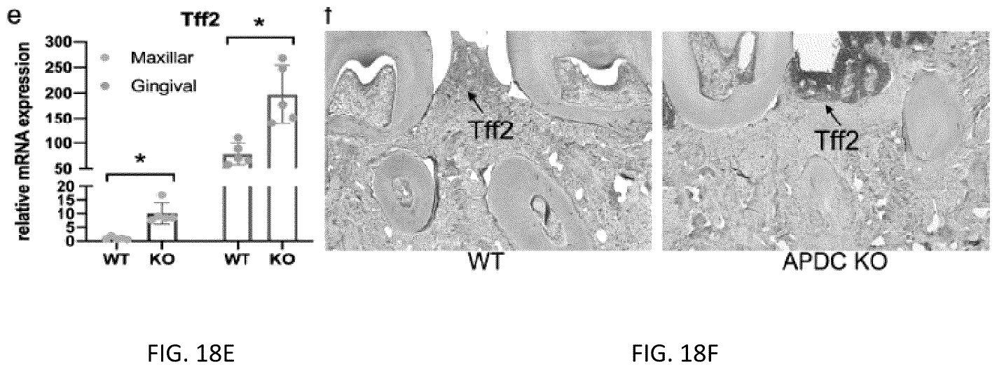

Upon performing DEG gene ranking on all cell types in the gingival, we observed that the Tff2 gene was the top upregulated gene across all cell types in the APDC-KO group (FIG. 15, FIG. 17). The UMP and volcano plot represent the differential expression levels of Tff2 between the IncR-APDC-KO and wild-type groups (FIG. 18A, FIG. 18B). Tff2 belongs to the trefoil factor family (TFFs) and is predominantly expressed in the stomach (50). Research regarding trefoil factors is an emerging area of investigation in the dental field. TFFs have been reported to be expressed in oral mucosal cells, with a novel role in chronic inflammatory conditions related to mucosal healing and function restoration. The TFFs expression have been detected in saliva and gingival samples presenting severe PD (51). To reveal the underlying mechanism between APDC deficiency and the high expression of Tff2, we further investigated the interaction of APDC and Tff2 using IntaRNA; this tool enables accurate prediction of RNA-RNA hybrids by incorporating seed constraints and interaction site accessibility (52). The RNA sequence of APDC (AK148321, 2069 bp) and Tff2 (NM_009363.3, 571 bp) were processed. The binding site with −25.07 kcal/mol interaction energy, close to 100% portability was found at the location of 1986-2026, close to 3′ UTR of APDC (FIG. 18C, FIG. 18D), which indicates the direct interaction of the two RNAs. We proceeded to substantiate that the RNA expression level of Tff2 was notably elevated in both maxillary bone and gingival tissues (FIG. 18E). Furthermore, we observed that TFF2 protein exhibited high expression levels specifically within the APDC-KO periodontitis samples (FIG. 18F).

### Cell-Cell Communications Among Different Cell Types in the Inflamed Gingival Tissue

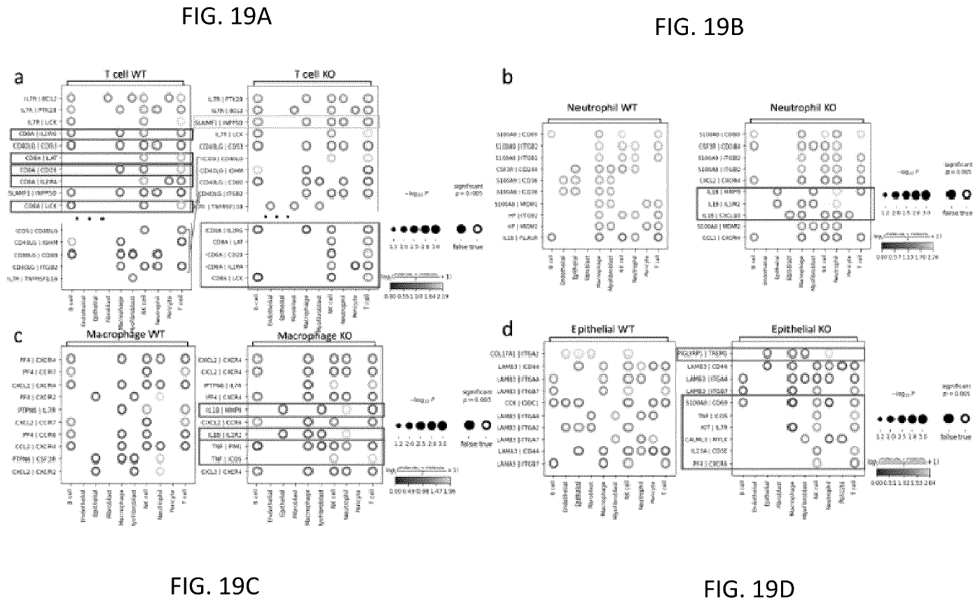

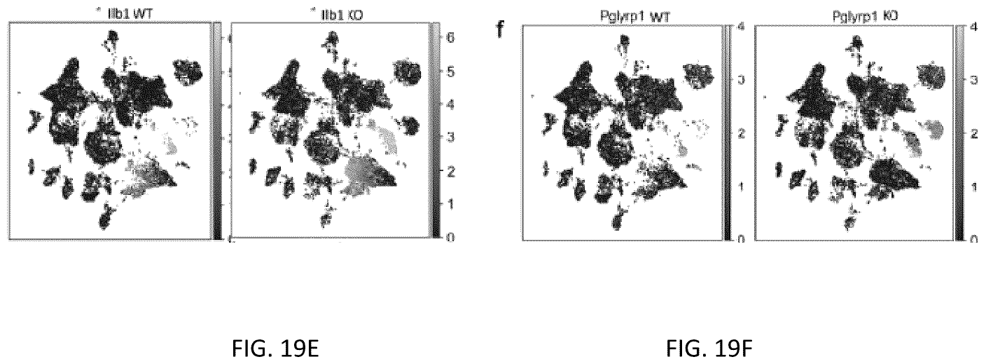

We performed a computational analysis to identify biologically relevant interacting ligand-receptor partners from our scRNA-seq data (see Methods). In the wild-type group, T cells communicated with B cells, macrophages, and NK cells through the ligand of CD8A. However, in the APDC-KO group, these interactions were significantly weakened, and the CD40 ligand (CD40LG) became the predominant mode of communication between T cells and other immune cells (FIG. 19A). These changes might be due to the lack of CD8+ cells in the APDC-KO group. In addition, the “SLAMF1-INPP5D” pair, associated with suppressed IFN-γ production in T-cells, showed a significant upregulation in the APDC-KO group.

Within the APDC-KO group, macrophages and neutrophils displayed heightened communication with T cells, epithelial cells, fibroblasts, and among themselves through IL1β (FIG. 19B, FIG. 19C). The KO macrophages and neutrophils consistently higher expressed IL1β compared to the wild type (FIG. 19E). In addition, the KO macrophages interacted with T and NK cells through TNF-PIM1 and TNF-ICOS (FIG. 16C). The PGLYRP1-TREM1 interaction was extremely active in the APDC-KO gingival tissue between epithelial cells and macrophages as well as neutrophils (FIG. 19D). Interestingly, Pglyrp1 was highly expressed in neutrophils and the subtype of epithelial cells (Krt8+, Krt18+, Krt5−, Krt14−) that we identified in the APDC-KO gingival (FIG. 19F). It has been shown that peptidoglycan recognition protein 1 (PGLYRP1) forms a Trem1 ligand complex with PGN, and stimulates cytokine production in neutrophils and macrophages (53). Furthermore, TREM1/PGLYRP1/IL1p signaling pathway regulation is related to the response to bacterial biofilm accumulation and removal in PD (54). Taken together, the upregulation of the TREM1/PGLYRP1/IL1p axis indicates a new mechanism describing how APDC deficiency causes a faster and more excessive immune response to the pathogen stimulation and leads to more severe periodontal inflammation and bone loss.

### AAV9-CAG-APDC Significantly Ameliorates Periodontitis

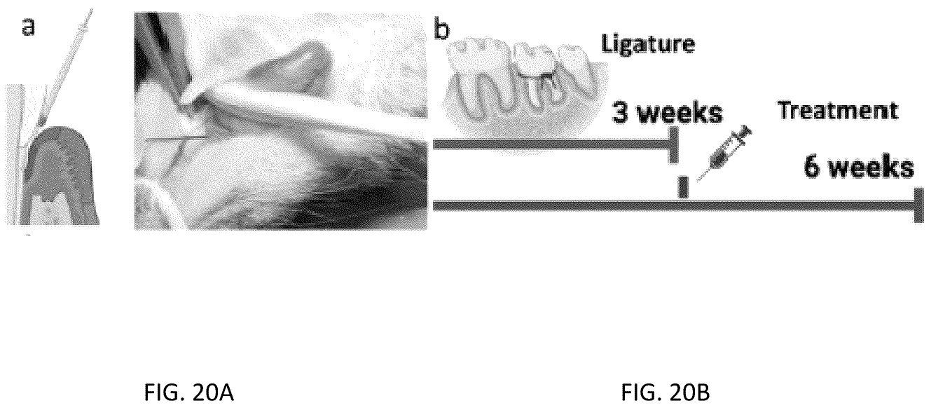

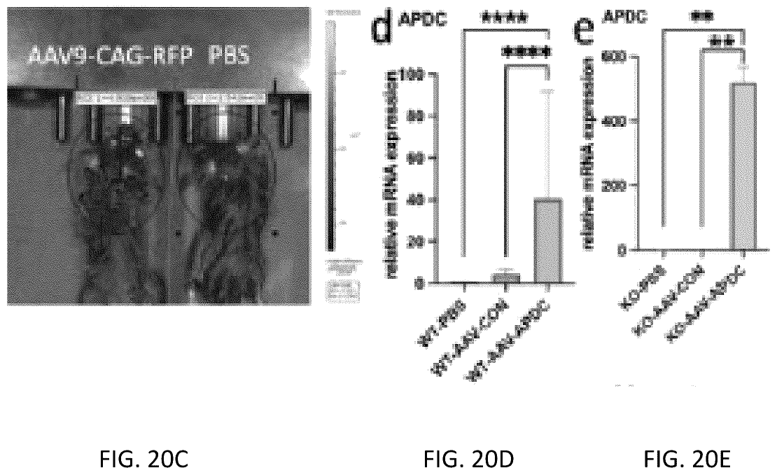

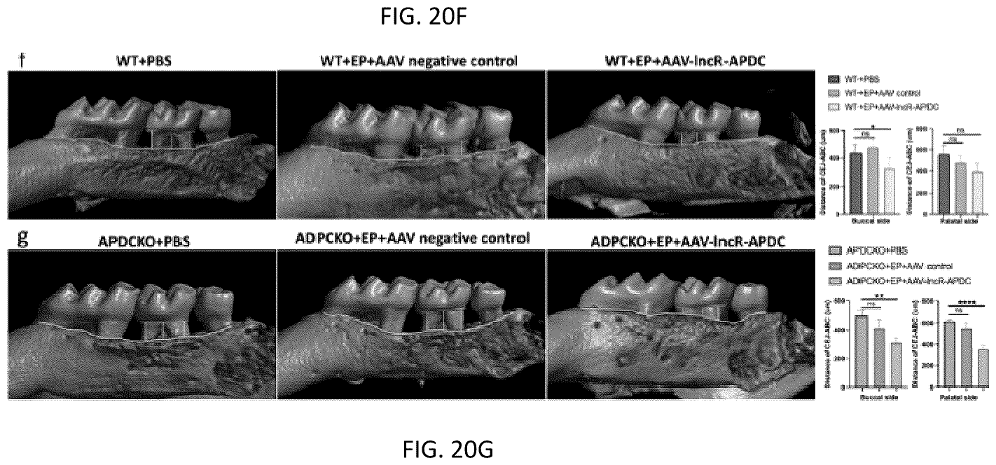

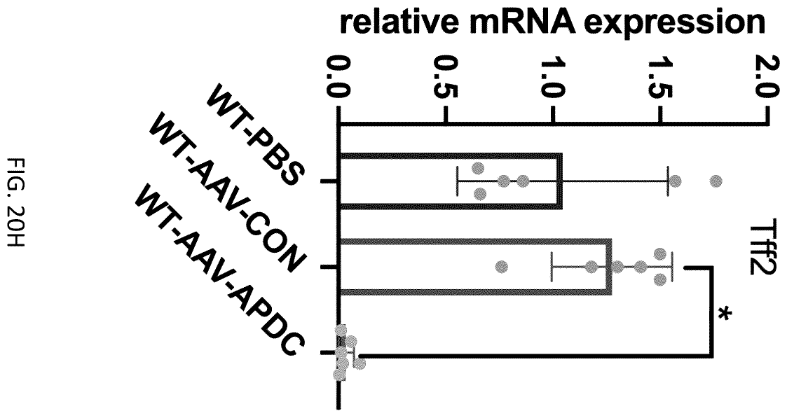

To assess the therapeutic impact of APDC in periodontitis, we constructed the adeno-associated virus 9-CAG-lncR-APDC (AAV9-APDC). EP was induced on 4-month-old female 129SVE (wild-type) and APDC-KO mice (6 groups, n=5-6 per group). The ligatures were removed after 3 weeks. AAV9-APDC was then administered through gingival microinjection to evaluate its ability to alleviate bone loss resulting from periodontitis. The AAV9 empty vector was used as the negative control (FIG. 20A, FIG. 20B). IVIS Spectrum CT imaging confirmed successful AAV9 delivery through local injection, as visualized by the expression of red fluorescent protein (RFP) (FIG. 20C). Furthermore, we evaluated the expression level of APDC and observed significantly elevated expression in the AAV9-APDC delivery groups (FIG. 20D, FIG. 20E). Remarkably, the WT-EP-AAV9-APDC group exhibited significant reduction in bone loss and a notable decrease in Tff2 level (FIG. 20F). Intriguingly, the alveolar bone loss in the KO-EP-AAV-APDC group was also significantly recovered and was close to the same level as the wild-type group with AAV-APDC treatment (FIG. 20G). Compared to the wild-type group, the Tff2 level was considerably higher in the KO-PBS group. However, following the administration of AAV-APDC this elevation was notably reduced (FIG. 20H, FIG. 20I).

### Discussion

In the present study, we demonstrated that APDC deficiency exacerbates periodontitis by increasing alveolar bone loss and inflammatory infiltration of the periodontal tissue. The BMSCs harvested from lncR-APDC-KO mice showed downregulated osteogenic differentiation. Single cell RNA sequencing analysis of the gingival tissue from the EP model revealed that the deletion of IncR-APDC reduces the population of B cells and CD8+ cytotoxic T cells, while increasing the M1/M2 ratio and the recruitment of neutrophils to the inflamed periodontal tissues. Furthermore, a novel subtype of epithelial cells, characterized by a distinct gene profile (Krt8+ and Krt18+), and actively communicating with macrophages and neutrophils through the PGLYRP1-TREM1 pairing, was identified exclusively in the IncR-APDC-KO group. We also discovered that Tff2 is the most highly expressed gene among the DEGs in all cell types of the inflamed lncR-APDC-KO gingival tissue, and predicted the direct binding of Tff2 and lncR-APDC. Lastly, we successfully attenuated periodontal bone loss by delivering AAV9-CAG-APDC to the experimental periodontitis site, which shows the great potential of IncR-APDC in the treatment of periodontal disease.

After data filtering, we analyzed 25,161 single cells (13,244 from APDC-KO mice, and 11,917 from control mice), which is a satisfactory amount of high-quality data for scRNA seq. However, for the cell types with relatively small populations, there were still challenges when further dividing their subtypes. For example, we sub-clustered T cells into memory CD4 and CD8 T cells, as well as naïve CD4 and CD8 T cells based on the expression level of Cd4, Cd8a, Sell and Cd44. With more biological replicates, the enlarged pool of T cells would allow us to further divide the CD8+ T cells into Tc1, Tc2, Tc9, Tc17 and Tc22 T cells (55), and reveal more changes in the immune function of CD8+ T cells. Furthermore, NK cells are abundant in periodontitis lesions, and their activation has been causally linked to periodontal tissue destruction. In our scRNA seq analysis, NK cells were detected in both groups, though no significant difference was observed (FIG. 15). In our future studies, we plan to acquire more data to thoroughly isolate and characterize these cell types. Additionally, we aim to integrate multi-omics approaches to comprehensively analyze the molecular signatures and signal pathways.

The wildly upregulated Tff2 in the APDC deficient mice offers new insights into the functions of APDC. Tff2 was originally found in the gastric mucosal lining, small and large intestine, oral mucosal cells, and salivary glands. At present, TFF expression has been detected in severe periodontal diseased tissue samples (51,56). Mahendra et al. reported markedly heightened levels of TFF2 protein in saliva samples from patients diagnosed with coronary heart disease (CHD) and PD (57). The discovery of the direct binding site of APDC and Tff2 provides us a new direction to reveal APDC's regulatory mechanism in PD. In addition, the Mucin5b (Muc5b) gene was also significantly upregulated in many cell types in the APDC-KO group (FIG. 15, FIG. 17). This gene is specifically involved in mucosal innate immunity and is commonly co-expressed with the TFF family. We will perform further investigation into Tff2, Muc5b and related pathways in our forthcoming investigations.

Our study revealed that APDC deficiency leads to alterations in the interaction network between immune cells and other cell types in the gingival tissue. For example, we show that (1) the reduced interactions involving CD8A as a ligand (e.g., CD8A-IL2RA, CD8A-IL2RG and CD8A-CD28), between T cells and other immune cells, can be attributed to the absence of CD8+ cells in the APDC-KO group; and (2) the TREM1/PGLYRP1/IL1p axis is upregulated in the KO group and actively involved in epithelial cell, macrophage, and neutrophil interactions. Utilizing single-cell RNA sequencing data for ligand-receptor pairing analysis is a well-established method (58-60). Our results demonstrate the application of this method in studying periodontitis progression.

The incRNAs have great potential in clinical application. There are currently 73 completed and ongoing clinical trials using lncRNAs as markers and therapeutic targets (clinicaltrials.gov). Notably, only two of these trials are focused on oral disease. Our understanding of the roles of lncRNAs in dental diseases is still in the early stages of exploration. The differentially expressed lncRNAs have been identified in oral lichen planus (OLP), oral submucous fibrosis (OSF), oral squamous cell carcinoma (OSCC), Sjögren's syndrome and periodontitis patients (61-67). However, the underlying epigenetic mechanisms governing the regulatory functions of these lncRNAs remain inconclusive. Furthermore, ANRIL is the shared risk factor of many chronic inflammatory diseases in humans. Besides CAD, PD, and diabetes, ANRIL is also involved in chronic obstructive pulmonary disease (68) and uric acid nephropathy (69). The analysis of APDC's regulatory role on immune response, intercellular communication among immune cells, and common pro-inflammation cytokines from this study, could provide fundamental knowledge for researching ANRIL and APDC related systemic inflammation diseases mentioned above.

This paper presents the first comprehensive study on the influence of IncR-APDC in bone resorption and immune response within inflamed periodontal tissue. It highlights changes in the ratio and functionality of immune and epithelial cells, and uncovers a distinctive epithelial subcluster and elevated Tff2 expression related to APDC deficiency during periodontitis. The findings suggest that lncR-APDC holds significant potential for the prevention and treatment of PD, as well as other chronic inflammatory conditions.

In summary, this study suggests that lncR-APDC is a critical player in the pathogenesis of PD and may offer therapeutic potential. Additionally, the identification of unique epithelial cell subsets and the interaction between lncR-APDC and Tff2 open new avenues for understanding the epigenetic regulation of periodontitis. Further studies in this area could lead to innovative approaches for managing and treating PD.

### REFERENCES FOR EXAMPLE 2

1. Periodontal Disease|Oral Health Conditions|Division of Oral Health|CDC [Internet]. 2018. Available from: cdc.gov/oralhealth/conditions/periodontal-disease
2. Abusleme L, Hoare A, Hong B Y, Diaz P I. Microbial signatures of health, gingivitis, and periodontitis. Periodontol 2000. 2021 June; 86(1):57-78.
3. Hajishengallis G, Chavakis T. Local and systemic mechanisms linking periodontal disease and inflammatory comorbidities. Nat Rev Immunol. 2021 July; 21(7):426-40.
4. Sanz M, Marco Del Castillo A, Jepsen S, Gonzalez-Juanatey J R, D'Aiuto F, Bouchard P, et al. Periodontitis and cardiovascular diseases: Consensus report. J Clin Periodontol. 2020 March; 47(3):268-88.
5. Liccardo D, Cannavo A, Spagnuolo G, Ferrara N, Cittadini A, Rengo C, et al. Periodontal Disease: A Risk Factor for Diabetes and Cardiovascular Disease. Int J Mol Sci. 2019 Mar. 20; 20(6):1414.
6. Kinane D F, Stathopoulou P G, Papapanou P N. Periodontal diseases. Nat Rev Dis Primers. 2017 Jun. 22; 3:17038.
7. Peng W X, Koirala P, Mo Y Y. LncRNA-mediated regulation of cell signaling in cancer. Oncogene. 2017 Oct. 12; 36(41):5661-7.
8. Huang Y. The novel regulatory role of lncRNA-miRNA-mRNA axis in cardiovascular diseases. J Cell Mol Med. 2018 December; 22(12):5768-75.
9. Rahimi E, Ahmadi A, Boroumand M A, Mohammad Soltani B, Behmanesh M. Association of ANRIL Expression with Coronary Artery Disease in Type 2 Diabetic Patients. Cell J. 2018; 20(1):41-5.
10. Kong Y, Hsieh C H, Alonso L C. ANRIL: A lncRNA at the CDKN2A/B Locus With Roles in Cancer and Metabolic Disease. Frontiers in Endocrinology [Internet]. 2018; 9. Available from: frontiersin.org/articles/10.3389/fendo.2018.00405
11. Razeghian-Jahromi I, Karimi Akhormeh A, Zibaeenezhad M J. The Role of ANRIL in Atherosclerosis. Disease Markers. 2022 Feb. 9; 2022:e8859677.
12. Bochenek G, Hasler R, El Mokhtari N E, Konig I R, Loos B G, Jepsen S, et al. The large non-coding RNA ANRIL, which is associated with atherosclerosis, periodontitis and several forms of cancer, regulates ADIPOR1, VAMP3 and C11ORF10. Hum Mol Genet. 2013 Nov. 15; 22(22):4516-27.
13. Schaefer A S, Richter G M, Groessner-Schreiber B, Noack B, Nothnagel M, El Mokhtari N E, et al. Identification of a shared genetic susceptibility locus for coronary heart disease and periodontitis. PLoS Genet. 2009 February; 5(2):e1000378.
14. Aarabi G, Zeller T, Heydecke G, Munz M, Schafer A, Seedorf U. Roles of the Chr.9p21.3 ANRIL Locus in Regulating Inflammation and Implications for Anti-Inflammatory Drug Target Identification. Frontiers in Cardiovascular Medicine [Internet]. 2018; 5. Available from: frontiersin.org/articles/10.3389/fcvm.2018.00047
15. Santos C M M L, Lira-Junior R, Fischer R G, Santos A P P, Oliveira B H. Systemic Antibiotics in Periodontal Treatment of Diabetic Patients: A Systematic Review. PLoS One. 2015 Dec. 22; 10(12):e0145262.
16. Gholami L, Ghafouri-Fard S, Mirzajani S, Arsang-Jang S, Taheri M, Dehbani Z, et al. The lncRNA ANRIL is down-regulated in peripheral blood of patients with periodontitis. Noncoding RNA Res. 2020 Apr. 17; 5(2):60-6.
17. Schaefer A S, Richter G M, Dommisch H, Reinartz M, Nothnagel M, Noack B, et al. CDKN2BAS is associated with periodontitis in different European populations and is activated by bacterial infection. Journal of Medical Genetics. 2011 Jan. 1; 48(1):38-47.
18. Liu X, Zhou Y. Downregulation of lncRNA ANRIL Inhibits Osteogenic Differentiation of Periodontal Ligament Cells via Sponging miR-7 through NF-κB Pathway. Anal Cell Pathol (Amst). 2021 Nov. 24; 2021:7890674.
19. Teeuw W J, Laine M L, Bizzarro S, Loos B G. A Lead ANRIL Polymorphism Is Associated with Elevated CRP Levels in Periodontitis: A Pilot Case-Control Study. PLOS ONE. 2015 Sep. 8; 10(9):e0137335.
20. Visel A, Zhu Y, May D, Afzal V, Gong E, Attanasio C, et al. Targeted deletion of the 9p21 non-coding coronary artery disease risk interval in mice. Nature. 2010 March; 464(7287):409-12.
21. Gao S, Cheng Q C, Hu Y G, Tan Z Z, Chen L, Liu S W, et al. LncRNA AK148321 alleviates neuroinflammation in LPS-stimulated BV2 microglial cell through regulating microRNA-1199-5p/HSPA5 axis. Life Sciences. 2021 Feb. 1; 266:118863.
22. Loinard C, Basatemur G, Masters L, Baker L, Harrison J, Figg N, et al. Deletion of Chromosome 9p21 Noncoding Cardiovascular Risk Interval in Mice Alters Smad2 Signaling and Promotes Vascular Aneurysm. Circulation: Cardiovascular Genetics. 2014 December; 7(6):799-805.
23. Pan W, Wang Q, Chen Q. The cytokine network involved in the host immune response to periodontitis. Int J Oral Sci. 2019 Nov. 5; 11(3):1-13.
24. Ramadan D E, Hariyani N, Indrawati R, Ridwan R D, Diyatri I. Cytokines and Chemokines in Periodontitis. Eur J Dent. 2020 July; 14(3):483-95.
25. Behl Y, Siquiera M, Ortiz J, Desta T, Faibish D, Graves D T. Activation of the Acquired Immune Response Reduces Coupled Bone Formation in Response to a Periodontal Pathogen. J Immunol. 2008 Dec. 15; 181(12):8711-8.
26. Wolf F A, Angerer P, Theis F J. SCANPY: large-scale single-cell gene expression data analysis. Genome Biology. 2018 Feb. 6; 19(1):15.
27. Chen Y, Wang H, Yang Q, Zhao W, Chen Y, Ni Q, et al. Single-cell RNA landscape of the osteoimmunology microenvironment in periodontitis. Theranostics. 2022 Jan. 1; 12(3):1074-96.
28. Williams D W, Greenwell-Wild T, Brenchley L, Dutzan N, Overmiller A, Sawaya A P, et al. Human oral mucosa cell atlas reveals a stromal-neutrophil axis regulating tissue immunity. Cell. 2021 Jul. 22; 184(15):4090-4104.e15.
29. Cao Y, Wang X, Peng G. SCSA: A Cell Type Annotation Tool for Single-Cell RNA-seq Data. Frontiers in Genetics [Internet]. 2020; 11. Available from: frontiersin.org/article/10.3389/fgene.2020.00490
30. Alvarez C, Abdalla H, Suliman S, Rojas P, Wu Y C, Almarhoumi R, et al. RvE1 Impacts the Gingival Inflammatory Infiltrate by Inhibiting the T Cell Response in Experimental Periodontitis. Frontiers in Immunology [Internet]. 2021; 12. Available from:
   frontiersin.org/articles/10.3389/fimmu.2021.664756
31. Golubovskaya V, Wu L. Different Subsets of T Cells, Memory, Effector Functions, and CAR-T Immunotherapy. Cancers (Basel). 2016 Mar. 15; 8(3):36.
32. Sckisel G D, Mirsoian A, Minnar C M, Crittenden M, Curti B, Chen J Q, et al. Differential phenotypes of memory CD4 and CD8 T cells in the spleen and peripheral tissues following immunostimulatory therapy. Journal for ImmunoTherapy of Cancer. 2017 Apr. 18; 5(1):33.
33. Cardoso E M, Arosa F A. CD8+ T Cells in Chronic Periodontitis: Roles and Rules. Front Immunol. 2017 Feb. 21; 8:145.
34. Choi Y, Woo K M, Ko S H, Lee Y J, Park S J, Kim H M, et al. Osteoclastogenesis is enhanced by activated B cells but suppressed by activated CD8(+) T cells. Eur J Immunol. 2001 July; 31(7):2179-88.
35. Cyster J G, Allen C D C. B cell responses—Cell interaction dynamics and decisions. Cell. 2019 Apr. 18; 177(3):524-40.
36. Figueredo C M, Lira-Junior R, Love R M. T and B Cells in Periodontal Disease: New Functions in A Complex Scenario. Int J Mol Sci. 2019 Aug. 14; 20(16):3949.
37. Shen Y, Ma Y, Xie J, Lin L, Shi Y, Li X, et al. A regulatory role for CD72 expression on B cells and increased soluble CD72 in primary Sjogren's syndrome. BMC Immunology. 2020 Apr. 19; 21(1):21.
38. Spivia W, Magno P S, Le P, Fraser D A. Complement protein C1q promotes macrophage anti-inflammatory M2-like polarization during the clearance of atherogenic lipoproteins. Inflamm Res. 2014 October; 63(10):885-93.
39. Han Y, Huang Y, Gao P, Yang Q, Jia L, Zheng Y, et al. Leptin Aggravates Periodontitis by Promoting M1 Polarization via NLRP3. J Dent Res. 2022 June; 101(6):675-85.
40. Rosales C. Neutrophil: A Cell with Many Roles in Inflammation or Several Cell Types?Frontiers in Physiology [Internet]. 2018; 9. Available from: frontiersin.org/articles/10.3389/fphys.2018.00113
41. Jiang Q, Zhao Y, Shui Y, Zhou X, Cheng L, Ren B, et al. Interactions Between Neutrophils and Periodontal Pathogens in Late-Onset Periodontitis. Frontiers in Cellular and Infection Microbiology [Internet]. 2021; 11. Available from:
   frontiersin.org/articles/10.3389/fcimb.2021.627328
42. Mortaz E, Alipoor S D, Adcock I M, Mumby S, Koenderman L. Update on Neutrophil Function in Severe Inflammation. Front Immunol. 2018 Oct. 2; 9:2171.
43. Del Prete A, Martínez-Muñoz L, Mazzon C, Toffali L, Sozio F, Za L, et al. The atypical receptor CCRL2 is required for CXCR2-dependent neutrophil recruitment and tissue damage.

### Blood. 2017 Sep. 7; 130(10):1223-34.

44. Qian S jiao, Huang Q ru, Chen R ying, Mo J ji, Zhou L yi, Zhao Y, et al. Single-Cell RNA Sequencing Identifies New Inflammation-Promoting Cell Subsets in Asian Patients With Chronic Periodontitis. Front Immunol. 2021 Sep. 8; 12:711337.
45. Smith P C, Martínez C, Martínez J, McCulloch C A. Role of Fibroblast Populations in Periodontal Wound Healing and Tissue Remodeling. Front Physiol. 2019 Apr. 24; 10:270.
46. Smith P. Role of myofibroblasts in normal and pathological periodontal wound healing. Oral Diseases. 2018; 24(1-2):26-9.
47. Darby I A, Laverdet B, Bonté F, Desmoulière A. Fibroblasts and myofibroblasts in wound healing. Clin Cosmet Investig Dermatol. 2014 Nov. 6; 7:301-11.
48. Komaki M. Pericytes in the Periodontal Ligament. Adv Exp Med Biol. 2019; 1122:169-86.
49. Han W, Hu C, Fan Z J, Shen G L. Transcript levels of keratin 1/5/6/14/15/16/17 as potential prognostic indicators in melanoma patients. Sci Rep. 2021 Jan. 13; 11(1):1023.
50. Hoffmann W. Trefoil Factor Family (TFF) Peptides and Their Diverse Molecular Functions in Mucus Barrier Protection and More: Changing the Paradigm. Int J Mol Sci. 2020 Jun. 25; 21(12):4535.
51. Choudhary A, Smitha C N, Suresh D K. Trefoils: An unexplored natural protective shield of oral cavity. J Oral Biol Craniofac Res. 2015; 5(3):226-31.
52. Mann M, Wright P R, Backofen R. IntaRNA 2.0: enhanced and customizable prediction of RNA-RNA interactions. Nucleic Acids Research. 2017 Jul. 3; 45(W1):W435-9.
53. Read C B, Kuijper J L, Hjorth S A, Heipel M D, Tang X, Fleetwood A J, et al. Cutting Edge: Identification of Neutrophil PGLYRP1 as a Ligand for TREM-1. The Journal of Immunology. 2015 Feb. 15; 194(4):1417-21.
54. Silbereisen A, Hallak A K, Nascimento G G, Sorsa T, Belibasakis G N, Lopez R, et al. Regulation of PGLYRP1 and TREM-1 during Progression and Resolution of Gingival Inflammation. JDR Clinical & Translational Research. 2019 Oct. 1; 4(4):352-9.
55. Paul M S, Ohashi P S. The Roles of CD8+ T Cell Subsets in Antitumor Immunity. Trends in Cell Biology. 2020 Sep. 1; 30(9):695-704.
56. Chaiyarit P, Chayasadom A, Wara-Aswapati N, Hormdee D, Sittisomwong S, Nakaresisoon S, et al. Trefoil factors in saliva and gingival tissues of patients with chronic periodontitis. J Periodontol. 2012 September; 83(9):1129-38.
57. Mahendra J, Srinivasan S, Kanakamedala A, D N, Namasivayam A, Mahendra L, et al.

Expression of trefoil factor 2 and 3 and adrenomedullin in chronic periodontitis subjects with coronary heart disease. J Periodontol. 2023 May; 94(5):694-703.
58. Palla G, Spitzer H, Klein M, Fischer D, Schaar A C, Kuemmerle L B, et al. Squidpy: a scalable framework for spatial omics analysis. Nat Methods. 2022 February; 19(2):171-8.
59. Efremova M, Vento-Tormo M, Teichmann S A, Vento-Tormo R. CellPhoneDB: inferring cell-cell communication from combined expression of multi-subunit ligand-receptor complexes. Nat Protoc. 2020 April; 15(4):1484-506.
60. Türei D, Korcsmáros T, Saez-Rodriguez J. OmniPath: guidelines and gateway for literature-curated signaling pathway resources. Nat Methods. 2016 December; 13(12):966-7.
61. Wang Y K, Liu C M, Lin T, Fang C Y, Yu C C, Yu C H. Inhibition of HIF1A-AS1 impedes the arecoline-induced migration activity of human oral mucosal fibroblasts. J Formos Med Assoc. 2020 April; 119(4):879-83.
62. Yang Q, Xu B, Sun H, Wang X, Zhang J, Yu X, et al. A genome-wide association scan of biological processes involved in oral lichen planus and oral squamous cell carcinoma. Medicine (Baltimore). 2017 June; 96(25):e7012.
63. Wang J, Zhai X, Guo J, Li Y, Yang Y, Wang L, et al. Long non-coding RNA DQ786243 modulates the induction and function of CD4+ Treg cells through Foxp3-miR-146a-NF-κB axis: Implications for alleviating oral lichen planus. Int Immunopharmacol. 2019 October; 75:105761.
64. Zhang L, Meng X, Zhu X W, Yang D C, Chen R, Jiang Y, et al. Long non-coding RNAs in Oral squamous cell carcinoma: biologic function, mechanisms and clinical implications. Mol Cancer. 2019 May 27; 18(1):102.
65. Zou Y, Li C, Shu F, Tian Z, Xu W, Xu H, et al. lncRNA expression signatures in periodontitis revealed by microarray: the potential role of lncRNAs in periodontitis pathogenesis. J Cell Biochem. 2015 April; 116(4):640-7.
66. Dolcino M, Tinazzi E, Vitali C, Del Papa N, Puccetti A, Lunardi C. Long Non-Coding RNAs Modulate Sjögren's Syndrome Associated Gene Expression and Are Involved in the Pathogenesis of the Disease. J Clin Med. 2019 Sep. 1; 8(9):E1349.
67. Zhang K, Qiu W, Wu B, Fang F. Long non-coding RNAs are novel players in oral inflammatory disorders, potentially premalignant oral epithelial lesions and oral squamous cell carcinoma (Review). Int J Mol Med. 2020 August; 46(2):535-45.
68. Ge J, Geng S, Jiang H. Long noncoding RNAs antisense noncoding RNA in the INK4 locus (ANRIL) correlates with lower acute exacerbation risk, decreased inflammatory cytokines, and mild GOLD stage in patients with chronic obstructive pulmonary disease. J Clin Lab Anal. 2019 February; 33(2):e22678.
69. Hu J, Wang D, Wu H, Yang Z, Yang N, Dong J. Long non-coding RNA ANRIL-mediated inflammation response is involved in protective effect of rhein in uric acid nephropathy rats. Cell Biosci. 2019 Jan. 17; 9:11.
70. Marchesan J, Girnary M S, Jing L, Miao M Z, Zhang S, Sun L, et al. An experimental murine model to study periodontitis. Nat Protoc. 2018 October; 13(10):2247-67.
71. Busch A, Richter A S, Backofen R. IntaRNA: efficient prediction of bacterial sRNA targets incorporating target site accessibility and seed regions. Bioinformatics. 2008 Dec. 15; 24(24):2849-56.

### Example 3

### Novel Anti-Inflammation and Soft Tissue Healing Effect of Tff2

Notably, we identified a unique interaction between lncRNA-APDC and Trefoil Factor 2 (Tff2). In lncRNA-APDC mice, Tff2 expression is significantly elevated at both RNA and protein levels across various tissue types. Tff2 encodes a small, secreted protein essential for mucosal protection and repair, contributing to epithelial barrier integrity. Its anti-inflammatory properties-including immune modulation and reduction of tissue inflammation-suggest a potential role in mitigating disease progression. We have established both Tff2 overexpression and silencing models in vitro, demonstrating Tff2's anti-inflammatory and wound healing effects on epithelial cells.

### Overexpression of Tff2 Induces Proliferation in Oral Gingival Epithelial Cells

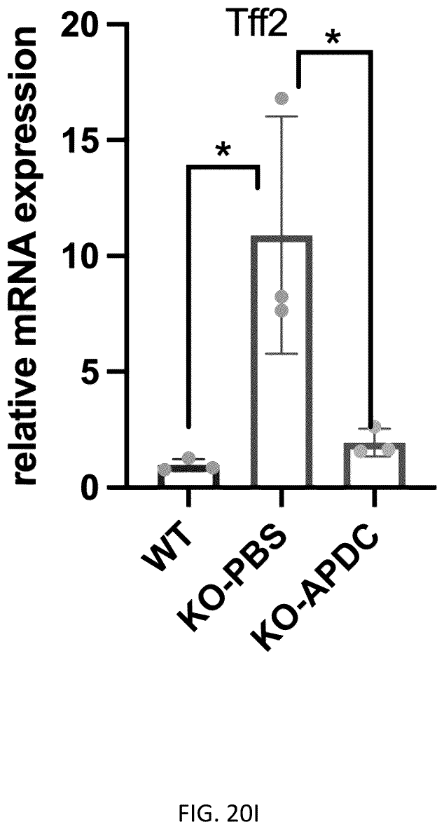

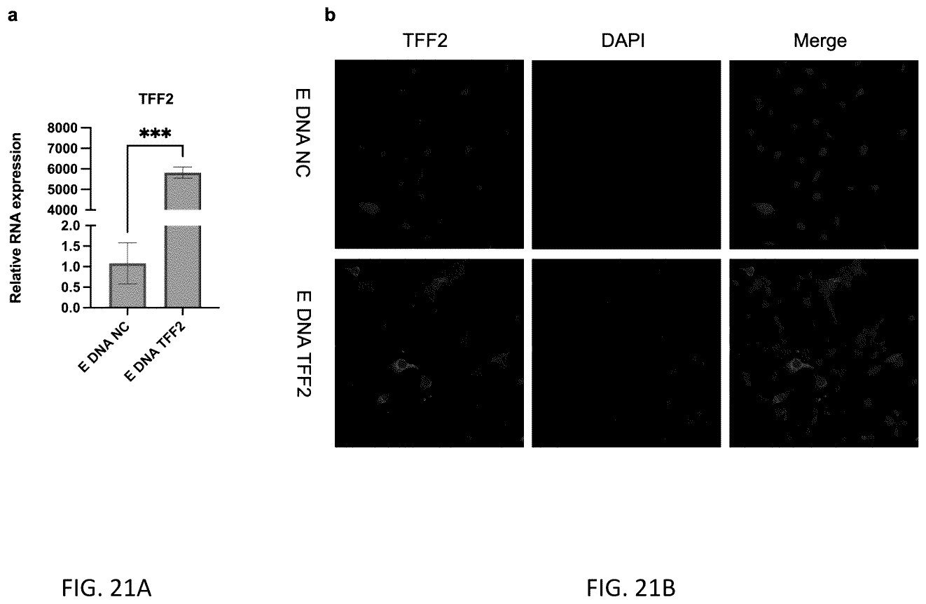

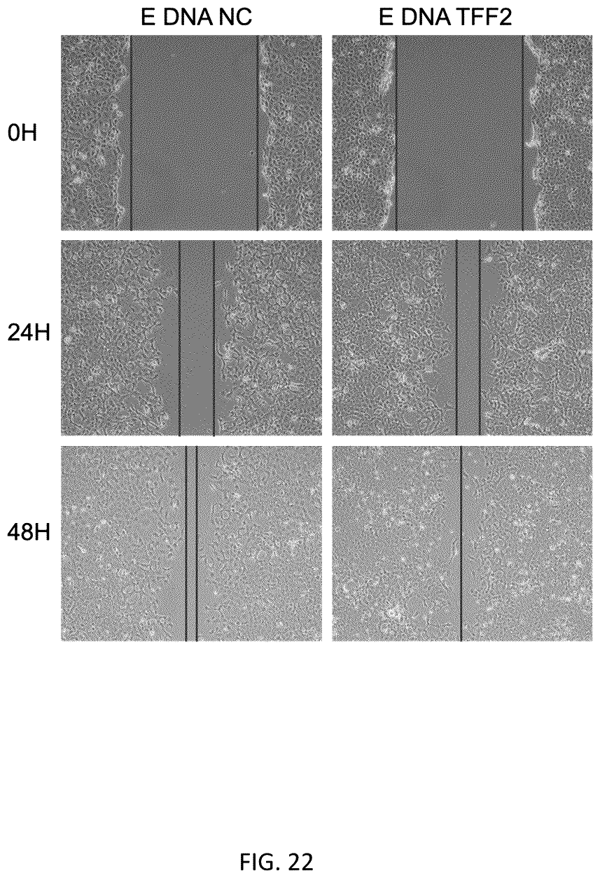

In our previous study, we observed remarkably high expression of Tff2 in APDC-deficient mice in bone and gingiva tissue of our periodontitis mouse model. The trefoil factor family (TFF) consists of small peptides that are predominantly expressed in tissues containing mucus-producing cells, particularly within the mucosa of the gastrointestinal tract. Additionally, Tff2 has been implicated in chronic inflammatory conditions affecting oral mucosal cells and salivary glands. To further explore the role of Tff2 in the chronic inflammation characteristic of periodontitis, we transfected primary gingival epithelial cells with a Tff2-encoding plasmid (epithelial DNA, also referred to as E DNA, Tff2 cell).Subsequent analyses via real-time PCR (FIG. 21A) and immunofluorescence (FIG. 21B) demonstrated significantly elevated Tff2 expression in these cells compared to controls, which received only an empty plasmid (E DNA NC cell). A wound healing assay was conducted to evaluate cell proliferation between the two groups. Cells were cultured in 6-well plates until they reached approximately 90% confluency. A wound was created across the cell monolayer using a 10 μL pipette tip, and detached cells were removed by gently washing with PBS. The culture medium was replaced with serum-free media to ensure proliferation was the primary factor in wound closure. Images of the wound were captured at 0 hours (immediately after wounding), 24 hours, and 48 hours post wounding. The results demonstrated that TFF2 expression promoted oral epithelial cell proliferation, as reflected in the accelerated wound closure observed (FIG. 22).

### Enhancing Anti-Inflammatory Responses in Gingival Epithelial Cells Through Tff2 Overexpression

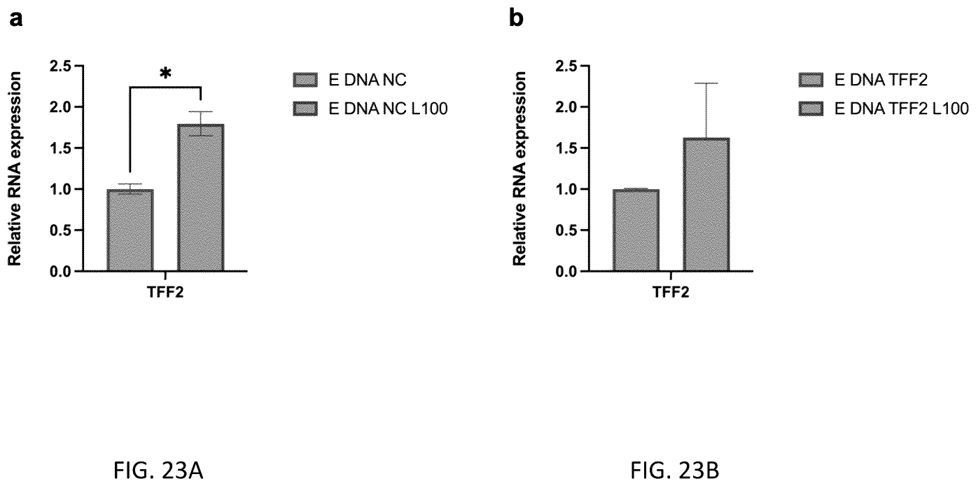

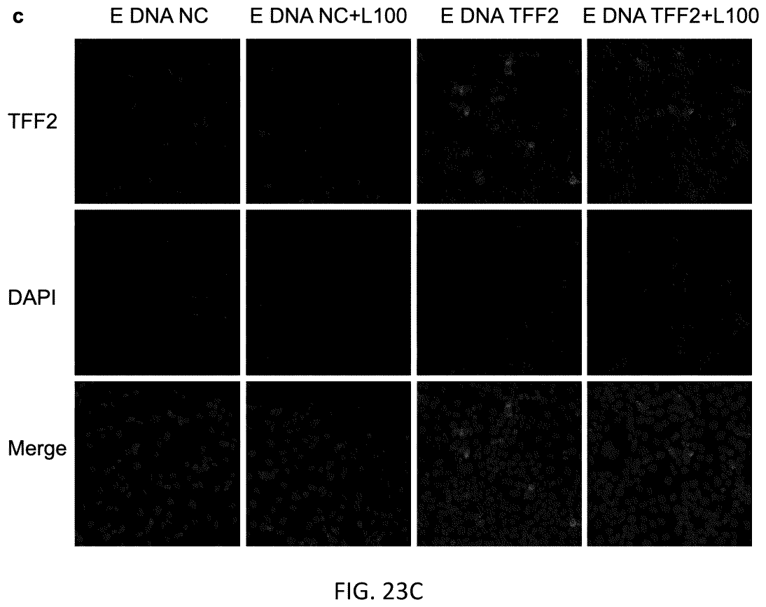

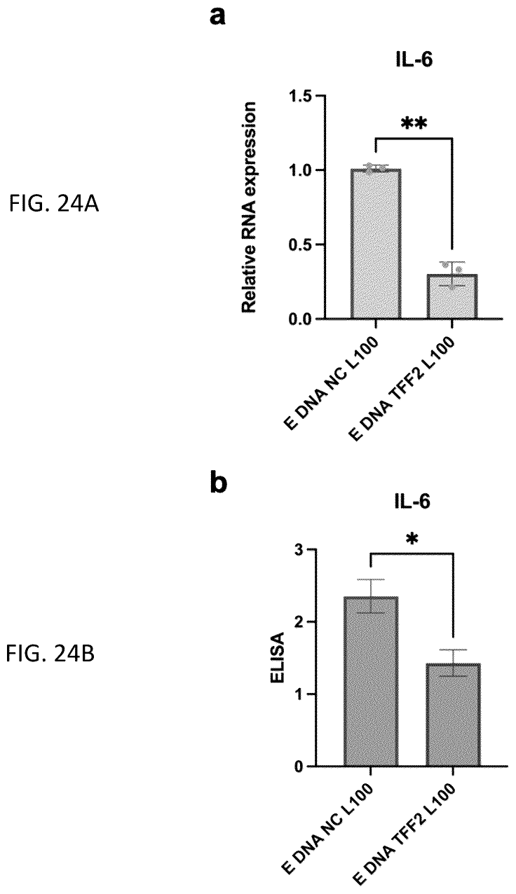

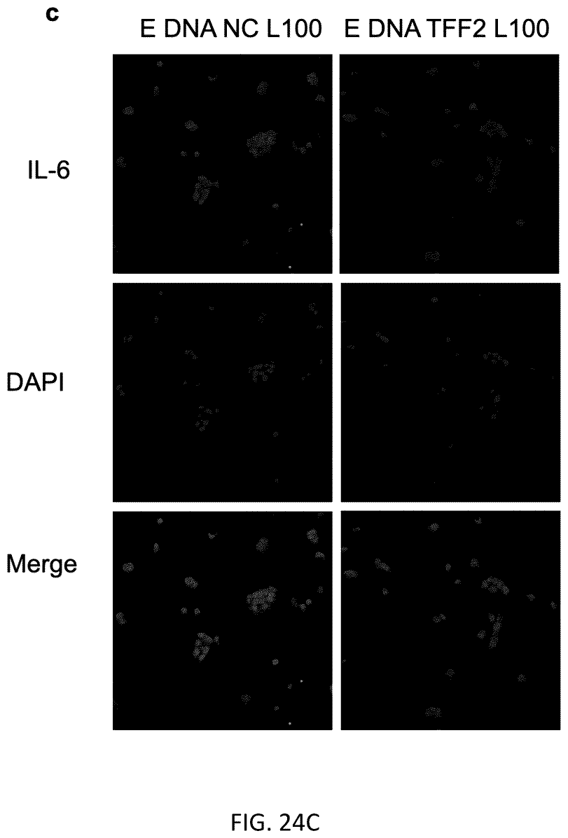

To further elucidate the anti-inflammatory role of Tff2 in periodontal disease, gingival epithelial cells were treated with Porphyromonas gingivalis lipopolysaccharide (Pg. LPS). Real-time PCR analysis revealed that Tff2 expression in E DNA NC cells was up-regulated following Pg. LPS treatment (E DNA NC L100 cells) (FIG. 23A); however, the immunofluorescence (IF) analysis indicated that the changes in Tff2 expression were minimal (FIG. 23C). In contrast, although LPS treatment in E DNA Tff2 cells also led to an up-regulation of TFF2, this increase was not statistically significant (FIG. 23B). Interestingly, IF analysis demonstrated a reduction in Tff2 expression post-LPS treatment (E DNA Tff2 L100 cells, 23C). More importantly, overexpression of Tff2 in these cells was associated with a decrease in interleukin 6 (IL-6) release, both intracellularly and extracellularly, suggesting that Tff2 overexpression may attenuate IL-6 release in an inflammatory environment (FIGS. 24A-24B). This indicates a potential mechanism by which Tff2 confers resistance to inflammatory effects in epithelial cells.

### Silencing the Expression of Tff2 Reduced the Proliferation of Gingival Epithelial Cells

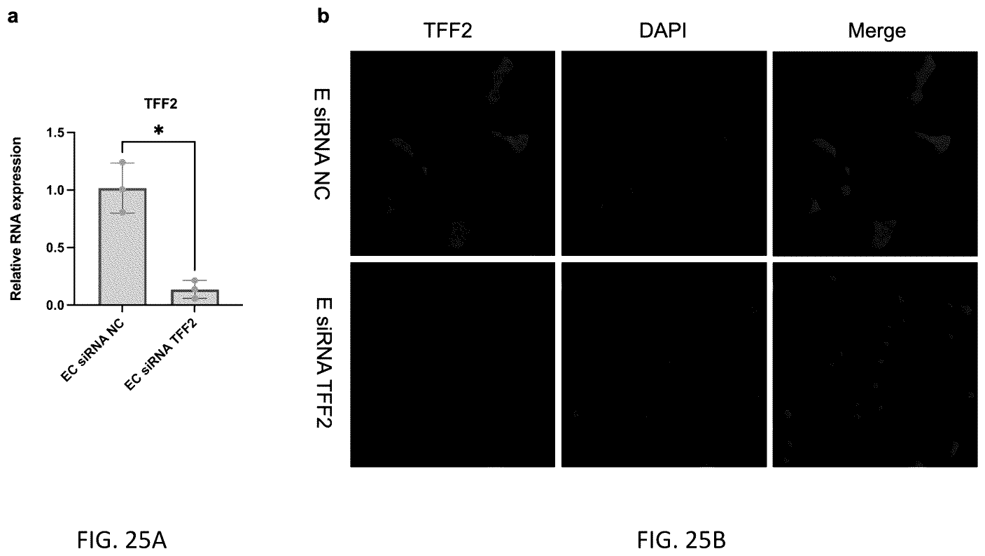

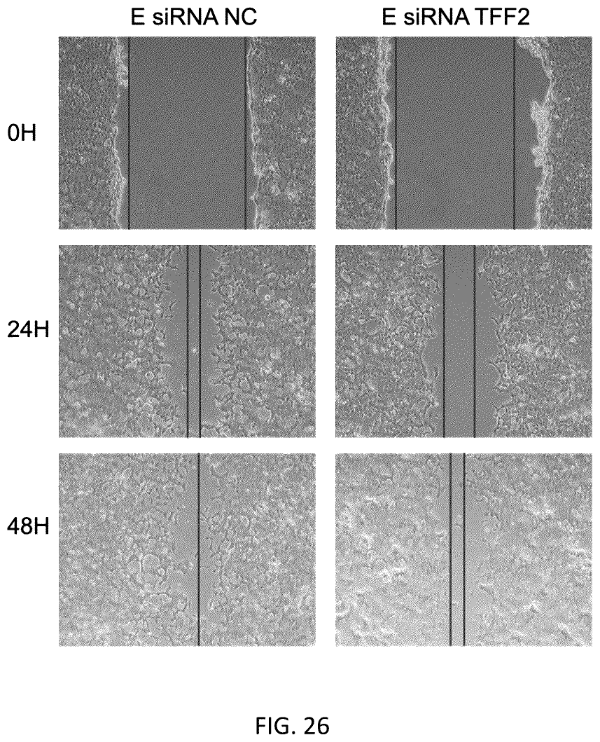

Then, we further investigated the role of Tff2 in cell proliferation after silencing its expression in gingival epithelial cells. As depicted in FIGS. 25A-25B, Tff2 expression was successfully reduced using siRNA transfection (E siRNA Tff2 cells) (siRNA sense strand: GGAUGCUGCUUUGACUCUATT (SEQ ID NO: 95); antisense strand: UAGAGUCAAAGCAGCAUCCTT (SEQ ID NO: 96)). Moreover, silenced Tff2 expression was associated with a decrease in the cell proliferation and migration of oral epithelial cells, as demonstrated in FIG. 26. This indicates that Tff2 has therapeutic potential for periodontal diseases.

### Tff2 in Epithelial Cells May Regulate Muc6 and MMP3, Affecting Wound Healing and Repair

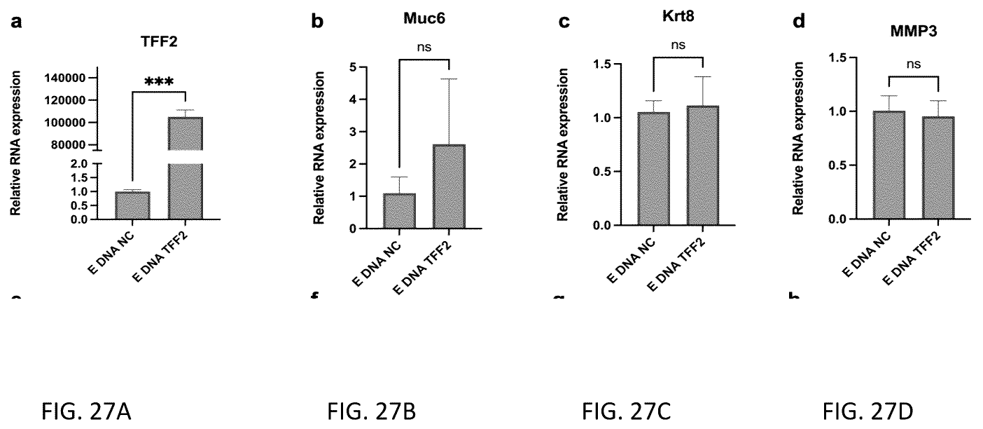

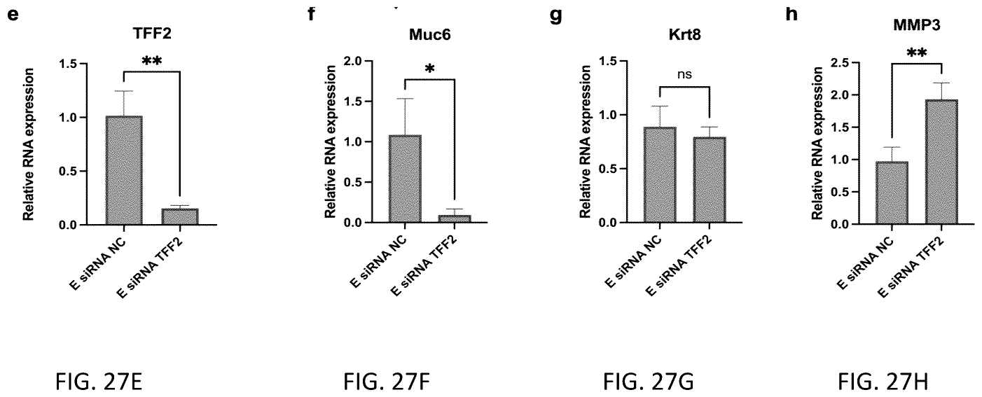

To further elucidate the role of Tff2 in epithelial healing and repair, we performed Real-time PCR analysis on cells with either overexpressed or silenced Tff2. The results revealed a positive correlation between Tff2 expression (FIG. 27A, FIG. 27E) and Muc6 (FIG. 27B, FIG. 27F), a gene intimately associated with epithelial healing. Changes in Krt8 expression were not substantial (FIG. 27C, FIG. 27G). While the upregulation of MMP3 in cells with overexpressed Tff2 was not pronounced, a significant elevation in MMP3 levels was observed in cells with silenced Tff2 (FIG. 27D, FIG. 27H). These findings suggest that Tff2 may influence epithelial healing and repair processes through the modulation of MUC6 and MMP3.

We focused on exploring the role of Tff2 in periodontitis, particularly its involvement in cell proliferation, anti-inflammatory responses, and wound healing. We found that overexpression of Tff2 significantly increased the proliferation and migration of oral gingival epithelial cells, supporting its role in enhancing epithelial regeneration. Additionally, Tff2 overexpression reduced interleukin-6 (IL-6) release, suggesting its anti-inflammatory potential. Conversely, silencing Tff2 expression led to a decrease in cell proliferation. Furthermore, Tff2 appeared to regulate genes associated with epithelial healing, such as MUC6 and MMP3, implicating it in tissue repair processes. These findings highlight Tff2 as a key modulator in both inflammation and wound healing in the context of periodontitis, providing a foundation for further investigation into its therapeutic potential.

### Tff2 May Regulate the MAPK Signaling Pathway in Oral Wound Healing and Repair

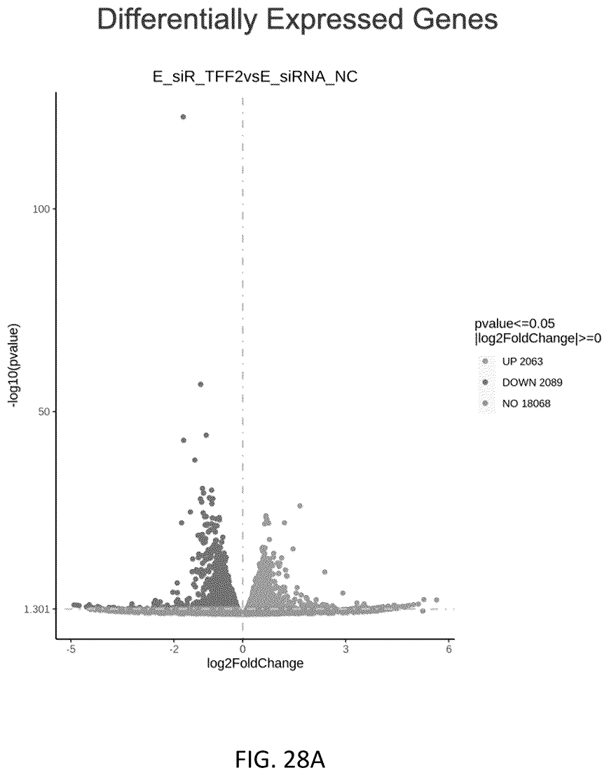

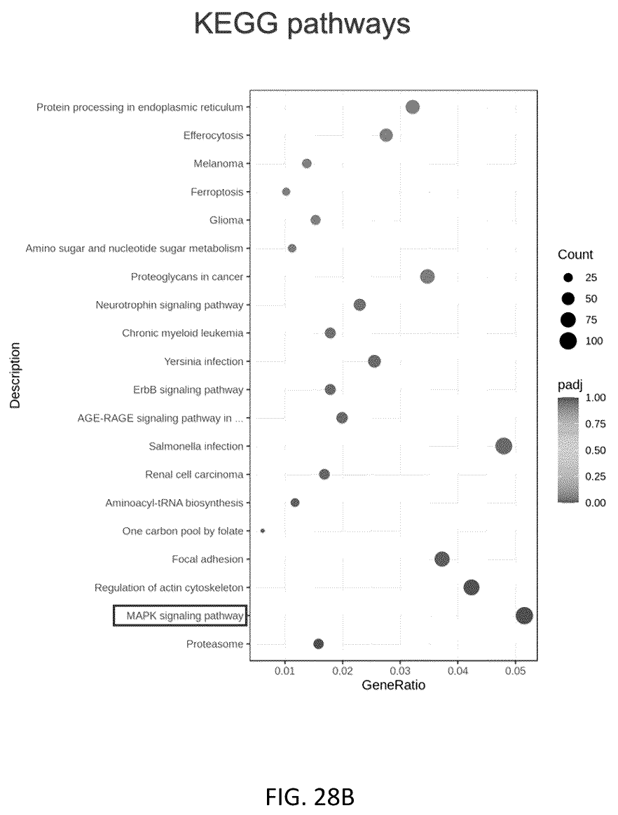

To further investigate the underlying mechanisms and elucidate the signaling pathways influenced by Tff2 during wound healing and repair, transcriptomic analysis using RNA sequencing was performed on E siRNA NC and E siRNA Tff2 cells. The differentially expressed genes (DEGs) identified between the two groups are presented in FIG. 28A. Notably, these DEGs were primarily enriched in the MAPK signaling pathway (FIG. 28B). Specifically, the expression levels of IL1R, MYD88, and P53 were upregulated, while TGF-beta expression was downregulated.

### AAV9-Tff2 Reduced the Bone Loss in Mouse Periodontitis Model

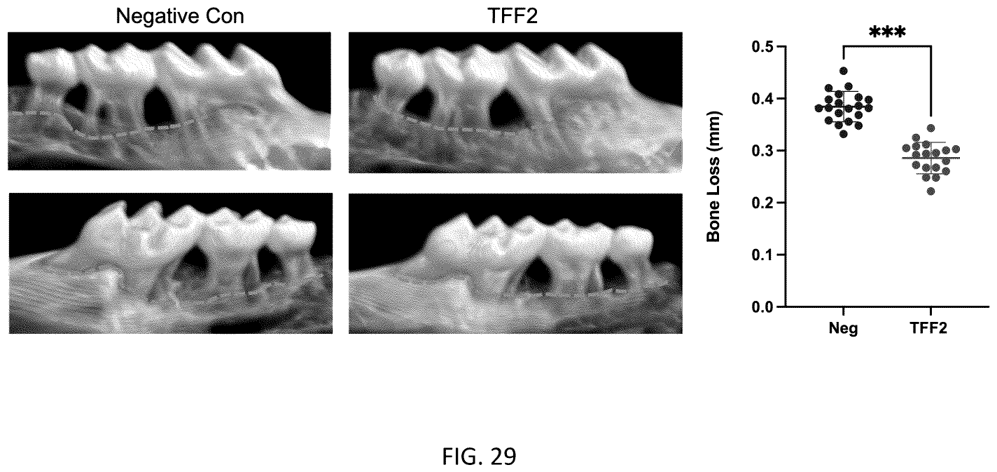

Based on these results, a periodontitis model was established in mice. After ligating the gingiva around the second molar with silk thread for two days, viral solutions carrying Tff2 were injected into the gingiva above the second molar, with an empty vector group serving as the control. The injections were administered three times at five-day intervals, and the animals were sacrificed after two weeks. Micro-CT analysis demonstrated that the Tff2 group showed significantly reduced bone destruction and alveolar bone resorption compared to the control group (FIG. 29). Statistical analysis of alveolar bone resorption confirmed that the reduction in the Tff2 group was statistically significant, providing strong evidence for the therapeutic potential of Tff2 in treating periodontitis (FIG. 29).

We claim:

1. A construct comprising a heterologous promoter operably connected to a nucleic acid sequence encoding an ANRIL RNA or portion thereof or a Trefoil Factor Family 2 protein (Tff2).

2. The construct of claim 1, wherein the nucleic acid sequence encodes the ANRIL RNA and comprises at least one of SEQ ID NOs: 1-32 or a sequence having at least 90% identity to at least one of SEQ ID NOs: 1-32 or portions thereof.

3. The construct of claim 1, wherein the nucleic acid sequence encodes the Tff2 protein and comprises at least one of SEQ ID NO: 89-90, a polynucleotide encoding SEQ ID NO: 91-92 or a sequence having at least 90% identity to at least one of SEQ ID NO: 89-90, 91 or 92.

4. The construct of claim 1, wherein the heterologous promoter comprises a CMV IE promoter or a polymerase III promoter.

5. A viral vector comprising the construct of claim 1.

6. The viral vector of claim 5, wherein the viral vector is an adeno associated virus (AAV).

7. The viral vector of claim 6, wherein the AAV is AAV9.

8. A pharmaceutical composition comprising the construct of claim 1 and a pharmaceutical carrier.

9. A method of using the composition of claim 8 to treat a condition, the method comprising administering a therapeutically effective amount of the composition to a subject in need of treatment for the condition.

10. The method of claim 9, wherein the condition is selected from the group consisting of a wound, inflammation, bone loss, periodontitis, diabetes, metabolic bone disorder and atherosclerosis.

11. The method of claim 10, wherein the condition consists of diabetes-associated periodontal disease.

12. The method of claim 10, wherein the metabolic bone disorder is selected from a group consisting of osteoporosis, osteomalacia, osteoarthritis, and hyperparathyroidism.

13. The method of claim 9, wherein the composition is administered via subcutaneous injection.

14. The method of claim 10, wherein the condition is periodontitis and administration is via gingival injection.

15. The method of claim 9, wherein the subject is a Homo sapiens.

16. The method of claim 9, wherein the nucleic acid sequence encodes the ANRIL RNA or a portion thereof and the portion of ANRIL RNA is capable of binding to miR-99a (aligns with nucleotides 381-420 of lncRNA-APDC of SEQ ID NO: 33).

17. The method of claim 9, further comprising measuring the level of expression of an ANRIL RNA or Tff2 prior to administration of the construct, viral vector or composition, wherein a low level of ANRIL RNA or Tff2 is indicative of a disease in the subject.

18. The method of claim 9, wherein the nucleic acid sequence encodes the ANRIL RNA and comprises at least one of SEQ ID NOs: 1-32 or a sequence having at least 90% identity to at least one of SEQ ID NOs: 1-32 or portions thereof.

19. The method of claim 9, wherein the nucleic acid sequence encodes the Tff2 protein and comprises at least one of SEQ ID NO: 89-90 a polynucleotide encoding SEQ ID NO: 91-92 or a sequence having at least 90% identity to at least one of SEQ ID NO: 89-90, 91 or 92.

20. A method of diagnosing a subject with a condition, the method comprising: measuring the level of expression of an ANRIL RNA or Tff2 in a sample obtained from a subject, comparing the level to a control or reference level; determining whether the level of ANRIL RNA or Tff2 are decrease relative to a control, and administering the construct of claim 1 to the subject, wherein the subject with decreased levels of ANRIL RNA or Tff2 has a condition selected from the group consisting of inflammation, bone loss, periodontitis, diabetes, metabolic bone disorder and atherosclerosis.


SEQUENCE LISTING (98 sequences)

SEQ ID NO: 1 - DNA, 3453 bp, Homo sapiens
  source 1..3453: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgctgcaatttatagcactgatctgtcatcaataccacttgctgtcttggatgtgaagatgatttttcctgcagggattccctctacaaaattaaaaacactgggcatgtggaaataatattcatgctttaaattgtcttttctcttcactacaccaggggtccccaacccctaggccacagactgtggccctagtgtagtgaatagaaaagacaatttaaagcgtgaatattatttcctcatgcccagtgtttttaattttgtactggtctgtggcttgttagaaaccaggctgcacagcagaaggtgggcagcagatattgagaaaccacagaaagagagaagttaaataattttcctgtcaaagaccacatcaatgatgaagccagaatttgagctcatgtacttaaccactggactacctgcctgccctgtcgaggaacagctaagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagtctctccaatgaacgccttcactgatatccaaagcatgaaggacacaccagggaaaaacatagacctaacacaggacaaatggaattattagaaacattttctagcagaagaacactattctgttgccatttgaatctttgcttctttctaggtttgacaatgagcctatcatataagcccaaatgtaaacagaaagaggttgaatcagtcacgataagcccaattatgctgtggtaacaaacaacctcaaaatctcattggcttaaaatatacagaattattcttactcatggcacatatccatctatcatctgcaggggatctgctcactgaagtcacttaggaacttggactgatggaacggccactttttggtcactatatgtattaatctgttttaatcctgctgataaagacccaaaattgggaacaaaaagaagtttaactagacttacagttccgcatggctgaggaggcctcagaatcatggtgggaggcgaaaggcacttcttacatggtggcagcaagagaaaaatgaggaagaagcaaaagcggaaacctctgataaacccatcagatcttatgagacttattccactatcaagagaatagcatgggaaagactggctcccataatttacctccctctgggtccctccctcaacatgtgggaattctgggagaaacaattcaagatatgacacattcataatttaaacagaagcctacgaagaactcataaattaaaagaagataatcttttcacaaggtgatggaggctttttattttgccacaaaaccactggtgacgttgcctgtggccaccttggagaagacactggaggcctgggacatggagactgcttttctgcagaaaccacatcccttggagtaatgagctacacctacctcaattattcagtgcagtacaacactccagacagggtctcactctgtcacccaggctggagtgtattggcatgattacagctcactgcaaccttgaactcccaggctcaaacctgagcagctgggactacagatgcaccaccatgcatggtaccagagatataataatgagaaacagacatgctccctcccctcattgaggttacagcttagtgtggagacacacagatgcctaacgcactatggtatggaaggtgctatggacacagtgctcaaatccatgatctacataggtggagaacttcagtagaggaagtggcaggaatttgggaatgaggagcacagtgattaaactggggccattcatatgagagtttaagaactcagaccagtgacttagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 2 - DNA, 1258 bp, Homo sapiens
  source 1..1258: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacaggttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacatattggcatatgtctttctggtatattttcctgtgttgtcatcattatcatctccatcatcattatatccatcattatcatcatcatcatcatcatcatcatcattatcatcaccatagtgaacatgtaatgcttacctagtgccagatgctgtctaggcattttacatgtgttactggtaactcatgtaatcctcataacaaccttataaggtggttgctattatccccatgttacatatgaagagacagaagcataaagaagttgcaccgctggtaattggctgggatttgaacttaagcagtctaaccttagagtaatgattttaacaactatgctatatacatacaaatttacaaaataaaactgggctcagacaataaaaaaaaaaaaaaaaaaaaaaaaa

SEQ ID NO: 3 - DNA, 2242 bp, Homo sapiens
  source 1..2242: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgctatattgagaaaccacagaaagagagaagttaaataattttcctgtcaaagaccacatcaatgatgaagccagaatttgagctcatgtacttaaccactggactacctgcctgccctgtcgaggaacagctaagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggctttggtatcaggatgattctggcctcataaaatgagttggggagaattccctctttttctattgaatggaatagtttcagaaggaatggtaccagctcctctttgtacctctggtagaattcggctgtgaatccatctggtcctggactttttttggttggtaccagagatataataatgagaaacagacatgctccctcccctcattgaggttacagcttagtgtggagacacacagatgcctaacgcactatggtatggaaggtgctatggacacagtgctcaaatccatgatctacataggtggagaacttcagtagaggaagtggcaggaatttgggaatgaggagcacagtgattaaactggggccattcatatgagagtttaagaactcagaccagtgacttagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 4 - DNA, 1400 bp, Homo sapiens
  source 1..1400: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacaggatgattacggagctaggaatttcctacgaagctgggtgataaaaggacattggacaaaaacacagatagcttcattctataccaggatccatcactgattagctgtattatatttgtctctactgtaatttgaggagaatgatacctgcttctctatcttgttgggttttacagatgatgtattgaaagttcaataaaatctataaacaatagatctctgtggcttgaatggtcctcagatatcttgaccaatgattctcaaatgcttaccatagaccataatttttccccacatgacactgcttcatatttttaagttttttttcaccaaatattttgttttactatctccaagccctgaaactttttttctccctcaaacatgacttcaactttactatcctggtccctttctattgaatgtgttttgtttgtgtctatatttcttatagtgtcttgaactgattgtacttacgatatgacttgactagcataagatagaatagggcagtcatcttctttgatctagactagatgctatccttctattaatgcagttgctagttttgtatttgcttgctttcaatttttttttttcagattttggtaaccactgttacttgactcgtatcaaacttatcagtagctaaaacccccattttccctcttcctgtgcttatttgactagttttgttgcgctttctgaaatgtttctcacttgtttttgaccaaaaaaataaaaataacaaatgcca

SEQ ID NO: 5 - DNA, 1506 bp, Homo sapiens
  source 1..1506: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacaggtggagaacttcagtagaggaagtggcaggaatttgggaatgaggagcacagtgattaaactggggccattcatatgagagtttaagaactcagaccagtgacttagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 6 - DNA, 1401 bp, Homo sapiens
  source 1..1401: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagattggcttctctcacatggcaagaaacattgctgctagcacttcccgagttctacgttctacaacatccaccactgctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 7 - DNA, 1265 bp, Homo sapiens
  source 1..1265: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagattggcttctctcacatggcaagaaacattgctgctagcacttcccgagttctacgttctacaacatccaccactgctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 8 - DNA, 1201 bp, Homo sapiens
  source 1..1201: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 9 - DNA, 1065 bp, Homo sapiens
  source 1..1065: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 10 - DNA, 3204 bp, Homo sapiens
  source 1..3204: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgctgcaatttatagcactgatctgtcatcaataccacttgctgtcttggatgtgaagatgatttttcctgcagggattccctctacaaaattaaaaacactgggcatgtggaaataatattcatgctttaaattgtcttttctcttcactacaccaggggtccccaacccctaggccacagactgtggccctagtgtagtgaatagaaaagacaatttaaagcgtgaatattatttcctcatgcccagtgtttttaattttgtactggtctgtggcttgttagaaaccaggctgcacagcagaaggtgggcagcagatattgagaaaccacagaaagagagaagttaaataattttcctgtcaaagaccacatcaatgatgaagccagaatttgagctcatgtacttaaccactggactacctgcctgccctgtcgaggaacagctaagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagtctctccaatgaacgccttcactgatatccaaagcatgaaggacacaccagggaaaaacatagacctaacacaggacaaatggaattattagaaacattttctagcagaagaacactattctgttgccatttgaatctttgcttctttctaggtttgacaatgagcctatcatataagcccaaatgtaaacagaaagaggttgaatcagtcacgataagcccaattatgctgtggtaacaaacaacctcaaaatctcattggcttaaaatatacagaattattcttactcatggcacatatccatctatcatctgcaggggatctgctcactgaagtcacttaggaacttggactgatggaacggccactttttggtcactatatgtattaatctgttttaatcctgctgataaagacccaaaattgggaacaaaaagaagtttaactagacttacagttccgcatggctgaggaggcctcagaatcatggtgggaggcgaaaggcacttcttacatggtggcagcaagagaaaaatgaggaagaagcaaaagcggaaacctctgataaacccatcagatcttatgagacttattccactatcaagagaatagcatgggaaagactggctcccataatttacctccctctgggtccctccctcaacatgtgggaattctgggagaaacaattcaagatatgacacattcataatttaaacagaagcctacgaagaactcataaattaaaagaagataatcttttcacaaggtgatggaggctttttattttgccacaaaaccactggtgacgttgcctgtggccaccttggagaagacactggaggcctgggacatggagactgcttttctgcagaaaccacatcccttggagtaatgagctacacctacctcaattattcagtgcagtacaacactccagacagggtctcactctgtcacccaggctggagtgtattggcatgattacagctcactgcaaccttgaactcccaggctcaaacctgagcagctgggactacagatgcaccaccatgcatggatgattacggagctaggaatttcctacgaagctgggtgataaaaggacattggacaaaaacacagatagcttcattctataccaggatccatcactgattagctgtattatatttgtctctactgtaatttgaggagaatgatacctgcttctctatcttgttgggttttacagatgatgtattgaaagttcaataaaatctataaacaatagatctctgtggcttgaatggtcctcagatatcttgaccaatgattctcaaatgcttaccatagaccataatttttccccacatgacactgcttcatatttttaagttttttttcaccaaatattttgttttactatctccaagccctgaaactttttttctccctcaaacatgacttcaactttactatcctggtccctttctattgaatgtgttttgtttgtgtctatatttcttatagtgtcttgaactgattgtacttacgatatgacttgactagcataagatagaatagggcagtcatcttctttgatctagactagatgctatccttctattaatgcagttgctagttttgtatttgcttgctttcaatttttttttttcagattttggtaaccactgttacttgactcgtatcaaacttatcagtagctaaaacccccattttccctcttcctgtgcttatttgactagttttgttgcgctttctgaaatgtttctcacttgtttttgaccaaaaaaataaaaataacaaatgcca

SEQ ID NO: 11 - DNA, 1804 bp, Homo sapiens
  source 1..1804: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagatatgacacattcataatttaaacagaagcctacgaagaactcataaattaaaagaagataatcttttcacaaggtgatggaggctttttattttgccacaaaaccactggtgacgttgcctgtggccaccttggagaagacactggagacagggtctcactctgtcacccaggctggagtgtattggcatgattacagctcactgcaaccttgaactcccaggctcaaacctgagcagctgggactacagatgcaccaccatgcatggatgattacggagctaggaatttcctacgaagctgggtgataaaaggacattggacaaaaacacagatagcttcattctataccaggatccatcactgattagctgtattatatttgtctctactgtaatttgaggagaatgatacctgcttctctatcttgttgggttttacagatgatgtattgaaagttcaataaaatctataaacaatagatctctgtggcttgaatggtcctcagatatcttgaccaatgattctcaaatgcttaccatagaccataatttttccccacatgacactgcttcatatttttaagttttttttcaccaaatattttgttttactatctccaagccctgaaactttttttctccctcaaacatgacttcaactttactatcctggtccctttctattgaatgtgttttgtttgtgtctatatttcttatagtgtcttgaactgattgtacttacgatatgacttgactagcataagatagaatagggcagtcatcttctttgatctagactagatgctatccttctattaatgcagttgctagttttgtatttgcttgctttcaatttttttttttcagattttggtaaccactgttacttgactcgtatcaaacttatcagtagctaaaacccccattttccctcttcctgtgcttatttgactagttttgttgcgctttctgaaatgtttctcacttgtttttgaccaaaaaaataaaaataacaaatgcca

SEQ ID NO: 12 - DNA, 1611 bp, Homo sapiens
  source 1..1611: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtgatggaggctttttattttgccacaaaaccactggtgacgttgcctgtggccaccttggagaagacactggaggatgattacggagctaggaatttcctacgaagctgggtgataaaaggacattggacaaaaacacagatagcttcattctataccaggatccatcactgattagctgtattatatttgtctctactgtaatttgaggagaatgatacctgcttctctatcttgttgggttttacagatgatgtattgaaagttcaataaaatctataaacaatagatctctgtggcttgaatggtcctcagatatcttgaccaatgattctcaaatgcttaccatagaccataatttttccccacatgacactgcttcatatttttaagttttttttcaccaaatattttgttttactatctccaagccctgaaactttttttctccctcaaacatgacttcaactttactatcctggtccctttctattgaatgtgttttgtttgtgtctatatttcttatagtgtcttgaactgattgtacttacgatatgacttgactagcataagatagaatagggcagtcatcttctttgatctagactagatgctatccttctattaatgcagttgctagttttgtatttgcttgctttcaatttttttttttcagattttggtaaccactgttacttgactcgtatcaaacttatcagtagctaaaacccccattttccctcttcctgtgcttatttgactagttttgttgcgctttctgaaatgtttctcacttgtttttgaccaaaaaaataaaaataacaaatgcca

SEQ ID NO: 13 - DNA, 1536 bp, Homo sapiens
  source 1..1536: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggatgattacggagctaggaatttcctacgaagctgggtgataaaaggacattggacaaaaacacagatagcttcattctataccaggatccatcactgattagctgtattatatttgtctctactgtaatttgaggagaatgatacctgcttctctatcttgttgggttttacagatgatgtattgaaagttcaataaaatctataaacaatagatctctgtggcttgaatggtcctcagatatcttgaccaatgattctcaaatgcttaccatagaccataatttttccccacatgacactgcttcatatttttaagttttttttcaccaaatattttgttttactatctccaagccctgaaactttttttctccctcaaacatgacttcaactttactatcctggtccctttctattgaatgtgttttgtttgtgtctatatttcttatagtgtcttgaactgattgtacttacgatatgacttgactagcataagatagaatagggcagtcatcttctttgatctagactagatgctatccttctattaatgcagttgctagttttgtatttgcttgctttcaatttttttttttcagattttggtaaccactgttacttgactcgtatcaaacttatcagtagctaaaacccccattttccctcttcctgtgcttatttgactagttttgttgcgctttctgaaatgtttctcacttgtttttgaccaaaaaaataaaaataacaaatgcca

SEQ ID NO: 14 - DNA, 1535 bp, Homo sapiens
  source 1..1535: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 15 - DNA, 1642 bp, Homo sapiens
  source 1..1642: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtggagaacttcagtagaggaagtggcaggaatttgggaatgaggagcacagtgattaaactggggccattcatatgagagtttaagaactcagaccagtgacttagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 16 - DNA, 2237 bp, Homo sapiens
  source 1..2237: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgcttgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtgatggaggctttttattttgccacaaaaccactggtgacgttgcctgtggccaccttggagaagacactggaggcctgggacatggagactgcttttctgcagaaaccacatcccttggagtaatgagctacacctacctcaattattcagtgcagtacaacactccagacagggtctcactctgtcacccaggctggagtgtattggcatgattacagctcactgcaaccttgaactcccaggctcaaacctgagcagctgggactacagatgcaccaccatgcatggtaccagagatataataatgagaaacagacatgctccctcccctcattgaggttacagcttagtgtggagacacacagatgcctaacgcactatggtatggaaggtgctatggacacagtgctcaaatccatgatctacataggtggagaacttcagtagaggaagtggcaggaatttgggaatgaggagcacagtgattaaactggggccattcatatgagagtttaagaactcagaccagtgacttagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 17 - DNA, 1720 bp, Homo sapiens
  source 1..1720: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgcttgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtgaggaggtattccaatgcaaaagaataataggagcaaaagcacagtggtgagaaattggaggggaactgtgaaaattgccacatagattagaggcaggaaaataaaggacggctaagtttatatagtgaacagtgagccgcatggacacaggtgactgttttctcctttttgaacccctgcttactccagagtcaccacctctcctggctttctgccaatcttcttggccacagtttctcggtcttcttttctggcttctcttccttggcctgaatgctacatgttaagtgatgtcagcttcagttttcttaacctctcttctcttaagttgctatcatttaaatatctttaaatatcatctacatccaagtgacctctaaaccagtaattttacccatgacccatctcctcaactccggattgtatataaaactgtctactcaacatcttcacttttgatgtctaaatctctatgtccaatttagaattcttgattctttgctttccctccctcctggaaatcctgctcttaccagtcttccccatcttaattaatgaacctgacattcacctagtagcttagtcctcaaaactagggattaatctagatttagtcatttttcttactcttgccatataataacatattattctgagtttacctttacccgaccccaaataggtaactgctgtttttgttataagatagtaatatttgcctctgagtgtatgtgaattattttgataaaaataaatattatttttaaaatattaa

SEQ ID NO: 18 - DNA, 1934 bp, Homo sapiens
  source 1..1934: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagatatgacacattcataatttaaacagaagcctacgaagaactcataaattaaaagaagataatcttttcacaaggtgatggaggctttttattttgccacaaaaccactggtgacgttgcctgtggccaccttggagaagacactggaggtaccagagatataataatgagaaacagacatgctccctcccctcattgaggttacagcttagtgtggagacacacagatgcctaacgcactatggtatggaaggtgctatggacacagtgctcaaatccatgatctacataggtggagaacttcagtagaggaagtggcaggaatttgggaatgaggagcacagtgattaaactggggccattcatatgagagtttaagaactcagaccagtgacttagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 19 - DNA, 1811 bp, Homo sapiens
  source 1..1811: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgcttgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacaggtaccagagatataataatgagaaacagacatgctccctcccctcattgaggttacagcttagtgtggagacacacagatgcctaacgcactatggtatggaaggtgctatggacacagtgctcaaatccatgatctacataggtggagaacttcagtagaggaagtggcaggaatttgggaatgaggagcacagtgattaaactggggccattcatatgagagtttaagaactcagaccagtgacttagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 20 - DNA, 1651 bp, Homo sapiens
  source 1..1651: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtaccagagatataataatgagaaacagacatgctccctcccctcattgaggttacagcttagtgtggagacacacagatgcctaacgcactatggtatggaaggtgctatggacacagtgctcaaatccatgatctacataggtggagaacttcagtagaggaagtggcaggaatttgggaatgaggagcacagtgattaaactggggccattcatatgagagtttaagaactcagaccagtgacttagattggcttctctcacatggcaagaaacattgctgctagcacttcccgagttctacgttctacaacatccaccactgctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 21 - DNA, 1853 bp, Homo sapiens
  source 1..1853: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgctatattgagaaaccacagaaagagagaagttaaataattttcctgtcaaagaccacatcaatgatgaagccagaatttgagctcatgtacttaaccactggactacctgcctgccctgtcgaggaacagctaagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtgaggaggtattccaatgcaaaagaataataggagcaaaagcacagtggtgagaaattggaggggaactgtgaaaattgccacatagattagaggcaggaaaataaaggacggctaagtttatatagtgaacagtgagccgcatggacacaggtgactgttttctcctttttgaacccctgcttactccagagtcaccacctctcctggctttctgccaatcttcttggccacagtttctcggtcttcttttctggcttctcttccttggcctgaatgctacatgttaagtgatgtcagcttcagttttcttaacctctcttctcttaagttgctatcatttaaatatctttaaatatcatctacatccaagtgacctctaaaccagtaattttacccatgacccatctcctcaactccggattgtatataaaactgtctactcaacatcttcacttttgatgtctaaatctctatgtccaatttagaattcttgattctttgctttccctccctcctggaaatcctgctcttaccagtcttccccatcttaattaatgaacctgacattcacctagtagcttagtcctcaaaactagggattaatctagatttagtcatttttcttactcttgccatataataacatattattctgagtttacctttacccgaccccaaataggtaactgctgtttttgttataagatagtaatatttgcctctgagtgtatgtgaattattttgataaaaataaatattatttttaaaatattaa

SEQ ID NO: 22 - DNA, 2166 bp, Homo sapiens
  source 1..2166: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgctgcaatttatagcactgatctgtcatcaataccacttgctgtcttggatgtgaagatgatttttcctgcagggattccctctacaaaattaaaaacactgggcatgtggaaataatattcatgctttaaattgtcttttctcttcactacaccaggggtccccaacccctaggccacagactgtggccctagtgtagtgaatagaaaagacaatttaaagcgtgaatattatttcctcatgcccagtgtttttaattttgtactggtctgtggcttgttagaaaccaggctgcacagcagaaggtgggcagcagatattgagaaaccacagaaagagagaagttaaataattttcctgtcaaagaccacatcaatgatgaagccagaatttgagctcatgtacttaaccactggactacctgcctgccctgtcgaggaacagctaagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtgaggaggtattccaatgcaaaagaataataggagcaaaagcacagtggtgagaaattggaggggaactgtgaaaattgccacatagattagaggcaggaaaataaaggacggctaagtttatatagtgaacagtgagccgcatggacacaggtgactgttttctcctttttgaacccctgcttactccagagtcaccacctctcctggctttctgccaatcttcttggccacagtttctcggtcttcttttctggcttctcttccttggcctgaatgctacatgttaagtgatgtcagcttcagttttcttaacctctcttctcttaagttgctatcatttaaatatctttaaatatcatctacatccaagtgacctctaaaccagtaattttacccatgacccatctcctcaactccggattgtatataaaactgtctactcaacatcttcacttttgatgtctaaatctctatgtccaatttagaattcttgattctttgctttccctccctcctggaaatcctgctcttaccagtcttccccatcttaattaatgaacctgacattcacctagtagcttagtcctcaaaactagggattaatctagatttagtcatttttcttactcttgccatataataacatattattctgagtttacctttacccgaccccaaataggtaactgctgtttttgttataagatagtaatatttgcctctgagtgtatgtgaattattttgataaaaataaatattatttttaaaatattaa

SEQ ID NO: 23 - DNA, 1558 bp, Homo sapiens
  source 1..1558: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtgaggaggtattccaatgcaaaagaataataggagcaaaagcacagtggtgagaaattggaggggaactgtgaaaattgccacatagattagaggcaggaaaataaaggacggctaagtttatatagtgaacagtgagccgcatggacacaggtgactgttttctcctttttgaacccctgcttactccagagtcaccacctctcctggctttctgccaatcttcttggccacagtttctcggtcttcttttctggcttctcttccttggcctgaatgctacatgttaagtgatgtcagcttcagttttcttaacctctcttctcttaagttgctatcatttaaatatctttaaatatcatctacatccaagtgacctctaaaccagtaattttacccatgacccatctcctcaactccggattgtatataaaactgtctactcaacatcttcacttttgatgtctaaatctctatgtccaatttagaattcttgattctttgctttccctccctcctggaaatcctgctcttaccagtcttccccatcttaattaatgaacctgacattcacctagtagcttagtcctcaaaactagggattaatctagatttagtcatttttcttactcttgccatataataacatattattctgagtttacctttacccgaccccaaataggtaactgctgtttttgttataagatagtaatatttgcctctgagtgtatgtgaattattttgataaaaataaatattatttttaaaatattaa

SEQ ID NO: 24 - DNA, 1820 bp, Homo sapiens
  source 1..1820: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagatgtagtgtcactctgtcgcccaggctggagtgcagtggcgtgatctcggctcactgcaacctctgctacccaggttcaagcgattctcctgcctcagcctcccaaggagctgggattgcaggcacctgccaccacgcccagctaatttttgtagttttggtagagatggtgtttcaccattttggccaggctggtcttaaactcctgaccttgtgatccacctgccttcgcctcccaaagtgctggtattacaggcatgagccactgtgcccagcctaaatgttttctttaaaagcaaggtaagtatgcctagatgcactgcctcttagtaatttttgaggatggattattcattaaattgcttccctttctcacagctggtgaattagaacgatagtgagcttacaagagctttaacgaggaaaagatggtcattgtttaacagagaaggatggtttgggtgagcatttttgtgtttttggaggggaactttgagggcaatgagtgttgcacctgtgtccactgaggatctctgggcggggtaaaaggaaggcagggatgagagtgaggaagggagacaggagggtcccaaatacgaactttgacttaacgaggatgattactagaagggaaagattgtggattcattgatgagatgttgaaccatgaaaactctaattgtagaactcacttcagactgaattgacttggctttcagtagcagaagccctttgtctacttctctagtattgaagtgaatgcaaatttctcaatgtaagaaaagtagagtggtgaaataagagtgtgggttccagggtcagacaactgggtgcataaaaagggtaaggatgctacaactggtaagccctgtgagaattatttaatccttccatgtctcattccgctcatctggaaaatgatgatcatactaacttatgagatgtgtgtacagactagatataatattaaaacatttctgatacctatgcattatattggatcaatatatatttgcaataaatgatcaactttttattaa

SEQ ID NO: 25 - DNA, 1721 bp, Homo sapiens
  source 1..1721: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtgatggaggctttttattttgccacaaaaccactggtgacgttgcctgtggccaccttggagaagacactggagacagggtctcactctgtcacccaggctggagtgtattggcatgattacagctcactgcaaccttgaactcccaggctcaaacctgagcagctgggactacagatgcaccaccatgcatggtaccagagatataataatgagaaacagacatgctccctcccctcattgaggttacagcttagtgtggagacacacagatgcctaacgcactatggtatggaaggtgctatggacacagtgctcaaatccatgatctacataggtggagaacttcagtagaggaagtggcaggaatttgggaatgaggagcacagtgattaaactggggccattcatatgagagtttaagaactcagaccagtgacttagattggcttctctcacatggcaagaaacattgctgctagcacttcccgagttctacgttctacaacatccaccactgtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 26 - DNA, 1435 bp, Homo sapiens
  source 1..1435: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagtagagacggggtttcaccatgttggccagactggtcttgaactctcgacctcgtgattcgcccgcctcggcctcccaaagtgctgggattacaggtgtgagacaccacacccggcggatagagagaattttgacaggtgaggaggtattccaatgcaaaagaataataggagcaaaagcacagtggtgagaaattggaggggaactgtgaaaattgccacatagattagaggcaggaaaataaaggacggctaagtttatatagtgaacagtgagccgcatggacacaggtgactgttttctcctttttgaacccctgcttactccagagtcaccacctctcctggctttctgccaatcttcttggccacagtttctcggtcttcttttctggcttctcttccttggcctgaatgctacatgttaagtgatgtcagcttcagttttcttaacctctcttctcttaagttgctatcatttaaatatctttaaatatcatctacatccaagtgacctctaaaccagtaattttacccatgacccatctcctcaactccggattgtatataaaactgtctactcaacatcttcacttttgatgtctaaatctctatgtccaatttagaattcttgattctttgctttccctccctcctggaaatcctgctcttaccagtcttccccatcttaattaatgaacctgacattcacctagtagcttagtcctcaaaactagggattaatctagatttagtcatttttcttactcttgccatataataacatattattctgagtttacctttacccgaccccaaataggtaactgctgtttttgttataagatagtaatatttgcctctgagtgtatgtgaattattttgataaaaataaatattatttttaaaatattaa

SEQ ID NO: 27 - DNA, 1561 bp, Homo sapiens
  source 1..1561: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgcttgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 28 - DNA, 1694 bp, Homo sapiens
  source 1..1694: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgctatattgagaaaccacagaaagagagaagttaaataattttcctgtcaaagaccacatcaatgatgaagccagaatttgagctcatgtacttaaccactggactacctgcctgccctgtcgaggaacagctaagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 29 - DNA, 1729 bp, Homo sapiens
  source 1..1729: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtgatggaggctttttattttgccacaaaaccactggtgacgttgcctgtggccaccttggagaagacactggagacagggtctcactctgtcacccaggctggagtgtattggcatgattacagctcactgcaaccttgaactcccaggctcaaacctgagcagctgggactacagatgcaccaccatgcatgattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 30 - DNA, 1563 bp, Homo sapiens
  source 1..1563: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgcttgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagattggcttctctcacatggcaagaaacattgctgctagcacttcccgagttctacgttctacaacatccaccactgctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 31 - DNA, 1470 bp, Homo sapiens
  source 1..1470: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgcttgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacaggtggagaacttcagtagaggaagtggcaggaatttgggaatgaggagcacagtgattaaactggggccattcatatgagagtttaagaactcagaccagtgacttagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 32 - DNA, 2295 bp, Homo sapiens
  source 1..2295: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgctgcaatttatagcactgatctgtcatcaataccacttgctgtcttggatgtgaagatgatttttcctgcagggattccctctacaaaattaaaaacactgggcatgtggaaataatattcatgctttaaattgtcttttctcttcactacaccaggggtccccaacccctaggccacagactgtggccctagtgtagtgaatagaaaagacaatttaaagcgtgaatattatttcctcatgcccagtgtttttaattttgtactggtctgtggcttgttagaaaccaggctgcacagcagaaggtgggcagcagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagatgtagtgtcactctgtcgcccaggctggagtgcagtggcgtgatctcggctcactgcaacctctgctacccaggttcaagcgattctcctgcctcagcctcccaaggagctgggattgcaggcacctgccaccacgcccagctaatttttgtagttttggtagagatggtgtttcaccattttggccaggctggtcttaaactcctgaccttgtgatccacctgccttcgcctcccaaagtgctggtattacaggcatgagccactgtgcccagcctaaatgttttctttaaaagcaaggtaagtatgcctagatgcactgcctcttagtaatttttgaggatggattattcattaaattgcttccctttctcacagctggtgaattagaacgatagtgagcttacaagagctttaacgaggaaaagatggtcattgtttaacagagaaggatggtttgggtgagcatttttgtgtttttggaggggaactttgagggcaatgagtgttgcacctgtgtccactgaggatctctgggcggggtaaaaggaaggcagggatgagagtgaggaagggagacaggagggtcccaaatacgaactttgacttaacgaggatgattactagaagggaaagattgtggattcattgatgagatgttgaaccatgaaaactctaattgtagaactcacttcagactgaattgacttggctttcagtagcagaagccctttgtctacttctctagtattgaagtgaatgcaaatttctcaatgtaagaaaagtagagtggtgaaataagagtgtgggttccagggtcagacaactgggtgcataaaaagggtaaggatgctacaactggtaagccctgtgagaattatttaatccttccatgtctcattccgctcatctggaaaatgatgatcatactaacttatgagatgtgtgtacagactagatataatattaaaacatttctgatacctatgcattatattggatcaatatatatttgcaataaatgatcaactttttattaa

SEQ ID NO: 33 - DNA, 2069 bp, Mus musculus
  source 1..2069: mol_type = genomic DNA
gcacgccgcggcggaccggtgacaagtcaggtggagaagctcaagtcttgaactctgcttggtttcccaggtacaggctagaccccacccacgatgatcactgaatcaccaggtgctgaggcctctttctgttgtcaagagccagagacaactgaaagagccagagacaattgaaaggtaccataacacattccctacagtaattagggcttcccttaacaaagccaaggctggcacctgggtagatgttctttgttgattacgaagtcttaatgtggactagcttcagaagcttctcagagccaaaccgtcatctctttgcctttctgctacgaagaccacactcccatccaggccacccaccaggttccatttggtggaacccccaccaaggacacctttagctgttgattgctttatagaataaccgggctggatgacagccctgagcagtttgccagtgtggtctagttttctaggtttccctggtgagtcaacaggaatgtatctacttgtctctgggaattgcagttctacatcatcacctaaagtcttccagattttcctgaagagaggcaaatgtgtaaaacacaactgaaagctgactgttgaattaacatcgtggtgaacaagatcttggatgtgagtcatggaaatgaacctgaactgagagttgctttccaaagactatctcaccactgggattcaggctgaacaaaggagttagattcagctatatactcacctcggaagatctggagcaggaaccacggctgagagagaacatgcagggttggtctttctgcatctggtttacctcactcaatatgaaacagggattctctgtatagccctgctgtcctggaactcatgctgcagatgaggctgacccttgaagtgagagagctgcttgcccctgccttcttactgctgagattaaagctaaaaattgaggatgaaagtctaggtgactttctggtggctgtgcagatgagaagtcggacagtggcactgcagtttgctctgattgactctggagcctgtgtttgaagctacagcaatacaacagtctgctccagcgctgagcttgtaggaaggcgaccactccaggccgactcaatggctttagcctccagagttatcgtcgtttggcaggaagtcttaaagaaccccacagcgtgcccttccgcccctggcacatcataagctattttgggatgagtcatcggaattctgtggtttgcatatcctgaaggggaagagaagacactgtatggctaacatttatttgaaatagcaagttggaaagaaaaattcttcgataaaagttcagcattagaacacacaggaaggtaggcttggtgacttctgcatgtgactgtcataagcagatgcttgttgcgcttctcccaagccacgttggaagatgctttccaaatgccttgcgctgagcacatcaccagaatggtttccaagctacaagatggctccaggggacacaggatactcacctggctgagtaaggagggagaagggatctgtaagtgctcccccgtcagctgctacacactctgacatgcagctaataatttatgaacttctaactttggaggctgccttcagatgataagcctcataaaagctaacaagggtatatctttggtaagtcagcgaagcattaaaaggacaaatagccagttgatttattgcttactggaatatgtttgaacatgaaagattaaaaagtgagatttccttttcatttaaaagttatatcagcagaaacacacatacgcactcacaaactcacacactcacatacaacactaaccagactcagcaggttgtattgagatatttatccatatgtgtgtgtgtgtgtgtgtgtgtgtgtgtgtgtatacacacacacatatatatgtatacacatacacatatatataaaacaatgctaattgaagattaaaaggctatgaacttaaaagggaggagggaggggctggaagaaggacaggagggacaaagtgatataactatattttagcttaaaataaaagagatgaactgag

SEQ ID NO: 34 - DNA, 29 bp, synthetic construct
  source 1..29: mol_type = other DNA
aaggtatcctaaatactgtcttcttgcag

SEQ ID NO: 35 - DNA, 30 bp, synthetic construct
  source 1..30: mol_type = other DNA
cgagtcaattttcttcatgtttatcctcca

SEQ ID NO: 36 - DNA, 23 bp, synthetic construct
  source 1..23: mol_type = other DNA
cgtaatacgactcactatagggc

SEQ ID NO: 37 - DNA, 25 bp, synthetic construct
  source 1..25: mol_type = other DNA
tatgaaagcacacttgtgggcgtgt

SEQ ID NO: 38 - DNA, 20 bp, synthetic construct
  source 1..20: mol_type = other DNA
cgcttcggcagcacatatac

SEQ ID NO: 39 - DNA, 20 bp, synthetic construct
  source 1..20: mol_type = other DNA
ttcacgaatttgcgtgtcat

SEQ ID NO: 40 - RNA, 22 nt, synthetic construct
  source 1..22: mol_type = other RNA
aacccgtagatccgatcttgtg

SEQ ID NO: 41 - DNA, 23 bp, synthetic construct
  source 1..23: mol_type = other DNA
tgaagaacgtcaagtccattgtg

SEQ ID NO: 42 - DNA, 19 bp, synthetic construct
  source 1..19: mol_type = other DNA
tcccgctgtacctgacagt

SEQ ID NO: 43 - DNA, 20 bp, synthetic construct
  source 1..20: mol_type = other DNA
cttgcccctgccttcttact

SEQ ID NO: 44 - DNA, 20 bp, synthetic construct
  source 1..20: mol_type = other DNA
gctaaagccattgagtcggc

SEQ ID NO: 45 - DNA, 21 bp, synthetic construct
  source 1..21: mol_type = other DNA
ggactacagatgcaccaccat

SEQ ID NO: 46 - DNA, 20 bp, synthetic construct
  source 1..20: mol_type = other DNA
tgagcactgtgtccatagca

SEQ ID NO: 47 - DNA, 21 bp, synthetic construct
  source 1..21: mol_type = other DNA
tggttactgtcatggcgggta

SEQ ID NO: 48 - DNA, 23 bp, synthetic construct
  source 1..23: mol_type = other DNA
tctcagatcgttgaaccttgcta

SEQ ID NO: 49 - DNA, 19 bp, synthetic construct
  source 1..19: mol_type = other DNA
agccacatcgctcagacac

SEQ ID NO: 50 - DNA, 19 bp, synthetic construct
  source 1..19: mol_type = other DNA
gcccaatacgaccaaatcc

SEQ ID NO: 51 - DNA, 21 bp, synthetic construct
  source 1..21: mol_type = other DNA
aggtcggtgtgaacggatttg

SEQ ID NO: 52 - DNA, 23 bp, synthetic construct
  source 1..23: mol_type = other DNA
tgtagaccatgtagttgaggtca

SEQ ID NO: 53 - DNA, 20 bp, synthetic construct
  source 1..20: mol_type = other DNA
gcctttgaggtttttggtca

SEQ ID NO: 54 - DNA, 20 bp, synthetic construct
  source 1..20: mol_type = other DNA
aacccagacacaagcattcc

SEQ ID NO: 55 - DNA, 23 bp, synthetic construct
  source 1..23: mol_type = other DNA
atctgactttgctccccttaacc

SEQ ID NO: 56 - DNA, 17 bp, synthetic construct
  source 1..17: mol_type = other DNA
gggccctggttgcaaga

SEQ ID NO: 57 - DNA, 21 bp, synthetic construct
  source 1..21: mol_type = other DNA
aacgatctgagatttgtgggc

SEQ ID NO: 58 - DNA, 21 bp, synthetic construct
  source 1..21: mol_type = other DNA
cctgcgtgggatttcttggtt

SEQ ID NO: 59 - DNA, 20 bp, synthetic construct
  source 1..20: mol_type = other DNA
gccggagtctgttcactacc

SEQ ID NO: 60 - DNA, 20 bp, synthetic construct
  source 1..20: mol_type = other DNA
gcgctctgtctctctgacct

SEQ ID NO: 61 - DNA, 21 bp, synthetic construct
  source 1..21: mol_type = other DNA
gagaaaagcgtcaagccaaac

SEQ ID NO: 62 - DNA, 20 bp, synthetic construct
  source 1..20: mol_type = other DNA
ggtgccacgatccagtcatt

SEQ ID NO: 63 - DNA, 27 bp, synthetic construct
  source 1..27: mol_type = other DNA
gaccattagccttgtgtgtactgtatg

SEQ ID NO: 64 - DNA, 22 bp, synthetic construct
  source 1..22: mol_type = other DNA
tggatcgattgtgcttcaagtt

SEQ ID NO: 65 - DNA, 21 bp, synthetic construct
  source 1..21: mol_type = other DNA
actgccacacctccagtcatt

SEQ ID NO: 66 - DNA, 21 bp, synthetic construct
  source 1..21: mol_type = other DNA
ctttgcctcactcaggattgg

SEQ ID NO: 67 - DNA, 20 bp, synthetic construct
  source 1..20: mol_type = other DNA
ctgttgtgggagacacgatg

SEQ ID NO: 68 - DNA, 20 bp, synthetic construct
  source 1..20: mol_type = other DNA
cctcttctcagggctcacag

SEQ ID NO: 69 - DNA, 20 bp, synthetic construct
  source 1..20: mol_type = other DNA
gcagaggcatacttgtaccg

SEQ ID NO: 70 - DNA, 21 bp, synthetic construct
  source 1..21: mol_type = other DNA
tccaggttatgggcagagatt

SEQ ID NO: 71 - DNA, 22 bp, synthetic construct
  source 1..22: mol_type = other DNA
gaagaagactcaccagaagcag

SEQ ID NO: 72 - DNA, 23 bp, synthetic construct
  source 1..23: mol_type = other DNA
tgatgttatgatggtcccacttg

SEQ ID NO: 73 - DNA, 21 bp, synthetic construct
  source 1..21: mol_type = other DNA
gacttttgagttagcggcact

SEQ ID NO: 74 - DNA, 19 bp, synthetic construct
  source 1..19: mol_type = other DNA
ccgccagctcgttttcatc

SEQ ID NO: 75 - DNA, 21 bp, synthetic construct
  source 1..21: mol_type = other DNA
cagcatcgctctgttcctgta

SEQ ID NO: 76 - DNA, 21 bp, synthetic construct
  source 1..21: mol_type = other DNA
ctgcgttttcatggagtctca

SEQ ID NO: 77 - DNA, 23 bp, synthetic construct
  source 1..23: mol_type = other DNA
gctttgttagaacctctactggg

SEQ ID NO: 78 - DNA, 23 bp, synthetic construct
  source 1..23: mol_type = other DNA
ggtcaggcagatataaagggtca

SEQ ID NO: 79 - DNA, 22 bp, synthetic construct
  source 1..22: mol_type = other DNA
caccctcacagattacaccaac

SEQ ID NO: 80 - DNA, 22 bp, synthetic construct
  source 1..22: mol_type = other DNA
ggagaaccatagtgtctgccag

SEQ ID NO: 81 - DNA, 19 bp, synthetic construct
  source 1..19: mol_type = other DNA
tctttttgggtgcagtgct

SEQ ID NO: 82 - DNA, 22 bp, synthetic construct
  source 1..22: mol_type = other DNA
gcaattcctgaatagtccagtt

SEQ ID NO: 83 - DNA, 18 bp, synthetic construct
  source 1..18: mol_type = other DNA
gggcattgcagccattat

SEQ ID NO: 84 - DNA, 20 bp, synthetic construct
  source 1..20: mol_type = other DNA
taggcccaaaaacactcctg

SEQ ID NO: 85 - DNA, 22 bp, synthetic construct
  source 1..22: mol_type = other DNA
gcactggcaagttctactgcaa

SEQ ID NO: 86 - DNA, 23 bp, synthetic construct
  source 1..23: mol_type = other DNA
gtaggtgaagagaacggccttgt

SEQ ID NO: 87 - DNA, 21 bp, synthetic construct
  source 1..21: mol_type = other DNA
gtgccagtttcgatccgtaga

SEQ ID NO: 88 - DNA, 20 bp, synthetic construct
  source 1..20: mol_type = other DNA
ggccagcatcgtgtagatga

SEQ ID NO: 89 - DNA, 571 bp, Mus musculus
  source 1..571: mol_type = genomic DNA
attctgcaggctgcccaggtccagtggagcagacatgcgacctcgaggtgcccccctgctggcagtggtcctggttttgggactgcatgctctggtagagggcgagaaaccttccccctgtcggtgctccaggctgacaccccacaacagaaagaactgtggcttcccgggcatcaccagtgagcagtgctttgatcttggatgctgctttgactctagcgtcgctggggtcccttggtgtttccacccacttccaaaccaagaatcggagcagtgtgtcatggaagtgtcagctcgcaagaattgtgggtacccgggcatcagtcccgaggactgtgccagtcgaaactgctgcttttccaacctgatctttgaagtgccctggtgtttcttcccacagtctgtggaagattgtcactactgagagttgctactgccgagccacccgttccctgggagctgcaagccagaagaaagtttcaaccagacttcatcaatctctggggtttctaaaaccatcttgacccttagcagtggctagacacagcattttccaagtaaagaaaagttg

SEQ ID NO: 90 - DNA, 603 bp, Homo sapiens
  source 1..603: mol_type = genomic DNA
attctgcaagccaccctggggtgcagctgagctagacatgggacggcgagacgcccagctcctggcagcgctcctcgtcctggggctatgtgccctggcggggagtgagaaaccctccccctgccagtgctccaggctgagcccccataacaggacgaactgcggcttccctggaatcaccagtgaccagtgttttgacaatggatgctgtttcgactccagtgtcactggggtcccctggtgtttccaccccctcccaaagcaagagtcggatcagtgcgtcatggaggtctcagaccgaagaaactgtggctacccgggcatcagccccgaggaatgcgcctctcggaagtgctgcttctccaacttcatctttgaagtgccctggtgcttcttcccgaagtctgtggaagactgccattactaagagaggctggttccagaggatgcatctggctcaccgggtgttccgaaaccaaagaagaaacttcgccttatcagcttcatacttcatgaaatcctgggttttcttaaccatcttttcctcattttcaatggtttaacatataatttctttaaataaaacccttaaaatctgctaaa

SEQ ID NO: 91 - protein, 127 aa, Mus musculus
PRGAPLLAVVLVLGLHALVEGEKPSPCRCSRLTPHNRKNCGFPGITSEQCFDLGCCFDSSVAGVPWCFHPLPNQESEQCVMEVSARKNCGYPGISPEDCASRNCCFSNLIFEVPWCFFPQSVEDCHY

SEQ ID NO: 92 - protein, 129 aa, Homo sapiens
MRPRGAPLLAVVLVLGLHALVEGEKPSPCRCSRLTPHNRKNCGFPGITSEQCFDLGCCFDSSVAGVPWCFHPLPNQESEQCVMEVSARKNCGYPGISPEDCASRNCCFSNLIFEVPWCFFPQSVEDCHY

SEQ ID NO: 93 - DNA, 22 bp, synthetic construct
  source 1..22: mol_type = other DNA
gtgttctagcctagatgcccaa

SEQ ID NO: 94 - DNA, 40 bp, synthetic construct
  source 1..40: mol_type = other DNA
tatttcgttagttgtcgatttccacaggaaccacccccaa

SEQ ID NO: 95 - RNA, 21 nt, synthetic construct
  source 1..21: mol_type = other RNA
ggatgctgctttgactctatt

SEQ ID NO: 96 - RNA, 21 nt, synthetic construct
  source 1..21: mol_type = other RNA
tagagtcaaagcagcatcctt

SEQ ID NO: 97 - RNA, 51 nt, synthetic construct
  source 1..51: mol_type = other RNA
tgctgcttttccaacctgatctttgaagtgccctggtgtttcttcccacag

SEQ ID NO: 98 - RNA, 50 nt, synthetic construct
  source 1..50: mol_type = other RNA
aaaagggaggagggaggggctggaagaaggacaggaggaacaaagtgata